SEQUENCE LISTING (101 sequences)

SEQ ID NO: 1 - DNA, 2121 bp, Arthrobacter ureafaciens
atgacagaagttgttgatagagcctcatcaccagcctcaccaggttcaacaacagccgccgccgatggtgccaaggttgccgttgaaccaagagttgatgttgccgccttgggtgaacaattgttgggtagatgggccgacatcagattgcacgccagagatttggccggtagagaagttgttcaaaaggttgaaggtttgacacacacagaacacagatcaagagttttcggtcaattgaagtacttggttgataacaacgccgttcacagagccttcccatcaagattgggtggttcagatgatcacggtggtaacatcgccggtttcgaagaattggttacagccgatccatcattgcaaatcaaggccggtgttcaatggggtttgttcggttcagccgttatgcacttgggtacaagagaacaccacgataagtggttgccaggtatcatgtcattggaaatcccaggttgtttcgccatgacagaaacaggtcacggttcagatgttgcctcaatcgccacaacagccacatacgatgaagaaacacaagagttcgttatcgatacaccattcagagccgcctggaaggattacatcggtaacgccgccaacgatggtttggccgccgttgttttcgcccaattgatcacaagaaaggttaaccacggtgttcacgccttctacgttgatttgcgcgatccagccacaggtgatttcttgccaggtatcggtggtgaagatgatggtatcaagggtggtttgaacggtatcgataacggtagattgcacttcacaaacgttagaatcccaagaacaaacttgttgaacagatacggtgatgttgccgttgatggtacatactcatcaacaatcgaatcaccaggtagaagattcttcacaatgttgggtacattggttcaaggtagagtttcattggatggtgccgccgttgccgcctcaaaggttgccttgcaatcagccatccactacgccgccgaaagaagacaattcaacgccacatcaccaacagaagaagaagttttgttggattaccaaagacaccaaagaagattgttcacaagattggccacaacatacgccgcctcattcgcccacgaacaattgttgcaaaagttcgatgatgttttctcaggtgcccacgatacagatgccgatagacaagatttggaaacattggccgccgccttgaagccattgtcaacatggcacgccttggatacattgcaagaatgtagagaagcctgtggtggtgccggtttcttgatcgaaaacagattcgcctcattgagagccgatttggatgtttacgttacattcgaaggtgataacacagttttgttgcaattggttgccaagagattgttggccgattacgccaaggagttcagaggtgccaacttcggtgttttggccagatacgttgttgatcaagccgccggtgttgccttgcacagaacaggtttgagacaagttgcccaattcgttgccgattcaggttcagttcaaaagtcagccttggccttgagagatgaagaaggtcaaagaacattgttgacagatagagttcaatcaatggttgccgaagttggtgccgccttgaagggtgccggtaagttgccacaacaccaagctgccgccttgttcaaccaacaccaaaacgaattgatcgaagccgcccaagcccacgccgaattgttgcaatgggaagccttcacagaagccttggccaaggttgatgatgccggtacaaaggaagttttgacaagattgagagatttgttcggtttgtcattgatcgaaaagcacttgtcatggtacttgatgaacggtagattgtcaatgcaaagaggtagaacagttggtacatacatcaacagattgttggttaagatcagaccacacgccttggatttggttgatgccttcggttacggtgccgaacacttgagagccgccatcgccacaggtgccgaagccacaagacaagatgaagccagaacatacttcagacaacaaagagcctcaggttcagccccagccgatgaaaagacattgttggccatcaaggccggtaagtcaagagccaagttgtag

SEQ ID NO: 2 - DNA, 1176 bp, Cannabis sativa
atgaaccatttgagggctgaagggccagcttctgtgttggccataggtacggctaacccagaaaacattcttcttcaggatgaatttccagattactattttagagtgaccaaatctgagcacatgactcaactcaaggaaaagtttagaaagatctgtgacaaatcaatgataaggaagcgtaattgtttccttaacgaagaacaccttaagcaaaaccctcgattggttgagcacgaaatgcagacattagatgcacgacaagatatgttggttgttgaagtgccaaagcttggtaaggacgcttgtgccaaggccataaaggaatggggtcaacctaaaagtaaaattacccacttgatattcacgtcagccagcacgacagacatgccaggggcagactaccattgtgcaaaattgttaggtttgtcaccgtcggtcaaacgcgttatgatgtaccaattaggctgctatgggggaggaaccgtcttgaggattgctaaggacattgccgaaaacaacaagggtgcacgagtgcttgctgtctgttgcgacatcatggcttgcctctttagaggtccaagcgagagtgatttggaattactcgtcggccaagcgatcttcggtgacggcgccgctgcggtgattgtaggtgctgaaccagacgaatcggtgggtgaacgtccaattttcgagttggtgagcacgggtcaaacaatattgcctaattctgaagggaccatcggcggacatatcagagaggcagggttaatttttgatttgcacaaggacgttcccatgttgatcagcaacaatatcgagaaatgtttgatcgaggcctttacaccgattggtatctcggattggaactccatcttctggattactcatccaggaggaaaggccatcttggataaggttgaggagaagttgcacttgaaatcagataagttcgtcgatagtcgtcatgtccttagtgaacacggcaacatgtcgtcgtccacggttcttttcgtcatggacgaactcaggaaaaggtccttggaggaagggaagtcaacgacaggagacgggtttgagtggggtgtattgtttggttttggacccggtttgaccgtcgaacgtgttgtggtcagaagcgttcctatcaagtatcatcatcaccatcaccactaa

SEQ ID NO: 3 - DNA, 324 bp, Cannabis sativa
atggccgttaaacacttgatagtgttgaagtttaaggatgaaataacggaagctcaaaaagaagagtttttcaagacttatgtcaatttagttaacattatcccagcgatgaaagatgtgtactggggtaaagacgtgacccagaagaacaaagaagagggttatacacatattgtggaggttacgttcgagtcagtggaaacgatccaggattacattatccatccagcccacgtcggattcggggatgtttacagatcattctgggaaaaacttttgatcttcgattatacgccgcggaaacatcatcaccatcaccactaa

SEQ ID NO: 4 - DNA, 1158 bp, Cannabis sativa
atgaaccatttgagggctgaagggccagcttctgtgttggccataggtacggctaacccagaaaacattcttcttcaggatgaatttccagattactattttagagtgaccaaatctgagcacatgactcaactcaaggaaaagtttagaaagatctgtgacaaatcaatgataaggaagcgtaattgtttccttaacgaagaacaccttaagcaaaaccctcgattggttgagcacgaaatgcagacattagatgcacgacaagatatgttggttgttgaagtgccaaagcttggtaaggacgcttgtgccaaggccataaaggaatggggtcaacctaaaagtaaaattacccacttgatattcacgtcagccagcacgacagacatgccaggggcagactaccattgtgcaaaattgttaggtttgtcaccgtcggtcaaacgcgttatgatgtaccaattaggctgctatgggggaggaaccgtcttgaggattgctaaggacattgccgaaaacaacaagggtgcacgagtgcttgctgtctgttgcgacatcatggcttgcctctttagaggtccaagcgagagtgatttggaattactcgtcggccaagcgatcttcggtgacggcgccgctgcggtgattgtaggtgctgaaccagacgaatcggtgggtgaacgtccaattttcgagttggtgagcacgggtcaaacaatattgcctaattctgaagggaccatcggcggacatatcagagaggcagggttaatttttgatttgcacaaggacgttcccatgttgatcagcaacaatatcgagaaatgtttgatcgaggcctttacaccgattggtatctcggattggaactccatcttctggattactcatccaggaggaaaggccatcttggataaggttgaggagaagttgcacttgaaatcagataagttcgtcgatagtcgtcatgtccttagtgaacacggcaacatgtcgtcgtccacggttcttttcgtcatggacgaactcaggaaaaggtccttggaggaagggaagtcaacgacaggagacgggtttgagtggggtgtattgtttggttttggacccggtttgaccgtcgaacgtgttgtggtcagaagcgttcctatcaagtattaa

SEQ ID NO: 5 - DNA, 306 bp, Cannabis sativa
atggccgttaaacacttgatagtgttgaagtttaaggatgaaataacggaagctcaaaaagaagagtttttcaagacttatgtcaatttagttaacattatcccagcgatgaaagatgtgtactggggtaaagacgtgacccagaagaacaaagaagagggttatacacatattgtggaggttacgttcgagtcagtggaaacgatccaggattacattatccatccagcccacgtcggattcggggatgtttacagatcattctgggaaaaacttttgatcttcgattatacgccgcggaaataa

SEQ ID NO: 6 - DNA, 1188 bp, Cannabis sativa
atgggtttgtcctctgtgtgtacattcagcttccaaacgaactatcatacgttgttaaacccacacaacaataaccccaagacttccttgctatgctaccgacacccaaaaacccccataaagtattcttacaacaatttcccctcaaagcactgttcgacaaagagcttccacttgcagaataagtgctccgaaagtttgtctatcgccaagaactcaatcagggctgcaacaaccaaccaaaccgaacctccagaatcggataaccactccgttgctacgaaaatcttgaactttggtaaggcatgttggaagttgcaacgaccgtacacaattatcgccttcacctcatgcgcctgtggtcttttcggtaaggaattactacataacacaaacctaatcagctggtcattaatgtttaaagcgttcttcttcctcgtagccatcttgtgtattgctagttttaccactactattaatcaaatctacgacttgcacattgatagaatcaacaaaccagatctacctttggcatccggagaaattagtgttaatacagcatggatcatgagcattatcgtggcactctttgggttgattatcacgataaagatgaagggcggtccattgtacatattcgggtactgtttcggaatctttggagggattgtttacagcgttccaccatttcgttggaaacaaaatccgagtacagcattcctattgaacttcttggcacacatcataacaaatttcacattctactacgcttcccgggccgccttgggtctcccgtttgagttgagacctagtttcacatttcttttggctttcatgaagtctatgggttcggcattggccctaatcaaggatgcctctgatgtcgaaggtgatacaaagttcggaatttcaacgctcgcatctaagtacgggagccgaaacttgacccttttctgctctgggattgtgttgttgtcgtatgtagccgcaatcttggcaggaattatatggccccaagcgtttaattccaatgttatgcttctttctcatgctatattggccttctggttgattctccaaactagggattttgccctcacaaattacgatccagaagccggaagacgtttttatgaatttatgtggaagttgtactacgctgaatatttggtttacgtgtttatctga

SEQ ID NO: 7 - DNA, 966 bp, Cannabis sativa
atggctgcaacaaccaaccaaaccgaacctccagaatcggataaccactccgttgctacgaaaatcttgaactttggtaaggcatgttggaagttgcaacgaccgtacacaattatcgccttcacctcatgcgcctgtggtcttttcggtaaggaattactacataacacaaacctaatcagctggtcattaatgtttaaagcgttcttcttcctcgtagccatcttgtgtattgctagttttaccactactattaatcaaatctacgacttgcacattgatagaatcaacaaaccagatctacctttggcatccggagaaattagtgttaatacagcatggatcatgagcattatcgtggcactctttgggttgattatcacgataaagatgaagggcggtccattgtacatattcgggtactgtttcggaatctttggagggattgtttacagcgttccaccatttcgttggaaacaaaatccgagtacagcattcctattgaacttcttggcacacatcataacaaatttcacattctactacgcttcccgggccgccttgggtctcccgtttgagttgagacctagtttcacatttcttttggctttcatgaagtctatgggttcggcattggccctaatcaaggatgcctctgatgtcgaaggtgatacaaagttcggaatttcaacgctcgcatctaagtacgggagccgaaacttgacccttttctgctctgggattgtgttgttgtcgtatgtagccgcaatcttggcaggaattatatggccccaagcgtttaattccaatgttatgcttctttctcatgctatattggccttctggttgattctccaaactagggattttgccctcacaaattacgatccagaagccggaagacgtttttatgaatttatgtggaagttgtactacgctgaatatttggtttacgtgtttatctga

SEQ ID NO: 8 - DNA, 1554 bp, Cannabis sativa
atgaaccctagagaaaacttcctcaagtgtttttcccaatatatcccaaacaatgctacaaatcttaaattggtctatacccagaacaatcccttatacatgagtgtccttaactcaacaatccataatctccgtttcacatcggacacgactccaaaaccgttggtaatagtgactccgtcccacgtcagtcacattcagggcactatcctctgttccaagaaggtcggtttgcaaattagaacaagatcgggcgggcacgatagcgaggggatgtcgtacatctctcaagttccattcgttatcgttgacttgcgaaatatgcgctccattaagattgatgttcatagccaaaccgcgtgggttgaagcaggcgctactcttggggaggtctactattgggtgaacgaaaaaaatgaaaacttgtcactcgctgcaggctactgcccaacggtgtgtgctggcggacattttggtggtggggggtatggtcccctcatgcgaaattacgggttagccgcagataatataatagacgctcacctcgtgaacgtccacggtaaagttctagatcggaagtccatgggtgaagatttgttctgggctcttcggggaggtggtgcagagtcctttggaatcatcgttgcgtggaaaattaggttggtggctgtccccaagtctacgatgttttctgttaaaaagatcatggagatccatgagctagttaagttagttaataagtggcaaaacattgcctataagtatgacaaggatttgcttctaatgacgcacttcatcactagaaacatcaccgacaaccaaggcaaaaacaagaccgcaatacacacttacttcagctcagtatttttaggaggggtggattcattggttgacttgatgaataaatcgttcccagaattgggtatcaagaaaaccgattgtcgacagttgtcatggattgatactattatattttacagtggagtggttaactacgacacagacaactttaacaaggagattttgttggacagatctgccgggcagaacggggcctttaaaataaagcttgattatgttaaaaagcccatcccagagagcgtgttcgtccaaatcttggaaaagttgtacgaagaggatatcggcgcaggcatgtacgctttgtacccctacggtggtattatggatgaaatctcggagtccgcaattccttttccccatagagccggtattttgtacgagttgtggtacatttgttcgtgggagaaacaagaggacaacgaaaagcatttgaactggatacggaacatttataacttcatgacaccatacgttagtaagaacccgaggttggcttacttaaactatcgagacctcgacattgggatcaacgatccgaaaaacccaaacaactacacacaagcccgcatctggggagaaaagtactttggaaagaacttcgatagattggtcaaggtgaagacacttgtggacccaaacaacttcttcagaaacgagcagtctattccacctttgcctaggcatagacactga

SEQ ID NO: 9 - DNA, 1635 bp, Cannabis sativa
atgaagtgctcgacattctctttctggttcgtttgtaagattatcttctttttttttagtttcaacatccagacctcaattgctaaccctagagaaaacttcctcaagtgtttttcccaatatatcccaaacaatgctacaaatcttaaattggtctatacccagaacaatcccttatacatgagtgtccttaactcaacaatccataatctccgtttcacatcggacacgactccaaaaccgttggtaatagtgactccgtcccacgtcagtcacattcagggcactatcctctgttccaagaaggtcggtttgcaaattagaacaagatcgggcgggcacgatagcgaggggatgtcgtacatctctcaagttccattcgttatcgttgacttgcgaaatatgcgctccattaagattgatgttcatagccaaaccgcgtgggttgaagcaggcgctactcttggggaggtctactattgggtgaacgaaaaaaatgaaaacttgtcactcgctgcaggctactgcccaacggtgtgtgctggcggacattttggtggtggggggtatggtcccctcatgcgaaattacgggttagccgcagataatataatagacgctcacctcgtgaacgtccacggtaaagttctagatcggaagtccatgggtgaagatttgttctgggctcttcggggaggtggtgcagagtcctttggaatcatcgttgcgtggaaaattaggttggtggctgtccccaagtctacgatgttttctgttaaaaagatcatggagatccatgagctagttaagttagttaataagtggcaaaacattgcctataagtatgacaaggatttgcttctaatgacgcacttcatcactagaaacatcaccgacaaccaaggcaaaaacaagaccgcaatacacacttacttcagctcagtatttttaggaggggtggattcattggttgacttgatgaataaatcgttcccagaattgggtatcaagaaaaccgattgtcgacagttgtcatggattgatactattatattttacagtggagtggttaactacgacacagacaactttaacaaggagattttgttggacagatctgccgggcagaacggggcctttaaaataaagcttgattatgttaaaaagcccatcccagagagcgtgttcgtccaaatcttggaaaagttgtacgaagaggatatcggcgcaggcatgtacgctttgtacccctacggtggtattatggatgaaatctcggagtccgcaattccttttccccatagagccggtattttgtacgagttgtggtacatttgttcgtgggagaaacaagaggacaacgaaaagcatttgaactggatacggaacatttataacttcatgacaccatacgttagtaagaacccgaggttggcttacttaaactatcgagacctcgacattgggatcaacgatccgaaaaacccaaacaactacacacaagcccgcatctggggagaaaagtactttggaaagaacttcgatagattggtcaaggtgaagacacttgtggacccaaacaacttcttcagaaacgagcagtctattccacctttgcctaggcatagacactga

SEQ ID NO: 10 - DNA, 2163 bp, Cannabis sativa
atgggcaagaactacaagagtttagactcggttgtggctagtgactttatcgctctcggcatcacgtcggaagtggctgagaccttgcacggtaggttggccgaaattgtgtgcaattatggagctgctacaccacaaacctggattaacatcgcaaatcatattctatcccctgacttgccttttagtttgcaccagatgttgttctatggttgctataaggatttcggcccagcaccaccagcgtggattcccgacccagaaaaagtcaaatcaacgaacttaggtgcgttgttggagaaaagagggaaggaattcttgggagtgaagtataaagatcctattagctcattctctcactttcaagaattttccgttagaaacccagaggtatattggcgaacagtgttgatggatgagatgaagatttctttctcaaaggacccagaatgcattctaagacgagacgatataaacaacccgggcggatctgaatggttgcccggcggatatctaaactcggcaaagaactgcttaaatgtgaactcaaacaagaagttgaacgatactatgatagtttggcgggacgaaggaaacgacgacttgcccctaaataaattaacgttggaccaattgagaaagcgagtttggcttgttgggtatgcgctagaagaaatgggattggaaaaggggtgtgctattgcaattgatatgccaatgcatgttgacgcggtggtcatttacttggcaatcgtgctagctggatacgtggtcgttagtatagctgactctttttcagccccagaaatcagcacgaggttgcgcttgtcaaaggctaaggccattttcacgcaagaccacatcatcagaggcaagaagcgtataccgttgtactctcgtgttgtcgaggccaaatcccctatggccatcgtcattccatgctcaggtagtaatattggtgctgagctccgagatggagacatatcttgggattatttcttggaaagagcaaaagagttcaaaaactgcgagttcactgcgagggagcaaccggtcgatgcgtacactaacatattatttagttcgggcaccacgggcgagccaaaagcaattccgtggacgcaggctactccactaaaggctgcagctgatggctggtcgcacttggacattagaaagggtgatgtgattgtgtggccaacgaacttgggttggatgatggggccatggttagtctacgcgtctttgttgaatggtgcctcgatcgcgctatacaatggtagtccattggtctctgggttcgccaaattcgtccaagacgccaaggttacgatgttgggggtcgttccgtccattgtgagatcgtggaagagtacaaactgtgtttcaggatatgattggtccaccatcagatgtttctcaagctcaggcgaagcgtcgaatgtcgatgagtacttgtggctcatggggcgtgctaactataaaccagttattgagatgtgtggtggcaccgaaatcggtggtgccttcagtgcaggttctttcttacaggcgcagagtttgagttctttctcatcccaatgcatggggtgcacattgtatatcttggacaagaatgggtatccgatgccaaagaacaaaccgggcataggagagttggccctcggtcccgtgatgttcggggctagcaagactttgttgaacgggaaccaccatgatgtgtacttcaagggaatgccaacattgaatggggaggtcttgagacgtcacggggatatcttcgaattaacctccaacggttattaccacgctcatggtagagcagatgacacaatgaacattggtggcatcaagatatcatcaatagagatagaaagggtatgtaacgaggttgacgatagagtttttgaaacaactgcaattggtgttccgcccttgggtggcggtccagaacagcttgttattttcttcgttttgaaagactcaaacgatactacaatcgatcttaatcaattgagattatcgttcaacctaggcttgcagaagaagttaaatcccttgttcaaggtaacaagggttgttccgttgtcgtcattgccacgcactgctactaacaagataatgagaagagttttgcgacaacaatttagtcatttcgaatga

SEQ ID NO: 11 - DNA, 2223 bp, Candida viswanathii
atgaccactttgccttcgatcgcagacaccgacaacatctatgcctccgacgataaggagtacattttcgacaaccccaacgacttgtccattgagactctcgtcaaccacatccttccatttcctcaggaagttgctggtgaatctgtcaaggttcctggcactgctgtccagggtttctctgaattctacagaaatgctgctactcccaacgggatcaagttgagtttgatcaaggggttggacacttatcatcatatatttgagagctctgctgagcgttacgctgacgacccgtgccttgcgttccacgagtacgactacgagaactcgcagcatttagagcgatacgcaaccatctcctacaaggaagtgcgccaaagaaaggatgattttgccgctggtttgttctttctcttgaaggctaacccttacaagaacgattccttggaggcacatcaaaaaatcgtcaaccatgaagccaactacaagctgtacgacagcgacaacatgtccttcattgtcacgttctacgccgcaaatagagtcgagtgggtcttgtctgacttggcgtgctcctccaattccattacatcgacggcattgtacgacacgttgggcccagatacgtccaagtatattttggagactaccgaatctcctgtcattattagctccaaggaccatattcgcgacttgattgacttgaagaaggcaaaccccaaggaacttgctgctcttatcttgattatttcgatggacccgttgaagaaatctgaccagaacttggttcatttggcagaagcaaacaatatcaagttgtatgatttctcccaagtcgaaagaactggagctattttcccccatcaaaccaatgccccaaacagcgaaactgtcttcacaatcaccttcacttcaggaactactggtgccaatccaaaaggtgttgtccttcctcaacgatgtgctgcctcgggtatgttggcgtatagtgttatgatgcctcaccacaggggcacgagggagtttgcgttcttgccattagcacacatttttgagagacagatggttgcttcgatgtttatgtttggtggctcgtccgcaatgccacgattgggcggtacgccattgaccttggtagaagacttgaagttgtggaaacctacgttcatggccaatgttccacgtgttttcaccaagattgaagcgggcatcaaagcttcgacgatcgactccacttccagcctcacgagatcattgtatgaacgtgctatcgaagcaaagcgtgtcaagcagaacaagaacgacgacagtggagaccactttatctatgacaagttgttgattcaaagattgagaagcgctatcgggtatgactgtttggaattctgtgtcactggtagtgctccaattgctcctgaaactatcaaattcttgaaagctagtttgggaattggatttggtcaaggatatggtagcagtgaatcgtttgctggaatgttgtttgctttgcctttcaagaactccagtgtcggaacctgtggcgtcatctcgcccactatggaggccagattaagagagttaccagacatgggttacatgttgaacgataagaatggaccacgaggagagttgcaactccgtggttctcaattattcaccaggtactacaagaatccagaagaaactgcaaagtccatcgatgaagacggttggttcagtaccggtgacgttgctgagattggcacagatggctatttcagaattattgacagggtgaagaacttctacaaattatcccagggtgagtatgtttcgccagagaagattgagagtttgtacttgtcgttgaactcgagtattctgcagctttttatccatggggactctaccaagtcatttctcgtgggtgtggtgggtttacagcctgatgttgccagcaagtatgttgatctttcttccggtcccaatgtggtccaagtgttaaaccaacctgagtttagaaagcaattattgttagacctaaacctgaaggtcaatggcaaattgcaagggtttgaaaagttgcacaacattttcatcgacattgaaccattgacactcgagagaaatgttgttaccccaacaatgaagctcaagagacattttgctgccaagtttttcaagccccagatcgaagctatgtatgcagaaggctccattgtcaaagattacaagttgtga

SEQ ID NO: 12 - DNA, 2214 bp, Candida viswanathii
atgaccactttgccttcgatcgcagacaccgacaacatctatgcctccgacgataaggagtacattttcgacaaccccaacgacttgtccattgagactctcgtcaaccacatccttccatttcctcaggaagttgctggtgaatctgtcaaggttcctggcactgctgtccagggtttctctgaattctacagaaatgctgctactcccaacgggatcaagttgagtttgatcaaggggttggacacttatcatcatatatttgagagctctgctgagcgttacgctgacgacccgtgccttgcgttccacgagtacgactacgagaactcgcagcatttagagcgatacgcaaccatctcctacaaggaagtgcgccaaagaaaggatgattttgccgctggtttgttctttctcttgaaggctaacccttacaagaacgattccttggaggcacatcaaaaaatcgtcaaccatgaagccaactacaagctgtacgacagcgacaacatgtccttcattgtcacgttctacgccgcaaatagagtcgagtgggtcttgtctgacttggcgtgctcctccaattccattacatcgacggcattgtacgacacgttgggcccagatacgtccaagtatattttggagactaccgaatctcctgtcattattagctccaaggaccatattcgcgacttgattgacttgaagaaggcaaaccccaaggaacttgctgctcttatcttgattatttcgatggacccgttgaagaaatctgaccagaacttggttcatttggcagaagcaaacaatatcaagttgtatgatttctcccaagtcgaaagaactggagctattttcccccatcaaaccaatgccccaaacagcgaaactgtcttcacaatcaccttcacttcaggaactactggtgccaatccaaaaggtgttgtccttcctcaacgatgtgctgcctcgggtatgttggcgtatagtgttatgatgcctcaccacaggggcacgagggagtttgcgttcttgccattagcacacatttttgagagacagatggttgcttcgatgtttatgtttggtggctcgtccgcaatgccacgattgggcggtacgccattgaccttggtagaagacttgaagttgtggaaacctacgttcatggccaatgttccacgtgttttcaccaagattgaagcgggcatcaaagcttcgacgatcgactccacttccagcctcacgagatcattgtatgaacgtgctatcgaagcaaagcgtgtcaagcagaacaagaacgacgacagtggagaccactttatctatgacaagttgttgattcaaagattgagaagcgctatcgggtatgactgtttggaattctgtgtcactggtagtgctccaattgctcctgaaactatcaaattcttgaaagctagtttgggaattggatttggtcaaggatatggtagcagtgaatcgtttgctggaatgttgtttgctttgcctttcaagaactccagtgtcggaacctgtggcgtcatctcgcccactatggaggccagattaagagagttaccagacatgggttacatgttgaacgataagaatggaccacgaggagagttgcaactccgtggttctcaattattcaccaggtactacaagaatccagaagaaactgcaaagtccatcgatgaagacggttggttcagtaccggtgacgttgctgagattggcacagatggctatttcagaattattgacagggtgaagaacttctacaaattatcccagggtgagtatgtttcgccagagaagattgagagtttgtacttgtcgttgaactcgagtattctgcagctttttatccatggggactctaccaagtcatttctcgtgggtgtggtgggtttacagcctgatgttgccagcaagtatgttgatctttcttccggtcccaatgtggtccaagtgttaaaccaacctgagtttagaaagcaattattgttagacctaaacctgaaggtcaatggcaaattgcaagggtttgaaaagttgcacaacattttcatcgacattgaaccattgacactcgagagaaatgttgttaccccaacaatgaagctcaagagacattttgctgccaagtttttcaagccccagatcgaagctatgtatgcagaaggctccattgtcaaagattga

SEQ ID NO: 13 - DNA, 1056 bp, Candida viswanathii
atgtctgataaagcagccgctagagagagattcctctctgtttttgagtgtgccgtcgaggaattgaaagaagtcttggtttctcacaagatgccgcaagaagcaattgactggtttgtcaagaacttgaactacaacacccccggcggtaagttgaacagaggtttgtctgttgtcgacacctacgctatcttgaacaacaccaccgctgacaagttgaacgatgaacaatacaagaaggtcgccttgttgggctggtcaattgaattgttgcaagcttactttttggttgccgatgatatgatggaccaatccaagaccagaagaggacagaaatgttggtacttggtcgaaggtgttggaaacattgcaattaatgactccttcatgttggaaggtgccatttacgtcttgttgaagaagcacttccgtcaagatccatactatgtcgacttgttggacttgttccacgaagtcaccttccagaccgaattgggtcaattattggacttggtgactgctgatgaagaagtcgtcgacttggacaagttctccttggacaagcactcgttcattgtcattttcaaaaccgcatactactccttctacttgcctgttgctttggccatgtacatgagcggtatcagcagcgaagaagacttgaagcaagtcagagatatcttgatcccattgggtgagtacttccaaatccaggacgatttcttggactgtttcggaaccccagaacaaattggcaagatcggtactgatatcaaagacaacaagtgttcctgggtggtcaaccaagctttgttgcatgctactccagaacaacgtaagttgttggacgacaactacggtaagaaagacgacgagtctgaacagagatgcaaggacttgttcaagtccatgggcattgaaaagatctaccacgactacgaagagtcaattgttgctaaattaagagaacaaatcgataaagttgatgaatcaagaggtttgaaaaaagatgtcttgaccgctttcttgggcaaggtttacaagagatccaaatag

SEQ ID NO: 14 - DNA, 125 bp, Artificial Sequence
  Description of Artificial Sequence Synthetic polynucleotide
tgttgcaagcttactggttggttgccgatgatatgatggaccaatccaagaccagaagaggacagaaatgttggtacttggtcgaaggtgttggaaacattgcaatttgggactccttcatgttg

SEQ ID NO: 15 - DNA, 1056 bp, Candida viswanathii
atgtctgataaagcagccgctagagagagattcctctctgtttttgagtgtgccgtcgaggaattgaaagaagtcttggtttctcacaagatgccgcaagaagcaattgactggtttgtcaagaacttgaactacaacacccccggcggtaagttgaacagaggtttgtctgttgtcgacacctacgctatcttgaacaacaccactgctgacaagttgaacgatgaacaatacaagaaggtcgccttgttgggctggtcaattgaattgttgcaagcttactggttggttgccgatgatatgatggaccaatccaagaccagaagaggacagaaatgttggtacttggtcgaaggtgttggaaacattgcaatttgggactccttcatgttggaaggtgccatttacgtcttgttgaagaagcacttccgtcaagatccatactatgtcgacttgttggacttgttccacgaagtcaccttccagaccgaattgggtcaattattggacttggtgactgctgatgaagaagtcgtcgacttggacaagttctccttggacaagcactcgttcattgtcattttcaaaaccgcatactactccttctacttgcctgttgctttggccatgtacatgagcggtatcagcagcgaagaagacttgaagcaagtcagagatatcttgatcccattgggtgagtacttccaaatccaggacgatttcttggactgtttcggaaccccagaacaaattggcaagatcggtactgatatcaaagacaacaagtgttcctgggtggtcaaccaagctttgttgcatgctactccagaacaacgtaagttgttggacgacaactacggtaagaaagacgacgagtctgaacagagatgcaaggacttgttcaagtccatgggcattgaaaagatctaccacgactacgaagagtcaattgttgctaaattaagagaacaaatcgataaagttgatgaatcaagaggtttgaaaaaagatgtcttgaccgctttcttgggcaaggtttacaagagatccaaatag

SEQ ID NO: 16 - DNA, 969 bp, Cannabis sativa
atggctggcagcgaccaaatcgaaggctcgccacaccacgaatcggacaactccattgctaccaagatccttaacttcggtcacacctgctggaagttacaaagaccttacgttgttaaggggatgatctccatagcctgcggcttgttcgggagagagttgttcaacaatagacaccttttctcttggggcttgatgtggaaagccttcttcgccttggtaccaatcttgagtttcaacttcttcgctgctatcatgaatcaaatctatgacgtagacatagatcgtatcaacaaaccagacctacctctagtctccggggaaatgtccatcgaaactgcctggatcttgagcatcatcgtagctctaaccgggttgattgttaccatcaagttgaagtcggctcctttgttcgtcttcatctatatcttcggcatcttcgccgggttcgcgtactcggtccctcctatcagatggaaacaatacccattcacaaacttcctaatcacgattagttctcacgtaggacttgcctttacttcctactcggctacaacttccgccttaggccttcctttcgtatggcgtccagccttctcgttcataattgccttcatgactgtcatgggcatgaccatcgccttcgcgaaggacatctccgacatcgaaggagatgcgaagtacggggtttccaccgtcgctacaaagcttggcgcccggaacatgactttcgtcgtctcgggtgtccttcttctcaactacttggtctcgatttcgatcgggatcatctggccacaggtcttcaaatcgaacatcatgattctttcgcacgctatacttgctttctgtctcatctttcagacccgggagttagccctagctaactatgcgtcagcacctagcagacagttctttgagttcatatggttgctctactacgccgaatacttcgtttacgttttcatttga

SEQ ID NO: 17 - DNA, 924 bp, Streptomyces sp.
atgagtgaagctgccgatgttgagcgagtttacgccgctatggaggaagctgcaggacttttaggggtagcctgtgctcgtgataagatttaccctttattatcaaccttccaagacacattagtggagggtgggtctgtagttgttttctcaatggctagtggacgacactctactgagttggatttcagtatatccgtgcctacatcacacggggacccatacgccactgtagtagagaaaggactttttcctgcaacaggacacccagttgatgatcttcttgccgatacccagaaacaccttcctgtatccatgtttgctatcgacggggaggtgacaggtgggtttaagaagacttatgccttttttccaaccgacaatatgcctggagttgctgagcttagtgctatcccatccatgccacctgctgtggctgagaacgccgagcttttcgctcgttacggtcttgataaggttcaaatgacctcaatggactacaagaagcgtcaggtgaacctttacttctcagagctttcagcccaaacattggaggcagagagtgtattggcccttgttagagagttaggacttcacgtaccaaatgagttaggtttgaaattttgcaaaagatccttctccgtttaccctacattaaactgggagaccggtaagattgatcgtttgtgctttgctgttatcagtaacgacccaactttggtgccttcaagtgatgagggtgatatcgagaagtttcacaattatgcaacaaaagctccttatgcctacgttggggagaagcgaactttggtttatggtcttactctttccccaaaggaagaatattataagctttcagccgcttatcatataactgatgtgcaacgagggcttcttaaggcttttgatagtttggaagactaa

SEQ ID NO: 18 - DNA, 1557 bp, Cannabis sativa
atgaaccctcaggaaaattttttaaagtgcttttccgagtatatcccaaataaccctgctaatcctaagttcatttacactcaacacgatcagttgtatatgtcagttttgaactccacaattcaaaacttaagattcacttccgatactaccccaaagcctttggttatagtaacaccttccaatgtctctcacatccaggcttcaatactttgctccaaaaaggtagggcttcagatacgtactagatctggaggacacgacgcagaaggattgtcttatatatcacaggttccatttgctatcgtagacttgagaaacatgcacacagttaaggttgatattcactcacaaactgcatgggtggaggctggtgctactttgggtgaggtatactattggattaacgaaatgaatgaaaacttctccttccctggaggatattgtcctactgtaggggtaggtgggcatttctctggtggggggtatggggcattgatgcgaaactatggtttggctgccgacaatataatagatgcacaccttgtgaatgtagatgggaaagttttagatcgaaagtccatgggggaggatttattctgggcaatacgagggggagggggtgaaaacttcgggatcatcgcagcctgcaagatcaagttggttgtcgtcccatctaaggctacaatattctcagtgaagaagaatatggagatacatggtcttgtaaaactttttaataaatggcagaatatcgcatacaagtacgacaaggatttaatgttgacaacccattttcgtactcgtaatattactgataatcatgggaaaaataagactacagtccacgggtatttcagtagtatattcttaggaggtgttgacagtttagttgacttgatgaacaagtcatttccagaattaggaatcaagaagactgattgcaaagagctttcttggatcgacacaaccatcttctactctggggtcgtaaattataatactgcaaatttcaagaaagaaatattattggatcgatcagccggtaaaaagacagcatttagtattaaacttgattacgttaaaaaacttattccagaaaccgctatggttaaaattttagaaaaattgtacgaggaagaggtgggtgtcgggatgtacgttctttatccatatggtggtattatggatgaaatatccgagtctgcaatcccatttccacatagagcaggaataatgtatgaactttggtacaccgcaacctgggaaaaacaagaggataatgaaaagcacattaattgggtcagatccgtttataactttactaccccatacgtatcacaaaacccacgtcttgcctatttaaactatagagacttagatcttggtaagacaaatccagagtctcctaataactatactcaagcacgtatctggggagagaagtattttggtaagaattttaatagattagtcaaggttaaaacaaaggctgaccctaacaatttctttagaaacgagcagtccatcccaccacttccaccaagacaccattga

SEQ ID NO: 19 - protein, 706 aa, Arthrobacter ureafaciens
MTEVVDRASSPASPGSTTAAADGAKVAVEPRVDVAALGEQLLGRWADIRLHARDLAGREVVQKVEGLTHTEHRSRVFGQLKYLVDNNAVHRAFPSRLGGSDDHGGNIAGFEELVTADPSLQIKAGVQWGLFGSAVMHLGTREHHDKWLPGIMSLEIPGCFAMTETGHGSDVASIATTATYDEETQEFVIDTPFRAAWKDYIGNAANDGLAAVVFAQLITRKVNHGVHAFYVDLRDPATGDFLPGIGGEDDGIKGGLNGIDNGRLHFTNVRIPRTNLLNRYGDVAVDGTYSSTIESPGRRFFTMLGTLVQGRVSLDGAAVAASKVALQSAIHYAAERRQFNATSPTEEEVLLDYQRHQRRLFTRLATTYAASFAHEQLLQKFDDVFSGAHDTDADRQDLETLAAALKPLSTWHALDTLQECREACGGAGFLIENRFASLRADLDVYVTFEGDNTVLLQLVAKRLLADYAKEFRGANFGVLARYVVDQAAGVALHRTGLRQVAQFVADSGSVQKSALALRDEEGQRTLLTDRVQSMVAEVGAALKGAGKLPQHQAAALFNQHQNELIEAAQAHAELLQWEAFTEALAKVDDAGTKEVLTRLRDLFGLSLIEKHLSWYLMNGRLSMQRGRTVGTYINRLLVKIRPHALDLVDAFGYGAEHLRAAIATGAEATRQDEARTYFRQQRASGSAPADEKTLLAIKAGKSRAKL

SEQ ID NO: 20 - protein, 391 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKYHHHHHH

SEQ ID NO: 21 - protein, 107 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRKHHHHHH

SEQ ID NO: 22 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 23 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 24 - protein, 395 aa, Cannabis sativa
MGLSSVCTFSFQTNYHTLLNPHNNNPKTSLLCYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 25 - protein, 321 aa, Cannabis sativa
MAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 26 - protein, 517 aa, Cannabis sativa
MNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 27 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 28 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 29 - protein, 740 aa, Candida viswanathii
MTTLPSIADTDNIYASDDKEYIFDNPNDLSIETLVNHILPFPQEVAGESVKVPGTAVQGFSEFYRNAATPNGIKLSLIKGLDTYHHIFESSAERYADDPCLAFHEYDYENSQHLERYATISYKEVRQRKDDFAAGLFFLLKANPYKNDSLEAHQKIVNHEANYKSYDSDNMSFIVTFYAANRVEWVLSDLACSSNSITSTALYDTLGPDTSKYILETTESPVIISSKDHIRDLIDLKKANPKELAALILIISMDPLKKSDQNLVHLAEANNIKLYDFSQVERTGAIFPHQTNAPNSETVFTITFTSGTTGANPKGVVLPQRCAASGMLAYSVMMPHHRGTREFAFLPLAHIFERQMVASMFMFGGSSAMPRLGGTPLTLVEDLKLWKPTFMANVPRVFTKIEAGIKASTIDSTSSLTRSLYERAIEAKRVKQNKNDDSGDHFIYDKLLIQRLRSAIGYDCLEFCVTGSAPIAPETIKFLKASLGIGFGQGYGSSESFAGMLFALPFKNSSVGTCGVISPTMEARLRELPDMGYMLNDKNGPRGELQLRGSQLFTRYYKNPEETAKSIDEDGWFSTGDVAEIGTDGYFRIIDRVKNFYKLSQGEYVSPEKIESLYLSLNSSISQLFIHGDSTKSFLVGVVGLQPDVASKYVDLSSGPNVVQVLNQPEFRKQLLLDLNSKVNGKLQGFEKLHNIFIDIEPLTLERNVVTPTMKLKRHFAAKFFKPQIEAMYAEGSIVKDYKL

SEQ ID NO: 30 - protein, 737 aa, Candida viswanathii
MTTLPSIADTDNIYASDDKEYIFDNPNDLSIETLVNHILPFPQEVAGESVKVPGTAVQGFSEFYRNAATPNGIKLSLIKGLDTYHHIFESSAERYADDPCLAFHEYDYENSQHLERYATISYKEVRQRKDDFAAGLFFLLKANPYKNDSLEAHQKIVNHEANYKSYDSDNMSFIVTFYAANRVEWVLSDLACSSNSITSTALYDTLGPDTSKYILETTESPVIISSKDHIRDLIDLKKANPKELAALILIISMDPLKKSDQNLVHLAEANNIKLYDFSQVERTGAIFPHQTNAPNSETVFTITFTSGTTGANPKGVVLPQRCAASGMLAYSVMMPHHRGTREFAFLPLAHIFERQMVASMFMFGGSSAMPRLGGTPLTLVEDLKLWKPTFMANVPRVFTKIEAGIKASTIDSTSSLTRSLYERAIEAKRVKQNKNDDSGDHFIYDKLLIQRLRSAIGYDCLEFCVTGSAPIAPETIKFLKASLGIGFGQGYGSSESFAGMLFALPFKNSSVGTCGVISPTMEARLRELPDMGYMLNDKNGPRGELQLRGSQLFTRYYKNPEETAKSIDEDGWFSTGDVAEIGTDGYFRIIDRVKNFYKLSQGEYVSPEKIESLYLSLNSSISQLFIHGDSTKSFLVGVVGLQPDVASKYVDLSSGPNVVQVLNQPEFRKQLLLDLNSKVNGKLQGFEKLHNIFIDIEPLTLERNVVTPTMKLKRHFAAKFFKPQIEAMYAEGSIVKD

SEQ ID NO: 31 - protein, 351 aa, Candida viswanathii
MSDKAAARERFLSVFECAVEELKEVLVSHKMPQEAIDWFVKNLNYNTPGGKLNRGLSVVDTYAILNNTTADKLNDEQYKKVALLGWSIELLQAYFLVADDMMDQSKTRRGQKCWYLVEGVGNIAINDSFMLEGAIYVLLKKHFRQDPYYVDLLDLFHEVTFQTELGQLLDLVTADEEVVDLDKFSLDKHSFIVIFKTAYYSFYLPVALAMYMSGISSEEDLKQVRDILIPLGEYFQIQDDFLDCFGTPEQIGKIGTDIKDNKCSWVVNQALLHATPEQRKLLDDNYGKKDDESEQRCKDLFKSMGIEKIYHDYEESIVAKLREQIDKVDESRGLKKDVLTAFLGKVYKRSK

SEQ ID NO: 32 - protein, 351 aa, Candida viswanathii
MSDKAAARERFLSVFECAVEELKEVLVSHKMPQEAIDWFVKNLNYNTPGGKLNRGLSVVDTYAILNNTTADKLNDEQYKKVALLGWSIELLQAYWLVADDMMDQSKTRRGQKCWYLVEGVGNIAIWDSFMLEGAIYVLLKKHFRQDPYYVDLLDLFHEVTFQTELGQLLDLVTADEEVVDLDKFSLDKHSFIVIFKTAYYSFYLPVALAMYMSGISSEEDLKQVRDILIPLGEYFQIQDDFLDCFGTPEQIGKIGTDIKDNKCSWVVNQALLHATPEQRKLLDDNYGKKDDESEQRCKDLFKSMGIEKIYHDYEESIVAKLREQIDKVDESRGLKKDVLTAFLGKVYKRSK

SEQ ID NO: 33 - protein, 322 aa, Cannabis sativa
MAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 34 - protein, 307 aa, Streptomyces sp.
MSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLSAAYHITDVQRGLLKAFDSLED

SEQ ID NO: 35 - protein, 518 aa, Cannabis sativa
MNPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAACKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 36 - DNA, 21 bp, Artificial Sequence
  Description of Artificial Sequence Synthetic oligonucleotide
ttccgcttaatggagtccaaa

SEQ ID NO: 37 - DNA, 20 bp, Artificial Sequence
  Description of Artificial Sequence Synthetic oligonucleotide
taaacgttgggcaaccttgg

SEQ ID NO: 38 - DNA, 37 bp, Artificial Sequence
  Description of Artificial Sequence Synthetic oligonucleotide
cacacagctcttcagccatgacagaagttgttgatag

SEQ ID NO: 39 - DNA, 29 bp, Artificial Sequence
  Description of Artificial Sequence Synthetic oligonucleotide
atgacagaagttgttgatagagcctcatc

SEQ ID NO: 40 - DNA, 22 bp, Artificial Sequence
  Description of Artificial Sequence Synthetic oligonucleotide
tcttgacttaccggccttgatg

SEQ ID NO: 41 - DNA, 49 bp, Artificial Sequence
  Description of Artificial Sequence Synthetic oligonucleotide
cacacagctcttcgagcctacaacttggctcttgacttaccggccttga

SEQ ID NO: 42 - DNA, 13 bp, Artificial Sequence
  Description of Artificial Sequence Synthetic oligonucleotide
taggttaattaaa

SEQ ID NO: 43 - DNA, 15 bp, Artificial Sequence
  Description of Artificial Sequence Synthetic oligonucleotide
agcttttaattaacc

SEQ ID NO: 44 - DNA, 20 bp, Artificial Sequence
  Description of Artificial Sequence Synthetic oligonucleotide
ctctggttctggtgtctttc

SEQ ID NO: 45 - DNA, 20 bp, Artificial Sequence
  Description of Artificial Sequence Synthetic oligonucleotide
gccggcataacatataagtc

SEQ ID NO: 46 - DNA, 27 bp, Artificial Sequence
  Description of Artificial Sequence Synthetic oligonucleotide
gattgattgttatagtttctttctttc

SEQ ID NO: 47 - DNA, 23 bp, Artificial Sequence
  Description of Artificial Sequence Synthetic oligonucleotide
gagtgactcttttgataagagtc

SEQ ID NO: 48 - DNA, 20 bp, Artificial Sequence
  Description of Artificial Sequence Synthetic oligonucleotide
tgaccccctatcgctacggt

SEQ ID NO: 49 - DNA, 20 bp, Artificial Sequence
  Description of Artificial Sequence Synthetic oligonucleotide
atggggaggaggacgaggaa

SEQ ID NO: 50 - DNA, 42 bp, Artificial Sequence
  Description of Artificial Sequence Synthetic oligonucleotide
ccacgtcggtaccgagatcgttgccgaggcaatcaagtcctt

SEQ ID NO: 51 - DNA, 50 bp, Artificial Sequence
  Description of Artificial Sequence Synthetic oligonucleotide
aacggcttcgtctaaacaaccacggatcttcaacaatccctgttctggac

SEQ ID NO: 52 - DNA, 50 bp, Artificial Sequence
  Description of Artificial Sequence Synthetic oligonucleotide
gtccagaacagggattgttgaagatccgtggttgtttagacgaagccgtt

SEQ ID NO: 53 - DNA, 45 bp, Artificial Sequence
  Description of Artificial Sequence Synthetic oligonucleotide
agtgtttgtgtccggtaacgaccgaaatattacaattggagctcc

SEQ ID NO: 54 - DNA, 45 bp, Artificial Sequence
  Description of Artificial Sequence Synthetic oligonucleotide
ggagctccaattgtaatatttcggtcgttaccggacacaaacact

SEQ ID NO: 55 - DNA, 19 bp, Artificial Sequence
  Description of Artificial Sequence Synthetic oligonucleotide
tttcagcaacggcatcacc

SEQ ID NO: 56 - DNA, 22 bp, Artificial Sequence
  Description of Artificial Sequence Synthetic oligonucleotide
acctttatgccaacatcagacc

SEQ ID NO: 57 - DNA, 47 bp, Artificial Sequence
  Description of Artificial Sequence Synthetic oligonucleotide
aacggcttcgtctaaacaacccatcaacggtgtacttttcagtatcc

SEQ ID NO: 58 - DNA, 47 bp, Artificial Sequence
  Description of Artificial Sequence Synthetic oligonucleotide
ggatactgaaaagtacaccgttgatgggttgtttagacgaagccgtt

SEQ ID NO: 59 - DNA, 42 bp, Artificial Sequence
  Description of Artificial Sequence Synthetic oligonucleotide
ttgcaatgccatgaacgcccgaaatattacaattggagctcc

SEQ ID NO: 60 - DNA, 42 bp, Artificial Sequence
  Description of Artificial Sequence Synthetic oligonucleotide
ggagctccaattgtaatatttcgggcgttcatggcattgcaa

SEQ ID NO: 61 - DNA, 20 bp, Artificial Sequence
  Description of Artificial Sequence Synthetic oligonucleotide
cagatggcaacaatcccaag

SEQ ID NO: 62 - DNA, 30 bp, Artificial Sequence
  Description of Artificial Sequence Synthetic oligonucleotide
atcgattaaattctttaattgagggatgtg

SEQ ID NO: 63 - DNA, 38 bp, Artificial Sequence
  Description of Artificial Sequence Synthetic oligonucleotide
gagtgactcttttgataagagtcgcaaatttgatttca

SEQ ID NO: 64 - DNA, 39 bp, Artificial Sequence
  Description of Artificial Sequence Synthetic oligonucleotide
caattaaagaatttaatcgatatgggtttgtcctctgtg

SEQ ID NO: 65 - DNA, 33 bp, Artificial Sequence
  Description of Artificial Sequence Synthetic oligonucleotide
cccttcatctttatcgtgataatcaacccaaag

SEQ ID NO: 66 - DNA, 28 bp, Artificial Sequence
  Description of Artificial Sequence Synthetic oligonucleotide
atcacgataaagatgaagggcggtccat

SEQ ID NO: 67 - DNA, 47 bp, Artificial Sequence
  Description of Artificial Sequence Synthetic oligonucleotide
tcttatcaaaagagtcactctcagataaacacgtaaaccaaatattc

SEQ ID NO: 68 - DNA, 42 bp, Artificial Sequence
  Description of Artificial Sequence Synthetic oligonucleotide
caattaaagaatttaatcgatatgaaccctagagaaaacttc

SEQ ID NO: 69 - DNA, 28 bp, Artificial Sequence
  Description of Artificial Sequence Synthetic oligonucleotide
taatagtatcaatccatgacaactgtcg

SEQ ID NO: 70 - DNA, 37 bp, Artificial Sequence
  Description of Artificial Sequence Synthetic oligonucleotide
gtcatggattgatactattatattttacagtggagtg

SEQ ID NO: 71 - DNA, 38 bp, Artificial Sequence
  Description of Artificial Sequence Synthetic oligonucleotide
tcttatcaaaagagtcactctcagtgtctatgcctagg

SEQ ID NO: 72 - DNA, 46 bp, Artificial Sequence
  Description of Artificial Sequence Synthetic oligonucleotide
gattgattgttatagtttctttctttcttttgaggatgaccagatg

SEQ ID NO: 73 - DNA, 24 bp, Artificial Sequence
  Description of Artificial Sequence Synthetic oligonucleotide
gagtgactcttttgataagagtcg

SEQ ID NO: 74 - DNA, 47 bp, Artificial Sequence
  Description of Artificial Sequence Synthetic oligonucleotide
aagaaagaaactataacaatcaatcatgaaccatttgagggctgaag

SEQ ID NO: 75 - DNA, 50 bp, Artificial Sequence
  Description of Artificial Sequence Synthetic oligonucleotide
gcgactcttatcaaaagagtcactcttaatacttgataggaacgcttctg

SEQ ID NO: 76 - DNA, 47 bp, Artificial Sequence
  Description of Artificial Sequence Synthetic oligonucleotide
aagaaagaaactataacaatcaatcatggccgttaaacacttgatag

SEQ ID NO: 77 - DNA, 46 bp, Artificial Sequence
  Description of Artificial Sequence Synthetic oligonucleotide
gcgactcttatcaaaagagtcactcttatttccgcggcgtataatc

SEQ ID NO: 78 - DNA, 18 bp, Artificial Sequence
  Description of Artificial Sequence Synthetic oligonucleotide
aattaacctatggtgcac

SEQ ID NO: 79 - DNA, 23 bp, Artificial Sequence
  Description of Artificial Sequence Synthetic oligonucleotide
ttaattaaaagcttggcgtaatc

SEQ ID NO: 80 - DNA, 32 bp, Artificial Sequence
  Description of Artificial Sequence Synthetic oligonucleotide
ctcgtgctagtcagtcttgcacgctttgggtg

SEQ ID NO: 81 - DNA, 44 bp, Artificial Sequence
  Description of Artificial Sequence Synthetic oligonucleotide
atgattacgccaagcttttaattaacaacacggcgtctgaggac

SEQ ID NO: 82 - DNA, 45 bp, Artificial Sequence
  Description of Artificial Sequence Synthetic oligonucleotide
actgagagtgcaccataggttaattaactcggggccgtcggtgga

SEQ ID NO: 83 - DNA, 37 bp, Artificial Sequence
  Description of Artificial Sequence Synthetic oligonucleotide
aagcgtgcaagactgactagcacgagcgaagatgggg

SEQ ID NO: 84 - DNA, 19 bp, Artificial Sequence
  Description of Artificial Sequence Synthetic oligonucleotide
gtcttgcacgctttgggtg

SEQ ID NO: 85 - DNA, 18 bp, Artificial Sequence
  Description of Artificial Sequence Synthetic oligonucleotide
tgactagcacgagcgaag

SEQ ID NO: 86 - DNA, 44 bp, Artificial Sequence
  Description of Artificial Sequence Synthetic oligonucleotide
tccccatcttcgctcgtgctagtcaaagggaagaagagtcgttg

SEQ ID NO: 87 - DNA, 44 bp, Artificial Sequence
  Description of Artificial Sequence Synthetic oligonucleotide
cgtcggcacccaaagcgtgcaagacgtcgacctaaattcgcaac

SEQ ID NO: 88 - DNA, 18 bp, Artificial Sequence
  Description of Artificial Sequence Synthetic oligonucleotide
ctcggggccgtcggtgga

SEQ ID NO: 89 - DNA, 23 bp, Artificial Sequence
  Description of Artificial Sequence Synthetic oligonucleotide
caacacggcgtctgaggacttgg

SEQ ID NO: 90 - DNA, 41 bp, Artificial Sequence
  Description of Artificial Sequence Synthetic oligonucleotide
agaaactataacaatcaatcatgaccactttgccttcgatc

SEQ ID NO: 91 - DNA, 45 bp, Artificial Sequence
  Description of Artificial Sequence Synthetic oligonucleotide
tcttatcaaaagagtcactctcacaacttgtaatctttgacaatg

SEQ ID NO: 92 - DNA, 39 bp, Artificial Sequence
  Description of Artificial Sequence Synthetic oligonucleotide
gaaactataacaatcaatcatgggcaagaactacaagag

SEQ ID NO: 93 - DNA, 39 bp, Artificial Sequence
  Description of Artificial Sequence Synthetic oligonucleotide
ttatcaaaagagtcactctcattcgaaatgactaaattg

SEQ ID NO: 94 - DNA, 21 bp, Artificial Sequence
  Description of Artificial Sequence Synthetic oligonucleotide
tgagagtgactcttttgataa

SEQ ID NO: 95 - DNA, 19 bp, Artificial Sequence
  Description of Artificial Sequence Synthetic oligonucleotide
atctttgacaatggagcct

SEQ ID NO: 96 - DNA, 34 bp, Artificial Sequence
  Description of Artificial Sequence Synthetic oligonucleotide
gaatagaagagagtgactcttttgataagagtcg

SEQ ID NO: 97 - DNA, 28 bp, Artificial Sequence
  Description of Artificial Sequence Synthetic oligonucleotide
gattgattgttatagtttctttctttct

SEQ ID NO: 98 - DNA, 21 bp, Artificial Sequence
  Description of Artificial Sequence Synthetic oligonucleotide
gcaatttgggactccttcatg

SEQ ID NO: 99 - DNA, 20 bp, Artificial Sequence
  Description of Artificial Sequence Synthetic oligonucleotide
aaccaaccagtaagcttgca

SEQ ID NO: 100 - DNA, 18 bp, Artificial Sequence
  Description of Artificial Sequence Synthetic oligonucleotide
ctcggggccgtcggtgga

SEQ ID NO: 101 - DNA, 23 bp, Artificial Sequence
  Description of Artificial Sequence Synthetic oligonucleotide
caacacggcgtctgaggacttgg

(19) United States
(12) Patent Application Publication
(10) Pub. No.: US 2022/0186267 A1
(43) Pub. Date: Jun. 16, 2022

(54) Biosynthetic Cannabidiol Production In Engineered Microorganisms

(71) Applicant: Rynetech Bio, Inc., Carlsbad, CA (US)

(72) Inventor: Thomas A. BEARDSLEE, Carlsbad, CA (US)

(73) Assignee: Rynetech Bio, Inc., Carlsbad, CA (US)

(21) Appl. No.: 17/598,863

(22) PCT Filed: Mar. 27, 2020
(86) PCT No.: PCT/US20/25462

§ 371 Date: Sep. 27, 2021

Related U.S. Application Data
(60) Provisional application No. 62/824,615, filed on Mar. 27, 2019.

(51) Int. Cl. C12P 7/42; C12N 1/16; C12N 15/52; C12N 15/81

(52) U.S. Cl. CPC C12P 7/42; C12N 1/16; C12N 15/52; C12N 15/815; C12Y 121/03008; C12Y 103/03006; C12N 2510/02; C12Y 205/0101; C12Y 205/01102; C12Y 404/01026; C12Y 602/01002; C12N 2800/102; C12Y 203/01206

(57) ABSTRACT

The invention provides engineered biosynthetic pathways that can be used to produce cannabinoids from fatty acids, recombinant microorganisms incorporating such pathways, methods of biosynthetically producing cannabinoids from fatty acids, and cannabinoids so produced.

### RELATED APPLICATIONS

This application claims the benefit of and priority to PCT application serial number PCT/US2020/025462 filed 27 Mar. 2020 and published as WO 2020/198679 on 1 Oct. 2020 (attorney docket no. RYN-0100-PC), which claims priority to U.S. provisional patent application Ser. No. 62/824,615, filed 27 Mar. 2019 (attorney docket no. RYN-0100-PV), the contents of which is hereby incorporated by reference in its entirety for any and all purposes.

### SEQUENCE LISTING

The instant application contains a Sequence Listing which has been submitted electronically in ASCII format and is hereby incorporated by reference in its entirety. Said ASCII copy, created on Sep. 24, 2021, is named RYN-0100-US_SL.txt and is 112,638 bytes in size.

### TECHNICAL FIELD OF THE INVENTION

This invention relates to biosynthetic production of cannabinoids and recombinant microorganisms engineered to produce cannabinoids.

### BACKGROUND OF THE INVENTION

### I. Introduction

The following description includes information that may be useful in understanding the present invention. It is not an admission that any such information is prior art, or relevant, to the presently claimed inventions, or that any publication specifically or implicitly referenced is prior art or even particularly relevant to the presently claimed invention.

### II. Background

Microorganisms employ various enzyme-driven biological pathways to support their metabolism and growth. These pathways can be exploited for the production (i.e., biosynthesis) of naturally produced products. The pathways also can be altered via recombinant DNA technology to increase production or to produce different products that may be commercially valuable.

Recently, decriminalization and legalization of marijuana has led to increased interest in the production of various compounds produced by cannabis plants. Among these is cannabidiol (CBD), a phytocannabinoid (or cannabinoid) being studied for the treatment of anxiety, cognition, movement disorders, and pain, among others. CBD lacks the psychoactivity of tetrahydrocannabinol (THC), and may interact with different biological targets, including neurotransmitter receptors such as cannabinoid receptors. Other cannabinoids that are produced at low levels in cannabis plants are also of interest such as cannabigerol (CBG) and cannabichromene (CBC). Accordingly, there is a need to provide suitable sources of cannabinoids.

### III. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings. In the event of conflict, the present specification, including definitions, will control.

As used herein, the singular forms “a”, “an”, and “the” include plural references unless the context clearly dictates otherwise.

The term “about” refers to approximately a +/−10% variation from the stated value. It is to be understood that such a variation is always included in any given value provided herein, whether or not it is specifically referred to.

The term “altered activity” refers to an activity in an engineered microorganism of the invention that is added or modified relative to the host microorganism (e.g., added, increased, reduced, inhibited, or removed activity). An activity can be altered by introducing a genetic modification into a host microorganism that yields an engineered microorganism having added, increased, reduced, inhibited, or removed activity.

The term “beta oxidation pathway” as used herein, refers to a series of enzymatic activities utilized to metabolize fatty alcohols, fatty acids, or dicarboxylic acids. The activities utilized to metabolize fatty alcohols, fatty acids, or dicarboxylic acids include, but are not limited to, acyl-CoA ligase activity, acyl-CoA oxidase activity, acyl-CoA dehydrogenase activity, acyl-CoA hydrolase activity, acyl-CoA thioesterase activity, enoyl-CoA hydratase activity, 3-hydroxyacyl-CoA dehydrogenase activity and acetyl-CoA C-acyltransferase activity. The term “beta oxidation activity” refers to any of the activities in the beta oxidation pathway utilized to metabolize fatty alcohols, fatty acids or dicarboxylic acids.

The term “genetic modification” refers to any suitable nucleic acid addition, removal, or alteration that facilitates production of a desired product (or intermediate) in an engineered microorganism. Genetic modifications include insertion, deletion, modification, or substitution of one or more nucleotides in a native nucleic acid of a host organism in one or more locations, insertion of a non-native nucleic acid into a host microorganism (e.g., insertion of an autonomously replicating vector or plasmid), and removal of a non-native nucleic acid in a host microorganism (e.g., removal of a vector).

The term “heterologous polynucleotide” refers to a nucleotide sequence not present in a host microorganism in some embodiments. In certain embodiments, a heterologous polynucleotide is present in a different amount (e.g., different copy number) than in the parent microorganism, which can be accomplished, for example, by introducing more copies of a particular nucleotide sequence into a host microorganism (e.g., the particular nucleotide sequence may be in a nucleic acid autonomous of the host chromosome (e.g., a plasmid) or may be inserted into a chromosome). A heterologous polynucleotide is from a different organism in some embodiments, and in certain embodiments, is from the same type of organism but from an outside source (e.g., a recombinant source).

A “patentable” composition, machine, method, process, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

A “plurality” means more than one.

The term “species”, when used in the context of describing a particular compound or molecule species, refers to a population of chemically indistinct molecules.

Where a range of values is provided in this specification, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

### SUMMARY OF THE INVENTION

The present invention addresses the need for improved sources of cannabinoids, one of which is cannabidiol (CBD).

In one aspect, the invention concerns recombinant microorganism engineered to biosynthetically produce cannabidiol from fatty acids. Such microorganisms include a first engineered biosynthetic pathway to produce hexanoyl-CoA via degradation of fatty acids, preferably monounsaturated fatty acids, either supplied exogenously or via de novo fatty acid synthesis and a second engineered biosynthetic pathway to produce cannabidiolic acid from hexanoyl-CoA. The cannabidiolic acid so produced then typically non-enzymatically decarboxylates to yield cannabidiol, which can then be isolated.

Preferred recombinant microorganisms include recombinant fungi (e.g., Aspergillus, Thraustochytrium, Rhizopus, and Schizochytrium fungi) and yeast such as various Candida species, particularly C. revkaufi, C. viswanathii, C. pulcherrima, C. tropicalis, C. utilis, as well as yeast from other yeast genera, including, Arachniotus, Aspergillus, Aureobasidium, Auxarthron, Blastobotrys, Blastomyces, Candida, Chrysosporuim, Chrysosporuim Debaryomyces, Coccidiodes, Cryptococcus, Gymnoascus, Hansenula, Histoplasma, Issatchenkia, Kluyveromyces, Lipomyces, Lssatchenkia, Microsporum, Myxotrichum, Myxozyma, Oidiodendron, Pacysolen, Penicillium, Pichia, Rhodosporidium, Rhodotorula, Rhodoturala, Saccharomyces, Schizosaccharomyces, Scopulariopsis, Sepedonium, Trichosporon, and Yarrowia.

In certain preferred embodiments, a recombinant microorganism of the invention includes a heterologous fatty acyl-CoA oxidase gene, a heterologous fatty acyl-CoA synthetase gene, a heterologous polyketide synthase (PKS) or tetraketide synthase (TKS) gene, a heterologous olivetolic acid cyclase (OAC) gene, a heterologous aromatic prenyltransferase (GOT) gene, and a heterologous cannabidiolic acid synthase (CBDAS) gene.

In other preferred embodiments, a recombinant microorganism of the invention includes a heterologous fatty acyl-CoA oxidase gene, a heterologous fatty acyl-CoA synthetase gene, a heterologous polyketide synthase (PKS) or tetraketide synthase (TKS) gene, a heterologous olivetolic acid cyclase (OAC) gene, a heterologous aromatic prenyltransferase (GOT) gene, and a heterologous cannabichromenic acid synthase (CBCAS) gene.

In other preferred embodiments, a recombinant microorganism of the invention includes a heterologous fatty acyl-CoA oxidase gene, a heterologous fatty acyl-CoA synthetase gene, a heterologous polyketide synthase (PKS) or tetraketide synthase (TKS) gene, a heterologous olivetolic acid cyclase (OAC) gene, a heterologous aromatic prenyltransferase (GOT) gene, and a heterologous tetrahydrocannabinolic acid synthase (THCAS) gene.

In still other preferred embodiments, a recombinant microorganism of the invention includes a heterologous synthase gene (other than CBDAS or CBCAS or THCAS) for producing other cannabinoids from a cannabigerolic acid precursor.

A related aspect concerns biosynthetic cannabinoid production methods. Such methods generally involve cultivating a recombinant microorganism according to the invention in a feedstock that includes a carbon source, optionally glycerol or one or more fatty acid species, under growth conditions that promote production of a cannabinoid, and recovering the cannabinoid following non-enzymatic decarboxylation of said cannabinoid. In preferred embodiments, such methods cannabinoid yield is about 0.001 g/L to about 100 g/L.

More specifically, a related aspect concerns biosynthetic cannabidiol production methods. Such methods generally involve cultivating a recombinant microorganism according to the invention in a feedstock that includes a carbon source, optionally glycerol or one or more fatty acid species, under growth conditions that promote production of cannabidiolic acid, and recovering cannabidiol following non-enzymatic decarboxylation of cannabidiolic acid. In preferred embodiments, such methods cannabidiol yield is about 0.001 g/L to about 100 g/L.

The biosynthetic methods of the invention may have significantly less environmental impact and are economically competitive with current cannabinoid manufacturing systems.

These and other aspects and embodiments of the invention are discussed in greater detail in the sections that follow.

### BRIEF DESCRIPTION OF THE FIGURES

FIG. 1: Overview of the biochemical pathway for the production of cannabidiolic acid in engineered yeast. Multi-step pathways are indicated with boxes while individual pathway enzymes are indicated by ovals. Shading indicates pathways and steps that are potential targets for engineering in the pathway.

FIG. 2: Pathway detail for peroxisomal beta-oxidation in yeast. Shading indicates steps that are potential targets for engineering in the pathway.

FIG. 3: Plasmid map for plasmid pAA244 showing the URA3 marker with terminator repeats.

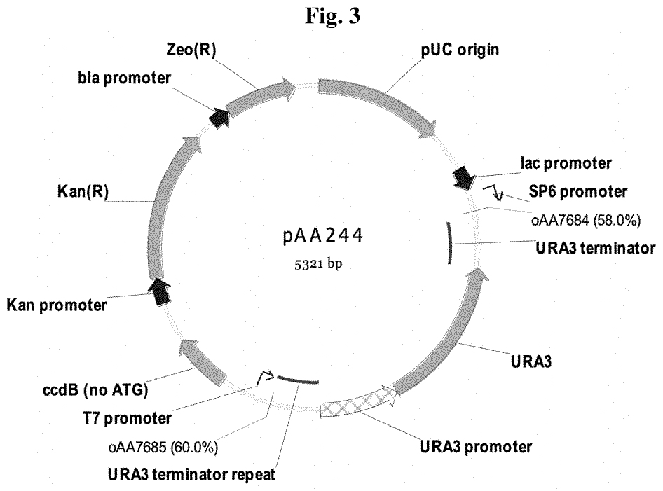

FIG. 4: Plasmid map for plasmid pAA335. Genes of interest may be inserted between the PEX11 promoter and terminator by restriction cloning with BspQI or other methods.

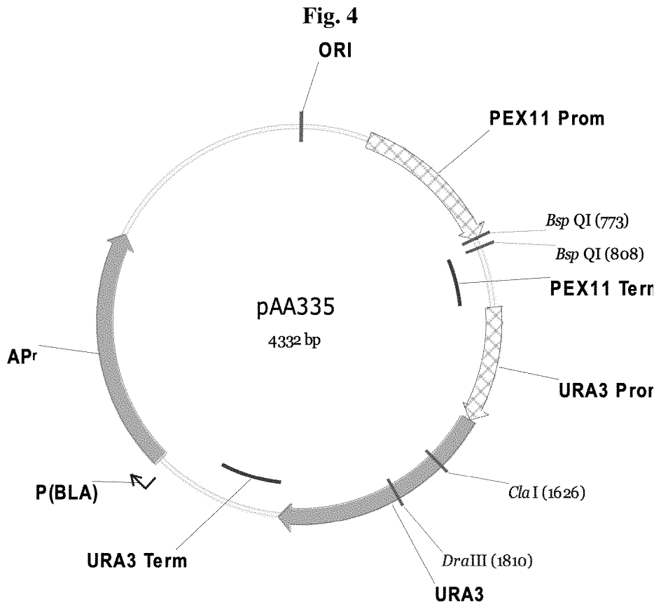

FIG. 5: Plasmid map for plasmid pAA1164 containing a split URA3 marker, HDE promoter, and PDX4 terminator. HDE and FOX2 are synonymous in the art and may be used interchangeably.

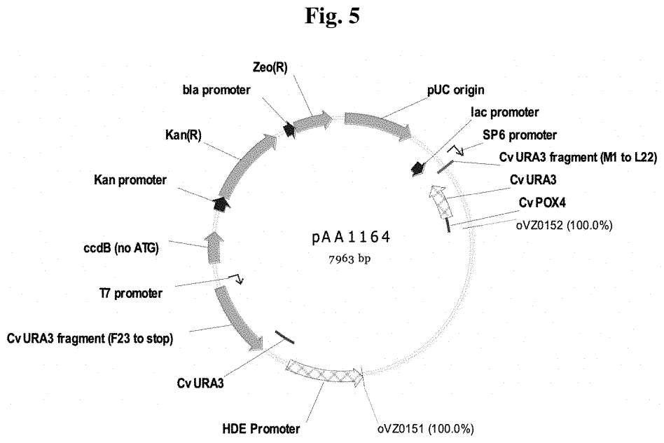

FIG. 6: Plasmid map for plasmid pVZ4045 containing a split LEU2 marker ready for insertion of promoter-gene-terminator sequence between the LEU2 terminator and LEU2 promoter.

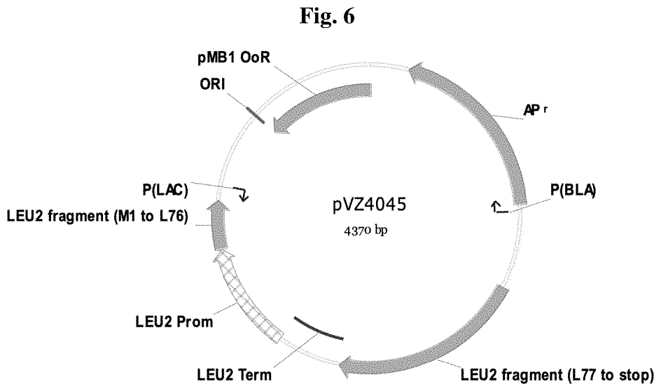

FIG. 7A-FIG. 7C: a) Gas chromatography mass spectrometry total ion chromatogram for shake flask samples from strain sAA9712 with (gray line) and without (black line) hexanoic acid supplementation. The peak indicated at retention time 4.17 minutes is olivetolic acid. b) Fragment ion spectra for olivetolic acid standard. c) Fragment ion spectra of 4.17 minute peak for shake flask sample from strain sAA9712 supplemented with hexanoic acid matching the olivetolic acid standard.

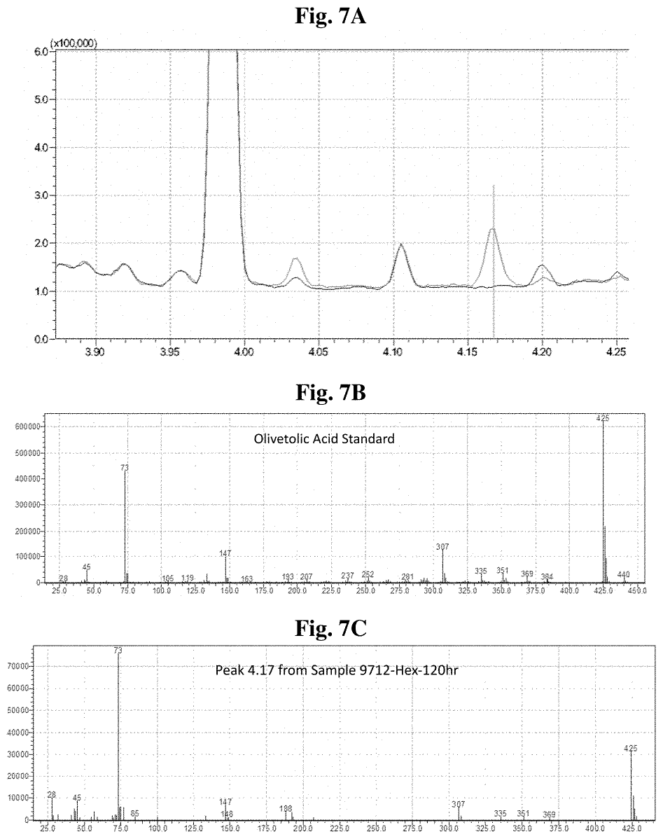

FIG. 8: Gas chromatography mass spectrometry total ion chromatogram and fragment ion spectra for shake flask samples from yeast strains fed oleic acid. Ion spectra for the retention time 4.17 minute peak match the olivetolic acid standard spectra.

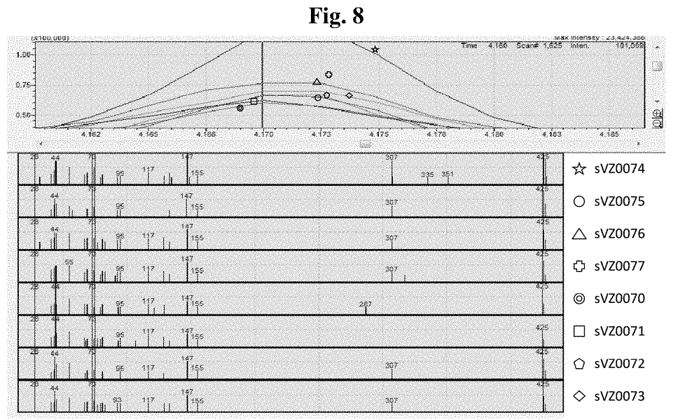

FIG. 9: Gas chromatography mass spectrometry total ion chromatogram and fragment ion spectra for shake flask samples from yeast strains fed oleic acid and with (indicated by -G) or without glycerol. Ion spectra for the retention time 4.17 minute peak match the olivetolic acid standard spectra. Total ion count for samples fed glycerol are increased over samples without glycerol.

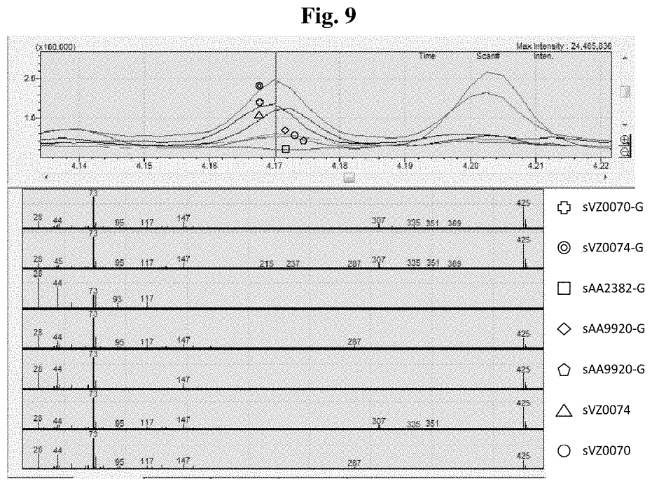

FIG. 10: Plasmid map for plasmid pVZ4285 containing a split LEU2 marker, HDE promoter, and PDX4 terminator. HDE and FOX2 are synonymous in the art and may be used interchangeably. Oligonucleotides shown on the map were for vector construction (oRB001/oRB002) or for PCR amplification of transformation cassettes (oRB010/oRB011).

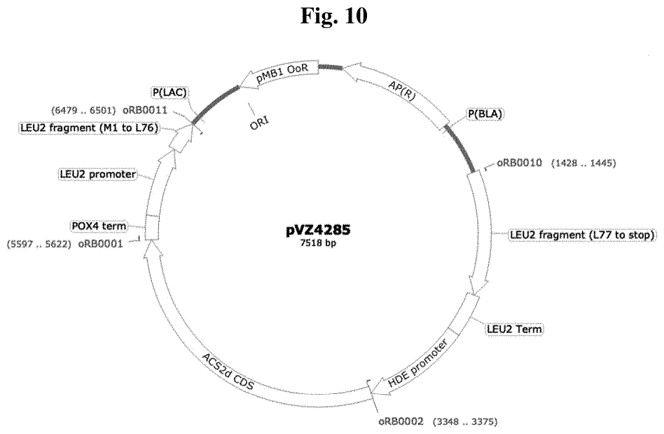

### DETAILED DESCRIPTION OF THE INVENTION

As described herein, this invention concerns microorganisms engineered to produce various cannabinoid species. One such cannabinoid is cannabidiol (C₂₁H₃₀O₂, 314.46 g/mol, melting point (mp) 66° C., boiling point (bp) 180° C.; CBD), a non-psychoactive cannabinoid naturally produced in Cannabis sativa in trichomes (structures on female flowers). CBD has neuroprotective properties and may find applications in treating epilepsy and other conditions. The chemical structure of CBD is shown below.

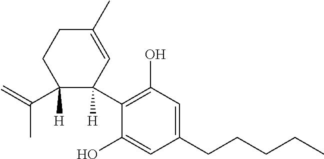 [SMILES: C=C(C)[C@@H]1CCC(C)=C[C@H]1c1c(O)cc(CCCCC)cc1O]

### CBD Pathway

CBD is produced via the decarboxylation of cannabidiolic acid that occurs spontaneously upon heating, making the fermentation objective cannabidiolic acid (CBDA). As shown in FIG. 0, the pathway begins with hexanoyl-CoA, which is a substrate for a polyketide synthase (PKS, this enzyme may also be referenced as a tetraketide synthase or TKS). PKS processes three malonyl-CoA's to produce a 12-carbon molecule 3,5,7-trioxododecanoyl-CoA that is in turn converted to olivetolic acid (OA) by the enzyme olivetolic acid cyclase (OAC). Without the OAC cyclase enzyme, PKS produces olivetol from 3,5,7-trioxododecanoyl-CoA, which is not a substrate for the next reaction in the pathway. In that next step, olivetolic acid and geranyl pyrophosphate (GPP) are combined by an aromatic prenyltransferase to produce cannabigerolic acid, which can be converted either to THCA or CBDA. The enzyme responsible for producing the CBDA is cannabidiolic acid synthase (CBDA synthase).

### Microorganisms

While yeast represent a preferred class of microorganisms that can be engineered in accordance with the invention to biosynthetically produce cannabinoids such as cannabidiol (CBD), the invention includes engineering any suitable microorganism for this purpose. A microorganism selected for biosynthetic CBD production in accordance with the invention will be suitable for genetic manipulation and often can be cultured at cell densities useful for industrial production of CBD in a fermentation device. Here, the term “engineered microorganism” refers to a modified microorganism that includes one or more activities distinct from an activity present in the microorganism utilized as a starting point (hereafter a “host” or “parent” microorganism). An engineered microorganism typically includes at least one heterologous polynucleotide. Thus, an engineered microorganism is one that has been altered directly or indirectly by a human being to achieve a desired objective, for example, CBD production. A host microorganism sometimes is a native microorganism, and at times is a microorganism that has been previously engineered to a certain point.

Preferably, an engineered microorganism is a single cell organism, often capable of dividing and proliferating. A microorganism can include one or more of the following features: aerobe, anaerobe, filamentous, non-filamentous, monoploid, dipoid, auxotrophic, and/or non-auxotrophic. In certain embodiments, an engineered microorganism is a prokaryotic microorganism (e.g., a bacterium), and in certain embodiments, an engineered microorganism is a eukaryotic microorganism (e.g., yeast, fungi, amoeba).

Particularly preferred parent microorganisms (and source for heterologous or modified polynucleotides) are any suitable yeast, including Yarrowia yeast (e.g., Y. lipolytica (formerly classified as: Candida lipolytia)), Candida yeast (e.g., C. revkaufi, C. viswanathii, C. pulcherrima, C. tropicalis, C. utilis), Rhodotorula yeast (e.g., R. glutinus, R. graminis), Rhodosporidium yeast (e.g., R. toruloides), Saccharomyces yeast (e.g., S. cerevisiae, S. bayanus, S. pastorianus, S. carlsbergensis), Cryptococcus yeast, Trichosporon yeast (e.g., T. pullans, T. cutancum), Pichia yeast (e.g., P. pastoris), Blastobotrys yeast (e.g., B. adeninivorans (formerly classified as: Arxula adeninivorans)), and Lipomyces yeast (e.g., L. starkeyii, L. lipoferus). In some embodiments, a suitable yeast is of the genus Arachniotus, Aspergillus, Aureobasidium, Auxarthron, Blastobotrys, Blastomyces, Candida, Chrysosporuim, Chrysosporuim Debaryomyces, Coccidiodes, Cryptococcus, Gymnoascus, Hansenula, Histoplasma, Issatchenkia, Kluyveromyces, Lipomyces, Lssatchenkia, Microsporum, Myxotrichum, Myxozyma, Oidiodendron, Pacysolen, Penicillium, Pichia, Rhodosporidium, Rhodotorula, Rhodoturala, Saccharomyces, Schizosaccharomyces, Scopulariopsis, Sepedonium, Trichosporon, or Yarrowia. In some embodiments, a suitable yeast is of the species Arachniotus flavolutcus, Aspergillus flavus, Aspergillus furnigatus, Aspergillus niger, Aurcobasidium pullulans, Auxarthron thaxteri, Blastobotrys adeninivorans, Blastomyces dermatitidis, Candida albicans, Candida dubliniensis, Candida famata, Candida glabrata, Candida guilliermondii, Candida kefyr, Candida krusei, Candida lambica, Candida lipolytica, Candida lustitaniae, Candida parapsilosis, Candida pulcherrima, Candida revkaufi, Candida rugosa, Candida tropicalis, Candida utilis, Candida viswanathii, Candida xestobii, Chrysosporuim keratinophilum, Coccidiodes immitis, Cryptococcus albidus var. diffluens, Cryptococcus laurentii, Cryptococcus neoformans, Debaryomyces hansenii, Gymnoscus dugwayensis, Hansenula anomala, Histoplasma capsulatum, Issatchenkia occidentalis, lsstachenkia orientalis, Kluyveromyces lactis, Kluyveromyces marxianus, Kluyveromyces thermotolerans, Kluyveromyces waltii, Lipomyces lipoferus, Lipomyces starkeyii, Microsporum gypseum, Myxotrichum deflexum, Oidiodendron echinulatum, Pachysolen tannophilis, Penicillium notatum, Pichia anomala, Pichia pastoris, Pichia stipitis, Rhodosporidium toruloides, Rhodotorula glutinus, Rhodotorula graminis, Saccharomyces cerevisiae, Saccharomyces kluyveri, Schizosaccharomyces pombe, Scopulariopsis acremonium, Sepedonium chrysospermum, Trichosporon cutancum, Trichosporon pullas, Yarrowia lipolytica, or Yarrowia lipolytica (formerly classified as Candida lipolytica). In certain preferred embodiments, the yeast is a Candida species (i.e., Candida spp.), including, Candida albicans, Candida dubliniensis, Candida famata, Candida glabrata, Candida guilliermondii, Candida kefyr, Candida krusei, Candida lambica, Candida lipolytica, Candida lustitaniae, Candida parapsilosis, Candida pulcherrima, Candida revkaufi, Candida rugosa, Candida tropicalis, Candida utilis, Candida viswanathii, Candida xestobii, etc.

Any suitable fungus can be selected as a host microorganism or source for a heterologous polynucleotide. Non-limiting examples of fungi include Aspergillus fungi (e.g., A. parasiticus, A. nidulans), Thraustochytrium fungi, Schizochytrium fungi and Rhizopus fungi (e.g., R. arrhizus, R. oryzae, R. nigricans).

Any suitable prokaryote can also be selected as a host microorganism or source for a heterologous polynucleotide, including Gram negative and Gram positive bacteria. Examples of bacteria include Bacillus bacteria (e.g., B. subtilis, B. megaterium), Acinetobacter bacteria, Norcardia baceteria, Xanthobacter bacteria, Escherichia bacteria (e.g., E. coli (e.g., strains DH10B, Stb12, DH5-alpha, DB3, DB3.1), DB4, DB5, and JDP682, Streptomyces bacteria, Erwinia bacteria, Klebsiella bacteria; Serratia bacteria (e.g., S. marcessans), Pseudomonas bacteria (e.g., P. acruginosa), Salmonella bacteria (e.g., S. typhimurium, S. typhi), Megasphaera bacteria (e.g., Megasphaera elsdenii). Bacteria also include, but are not limited to, photosynthetic bacteria (e.g., green non-sulfur bacteria, Choroflexus bacteria (e.g., C. aurantiacus), Chloronema bacteria (e.g., C. gigateum)), green sulfur bacteria (e.g., Chlorobium bacteria (e.g., C. limicola)), Pelodictyon bacteria (e.g., P. luteolum), purple sulfur bacteria (e.g., Chromatium bacteria (e.g., C. okenii)), and purple non-sulfur bacteria (e.g., Rhodospirillum bacteria (e.g., R. rubrum), Rhodobacter bacteria (e.g., R. Sphaeroides, R. capsulatus), and Rhodomicrobium bacteria (e.g., R. vanellii).

Cells from non-microbial organisms can also be utilized as sources for a heterologous polynucleotides. Examples include insect cells (e.g., Drosophila (e.g., D. melanogaster), Spodoptera (e.g., S. frugiperda Sf9 or Sf21 cells) and Trichoplusa (e.g., High-Five cells); nematode cells (e.g., C. elegans cells); avian cells; amphibian cells (e.g., Xenopus laevis cells); reptilian cells; mammalian cells (e.g., NIH3T3, 293, CHO, COS, VERO, C127, BHK, Per-C6, Bowes melanoma and HeLa cells); and plant cells (e.g., cells from Arabidopsis thaliana, Nicotania tabacum, etc.), including species of the Cannabis genus (e.g., C. satvia, C. indica, and C. ruderalis).

Microorganisms or cells used as parent organisms or sources for heterologous polynucleotides may be isolated from natural sources or are commercially available, for example, from Invitrogen Corporation (Carlsbad, Calif.), American Type Culture Collection (Manassas, Va.), and Agricultural Research Culture Collection (NRRL, Peoria, Ill.).

Host microorganisms and engineered microorganisms of the invention may be provided in any suitable form. For example, such microorganisms may be provided in liquid culture or solid culture (e.g., agar-based medium), which may be a primary culture or may have been passaged (e.g., diluted and cultured) one or more times. Microorganisms also may be provided in frozen form or dry form (e.g., lyophilized). Microorganisms may be provided at any suitable concentration.

### Engineered Pathways

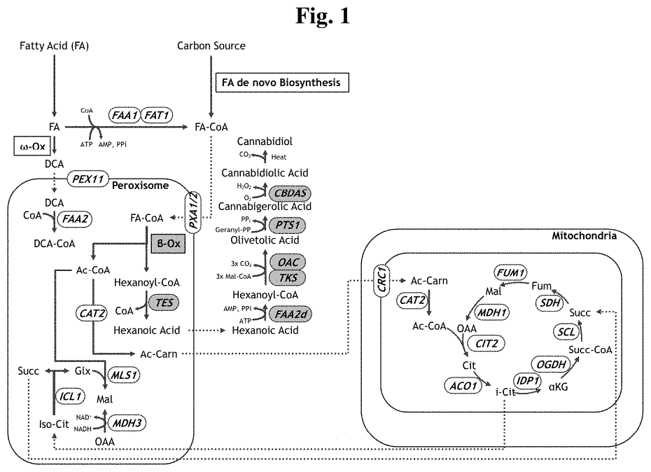

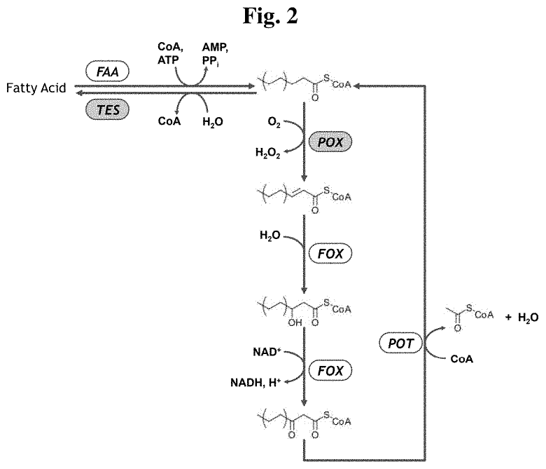

FIGS. 1 and 2 depict embodiments of engineered biosynthetic pathways for making cannabidiol using various feedstocks, including glycerol and fatty acids, or combinations thereof. Indeed, any suitable carbon source (or mixture or combinations of carbon sources), can be used as the feedstock for an engineered microorganism of the invention. In some embodiments, the activities in the pathways depicted in FIGS. 1 and 2 can be engineered to enhance metabolism and CBD formation. In other embodiments, the last enzyme in the CBD biosynthetic pathway (CBDAS) can be substituted for another enzyme to produce a different cannabinoid such as cannabichromenic acid using cannabichromenic acid synthase (CBCAS). In still other embodiments, the last enzyme in the CBD biosynthetic pathway can be omitted from the engineered organism resulting in the production of cannabigerolic acid (CBGA).

In certain embodiments, one or more activities in one or more metabolic pathways can be engineered to increase carbon flux through the engineered pathways to produce CBD. The engineered activities can be chosen to allow increased production of metabolic intermediates that can be utilized in one or more other engineered pathways to achieve increased production of CBD. The engineered activities also can be chosen to allow decreased activity of enzymes that reduce production of a desired intermediate or end product (e.g., reverse activities). As will be appreciated, such carbon flux management can be optimized for any chosen feedstock, by engineering the appropriate activities in the appropriate pathways.

A microorganism may be modified and engineered to include or regulate one or more activities in a fatty acid (e.g., hexanoic acid, octanoic acid, decanoic acid, dodecanoic acid, tetradecanoic acid, hexadecanoic acid, octadecanoic acid, oleic acid, linoleic acid, linolenic acid, eicosanoic acid) pathway. The term “activity” refers to the functioning of a microorganism's natural or engineered biological pathways to yield various desired products, and are generally the result of enzymatic action. A desired enzyme having the desired activity(ies) can be provided by any non-mammalian source in certain embodiments. Such sources include, without limitation, eukaryotes such as yeast and fungi and prokaryotes such as bacteria. In some embodiments, a reverse activity in a pathway described herein can be altered (e.g., disrupted or reduced) to increase carbon flux through the engineered pathway toward production of CBD. In some embodiments, a genetic modification disrupts an activity in an engineered pathway, or disrupts polynucleotide that encodes a polypeptide that catalyzes a particular reaction in the particular pathway.

In some embodiments, a desired activity can be modified to alter the catalytic specificity of a chosen enzyme in a biosynthetic pathway. In some embodiments, the altered catalytic specificity can be found by screening naturally occurring variant or mutant populations of a host organism. In other embodiments, the altered catalytic activity can be generated by various mutagenesis techniques in conjunction with selection and/or screening for the desired activity. In some embodiments, the altered catalytic activity can be generated using a mix and match approach, followed by selection and/or screening for the desired catalytic activity.

An activity within an engineered microorganism provided herein can include one or more (e.g., 1 or more, up to and including all) of the following activities: fatty acyl-CoA oxidase; fatty acyl-CoA synthetase; polyketide synthase (PKS) or tetraketide synthase (TKS); olivetolic acid cyclase (OAC); aromatic prenyltransferase (GOT); geranyl diphosphate synthase (ERG20), farnesyl diphosphate synthase (ERG20), cannabichromenic acid synthase (CBCAS), cannabidiolic acid synthase (CBDAS), and other cannabinoid synthase enzymes acting on CBGA as substrate (e.g., tetrahydrocannabinolic acid synthase). In certain embodiments, one or more of the foregoing activities is altered by way of a genetic modification. In some embodiments, one or more of such activities is altered by way of (i) adding a heterologous polynucleotide that encodes a polypeptide having the activity, and/or (ii) altering or adding a regulatory sequence that regulates the expression of a endogenous polypeptide having the activity. In certain embodiments, one or more of the foregoing activities is altered by way of (i) disrupting an endogenous polynucleotide that encodes a polypeptide having the activity (e.g., insertional mutagenesis), (ii) deleting a regulatory sequence that regulates the expression of a polypeptide having the activity, and/or (iii) deleting the coding sequence that encodes a polypeptide having the activity (e.g., knock out mutagenesis).

### Polynucleotides and Polypeptides

A nucleic acid (e.g., also referred to herein as nucleic acid reagent, target nucleic acid, target nucleotide sequence, nucleic acid sequence of interest or nucleic acid region of interest) can be from any source or composition, such as DNA, cDNA, gDNA (genomic DNA), RNA, siRNA (short inhibitory RNA), RNAi, tRNA or mRNA, for example, and can be in any form (e.g., linear, circular, supercoiled, single-stranded, double-stranded, and the like). A nucleic acid can also comprise DNA or RNA analogs (e.g., containing base analogs, sugar analogs and/or a non-native backbone and the like). It is understood that the term “nucleic acid” does not refer to or infer a specific length of the polynucleotide chain, thus polynucleotides and oligonucleotides are also included in the definition. Deoxyribonucleotides include deoxyadenosine, deoxycytidine, deoxyguanosine and deoxythymidine. For RNA, the uracil base is uridine.

A nucleic acid sometimes is a plasmid, phage, autonomously replicating sequence (ARS), centromere, artificial chromosome, yeast artificial chromosome (e.g., YAC) or other nucleic acid able to replicate or be replicated in a host cell. In certain embodiments a nucleic acid can be from a library or can be obtained from enzymatically digested, sheared, or sonicated genomic DNA (e.g., fragmented) from an organism of interest.

A nucleic acid is sometimes amplified by any suitable amplification process known in the art (e.g., PCR, RT-PCR, and the like). Nucleic acid amplification may be particularly beneficial when using organisms that are typically difficult to culture (e.g., slow growing, require specialize culture conditions and the like).

In some embodiments, a nucleic acid is often stably integrated into the chromosome of the host organism to produce an engineered microorganism, while in other embodiments a nucleic acid can be deleted from of a portion of a host chromosome, in certain embodiments (e.g., genetically modified organisms, where alteration of the host genome confers the ability to selectively or preferentially maintain the desired organism carrying the genetic modification). Such nucleic acids (e.g., nucleic acids or genetically modified organisms whose altered genome confers a selectable trait to the organism) can be selected for their ability to guide production of a desired protein or nucleic acid molecule. When desired, the nucleic acid can be altered such that codons encode for (i) the same amino acid, using a different tRNA than that specified in the native sequence; or (ii) a different amino acid than is normal, including unconventional or unnatural amino acids (including detectably labeled amino acids). As described herein, the term “native sequence” refers to an unmodified nucleotide sequence as found in its natural setting (e.g., a nucleotide sequence as found in a naturally occurring microorganism).

A nucleic acid or nucleic acid reagent can comprise certain elements often selected according to the intended use of the nucleic acid. Any of the following elements can be included in or excluded from a nucleic acid reagent. A nucleic acid reagent, for example, may include one or more or all of the following nucleotide elements: one or more promoter elements; one or more 5′ untranslated regions (5′UTRs); one or more regions into which a target nucleotide sequence may be inserted (an “insertion element”); one or more target nucleotide sequences, one or more 3′ untranslated regions (3′UTRs), and one or more selection elements. A nucleic acid can be provided with one or more of such elements and other elements may be inserted into the nucleic acid before the nucleic acid is introduced into the desired organism. In some embodiments, a provided nucleic acid comprises a promoter, 5′UTR, optional 3′UTR, and insertion element(s) by which a target nucleotide sequence is inserted (i.e., cloned) into the nucleotide acid. In certain embodiments, a provided nucleic acid comprises a promoter, insertion element(s), and optional 3′UTR, and a 5′UTR/target nucleotide sequence is inserted with an optional 3′UTR. The elements can be arranged in any order suitable for expression in the chosen expression system (e.g., expression in a chosen organism, or expression in a cell free system, for example), and in some embodiments a nucleic acid reagent comprises the following elements in the 5′ to 3′ direction: (1) promoter element, 5′UTR, and insertion element(s); (2) promoter element, 5′UTR, and target nucleotide sequence; (3) promoter element, 5′UTR, insertion element(s) and 3′UTR; and (4) promoter element, 5′UTR, target nucleotide sequence and 3′UTR.

### Promoters

A promoter element often comprises a region of DNA that can facilitate transcription of a particular gene by providing a start site for the synthesis of RNA corresponding to the gene. Promoters generally are located near (often upstream of) the gene(s) whose transcription they regulate. In some embodiments, a promoter element can be isolated from a gene and be inserted in functional connection with another polynucleotide sequence to allow altered and/or regulated expression. A non-native promoter (e.g., promoter not normally associated with a given nucleic acid sequence) used for expression of a nucleic acid often is referred to as a heterologous promoter. In certain embodiments, a heterologous promoter and/or a 5′UTR can be inserted in functional connection with a polynucleotide that encodes a polypeptide having a desired activity as described herein. The terms “operably linked” and “in functional connection with” as used herein with respect to promoters, refer to a relationship between a coding sequence and a promoter element. The promoter is operably linked or in functional connection with the coding sequence when expression from the coding sequence via transcription is regulated, or controlled by, the promoter element.

In some embodiments, regulation of a promoter element can be used to alter (e.g., increase, add, decrease or substantially eliminate) the activity of a peptide, polypeptide, or protein (e.g., an enzyme activity). For example, a microorganism can be engineered by genetic modification to express a nucleic acid that can add a novel activity (e.g., an activity not normally found in the host microorganism) or increase (or decrease) the expression of an existing activity by increasing (or decreasing) transcription from a homologous or heterologous promoter operably linked to a nucleotide sequence of interest (e.g., homologous or heterologous nucleotide sequence of interest), in certain embodiments.

### Homology and Identity

In addition to the regulated promoter sequences, regulatory sequences, and coding polynucleotides provided herein, a nucleic acid may include a polynucleotide sequence about 80% or more identical thereto (or to the complementary sequences). That is, a nucleotide sequence that is at least about 80%-100% (inclusive of all percentages, and ranges of percentages, in this range) or more identical to a nucleotide sequence described herein can be utilized. The term “identical” as used herein refers to two or more nucleotide sequences having substantially the same nucleotide sequence when compared to each other. One test for determining whether two nucleotide sequences or amino acids sequences are substantially identical is to determine the percent of identical nucleotide sequences or amino acid sequences shared. Calculations of sequence identity can be performed by any suitable method, including using a mathematical algorithm, e.g., the algorithm of Meyers & Miller, CABIOS 4:11-17 (1989), the Needleman & Wunsch, J. Mol. Biol. 48:444-453 (1970) algorithm incorporated into the GAP program in the GCG software package (available at the “gcg.com” http address on the Worldwide Web). Sequence identity can also be determined by hybridization assays conducted under stringent conditions, i.e., conditions for hybridization and washing. Stringent conditions are known to those skilled in the art and can be found in Current Protocols in Molecular Biology, John Wiley & Sons, N.Y., 6.3.1-6.3.6 (1989).

In the context of amino acid sequence identity as between an amino acid sequence of a polypeptide of interest and that of a reference polypeptide, as described above, the level (expressed preferably as a percentage) of amino acid sequence identity between the amino acid sequences of the reference polypeptide and that of the polypeptide of interest can be determined by any suitable method, and generally ranges from at least about 50% sequence identity to 100% identity. In general, the reference polypeptide and polypeptide of interest share the same enzymatic activity, although in some embodiments the reference polypeptide and/or polypeptide of interest may also have other activities that are not shared. In the context of shared enzymatic activities, the levels of activity may differ as between the reference polypeptide and polypeptide of interest.

UTRs

As noted above, nucleic acid may also comprise one or more 5′ UTR's, and one or more 3′UTR's. A 5′ UTR may comprise one or more elements endogenous to the nucleotide sequence from which it originates, and sometimes includes one or more exogenous elements. A 5′ UTR can originate from any suitable nucleic acid, such as genomic DNA, plasmid DNA, RNA or mRNA, for example, from any suitable organism (e.g., virus, bacterium, yeast, fungi, plant, insect or mammal). Appropriate elements for the 5′ UTR can be selected based on the chosen expression system (e.g., expression in a chosen organism, or expression in a cell free system, for example). A 5′ UTR sometimes comprises one or more of the following elements: enhancer sequences (e.g., transcriptional or translational); a transcription initiation site; a transcription factor binding site; a translation regulation site; a translation initiation site; a translation factor binding site; an accessory protein binding site; a feedback regulation agent binding site; a Pribnow box; a TATA box; a−35 element; an E-box (helix-loop-helix binding element); a ribosome binding site; and internal ribosome entry site (IRES); a silencer element; and the like. In some embodiments, a promoter element may be isolated such that all 5′ UTR elements necessary for proper conditional regulation are contained in the promoter element fragment, or within a functional subsequence of a promoter element fragment.

A 5′ UTR in the nucleic acid can comprise a translational enhancer nucleotide sequence. A translational enhancer nucleotide sequence often is located between the promoter and the target nucleotide sequence in a nucleic acid reagent. A translational enhancer sequence often binds to a ribosome, sometimes is an 18S rRNA-binding ribonucleotide sequence (i.e., a 40S ribosome binding sequence) and sometimes is an internal ribosome entry sequence (IRES). An IRES generally forms an RNA scaffold with precisely placed RNA tertiary structures that contact a 40S ribosomal subunit via a number of specific intermolecular interactions. Examples of ribosomal enhancer sequences are known and/or can be identified by those in the art.

A 3′ UTR can comprise one or more elements endogenous to the nucleotide sequence from which it originates and sometimes includes one or more exogenous elements. A 3′ UTR may originate from any suitable nucleic acid, such as genomic DNA, plasmid DNA, RNA or mRNA, for example, from any suitable organism (e.g., a virus, bacterium, yeast, fungi, plant, insect, or mammal). Appropriate elements for the 3′ UTR can be selected based upon the chosen expression system (e.g., expression in a chosen organism, for example). A 3′ UTR often includes a polyadenosine tail.

In some embodiments, modification of a 5′ UTR and/or a 3′ UTR can be used to alter (e.g., increase, add, decrease, or substantially eliminate) the activity of a promoter. Alteration of the promoter activity can in turn alter the activity of a peptide, polypeptide, or protein (e.g., enzyme activity, for example) by a change in transcription of the nucleotide sequence(s) of interest from an operably linked promoter element comprising the modified 5′ or 3′ UTR. For example, a microorganism can be engineered by genetic modification to express a nucleic acid comprising a modified 5′ or 3′ UTR that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent comprising a modified 5′ or 3′ UTR that can decrease the expression of an activity by decreasing or substantially eliminating transcription from a homologous or heterologous promoter operably linked to a nucleotide sequence of interest, in certain embodiments.

### Target Nucleotide Sequences

A nucleic acid sometimes comprises a target nucleotide sequence. A “target nucleotide sequence” encodes a nucleic acid, peptide, polypeptide, or protein of interest, and may be a ribonucleotide sequence or a deoxyribonucleotide sequence. A target nucleic acid sometimes is an untranslated ribonucleic acid and sometimes is a translated ribonucleic acid. Untranslated ribonucleic acid include small interfering ribonucleic acids (siRNAs), short hairpin ribonucleic acid (shRNAs), other ribonucleic acids capable of RNA interference (RNAi), antisense ribonucleic acids, or ribozymes. A translatable target nucleotide sequence (e.g., a target ribonucleotide sequence) sometimes encodes a peptide, polypeptide, or protein, which are sometimes referred to herein as “target peptides,” “target polypeptides”, or “target proteins”. A target peptide, polypeptide, or protein, or an activity catalyzed thereby, can be encoded by a target nucleotide sequence and can be selected by a user.

### Nucleic Acids and Tools

A nucleic acid sometimes comprises one or more ORFs (open reading frame). An ORF may be from any suitable source, sometimes from genomic DNA, mRNA, reverse-transcribed RNA, or complementary DNA (cDNA) or a nucleic acid library comprising one or more of the foregoing, and can be from any organism species that contains a nucleic acid sequence of interest, protein of interest, or activity of interest. Non-limiting examples of organisms from which an ORF can be obtained include bacteria, yeast, fungi, plant, human, insect, nematode, or mammal, for example.

A nucleic acid sometimes comprises a nucleotide sequence adjacent to an ORF that is translated in conjunction with the ORF and encodes an amino acid tag. The tag-encoding nucleotide sequence can be located 3′ and/or 5′ of an ORF in the nucleic acid, thereby encoding a tag at the C-terminus or N-terminus of the protein or peptide encoded by the ORF. Any tag that does not abrogate in vitro transcription and/or translation can be utilized. Tags may facilitate isolation and/or purification of the desired ORF product from culture or fermentation media. A tag sometimes comprises a sequence that localizes a translated protein or peptide to a component in a system, which may be referred to as a “signal sequence” or “localization signal sequence”. A signal sequence often is incorporated at the N-terminus of a target protein or target peptide, although sometimes it can be incorporated at the C-terminus. Examples of signal sequences are known in the art, can be readily incorporated into an engineered nucleic acid, and often are selected according to the organism in which expression of the nucleic acid is intended. A signal sequence in some embodiments localizes a translated protein or peptide to a cell membrane. Examples of signal sequences include a nucleus targeting signal, a mitochondrial targeting signal, a peroxisome targeting signal (e.g., C-terminal sequence SKL), and a secretion.

A tag sometimes is directly adjacent to the amino acid sequence encoded by an ORF (i.e., there is no intervening sequence) and sometimes a tag is substantially adjacent to an ORF encoded amino acid sequence (e.g., an intervening sequence is present). An intervening sequence sometimes is referred to herein as a “linker sequence,” and may be of any suitable length. A linker can be of any suitable amino acid length and content, and often comprises a higher proportion of amino acids having relatively short side chains (e.g., glycine, alanine, serine, and threonine). A linker sequence sometimes encodes an amino acid sequence that is about 1 to about 20 amino acids in length, and sometimes about 5 to about 10 amino acids in length.

Any convenient cloning strategy can be utilized to incorporate an element such as an ORF, a promoter, etc. into a target nucleic acid to produce an engineered nucleic acid. Known methods can be utilized to insert an element into the template independent of an insertion element, such as (1) cleaving the template, at one or more existing restriction enzyme sites and ligating an element of interest and (2) adding restriction enzyme sites to the template by hybridizing oligonucleotide primers that include one or more suitable restriction enzyme sites and amplifying by polymerase chain reaction (described in greater detail herein). Other cloning strategies take advantage of one or more insertion sites present or inserted into the nucleic acid reagent, such as an oligonucleotide primer hybridization site for PCR, for example, and others described herein. In some embodiments, a cloning strategy can be combined with genetic manipulation such as recombination (e.g., recombination of a nucleic acid reagent with a nucleic acid sequence of interest into the genome of the organism to be modified, as described further herein).

In some embodiments, the nucleic acid includes one or more recombinase insertion sites. A recombinase insertion site is a recognition sequence on a nucleic acid molecule that participates in an integration/recombination reaction by recombination proteins. Examples of recombinase cloning nucleic acids are in GATEWAY® systems (Invitrogen, California), which include at least one recombination site for cloning a desired nucleic acid molecules in vivo or in vitro. A representative recombination system useful for engineering yeast makes use of the URA3 gene (e.g., for S. cerevisiae and C. albicans, for example) or URA4 and URA5 genes (e.g., for S. pombe, for example) and toxicity of the nucleotide analogue 5-Fluoroorotic acid (5-FOA). The URA3 or URA4 and URA5 genes encode orotidine-5′-monophosphate (OMP) decarboxylase. Yeast with an active URA3 or URA4 and URA5 gene (phenotypically Ura+) convert 5-FOA to fluorodeoxyuridine, which is toxic to yeast cells. Yeast carrying a mutation in the appropriate gene(s) or having a knock out of the appropriate gene(s) can grow in the presence of 5-FOA, if the media is also supplemented with uracil.

A plasmid or expression vector can be made which may comprise the URA3 gene or cassette (for S. cerevisiae, for example) flanked on either side by the same nucleotide sequence in the same orientation. The URA3 expression cassette comprises a promoter, a URA3 gene, and a functional transcription terminator. Targeting sequences, which direct the construct to a particular nucleic acid region of interest in the microorganism to be engineered, are added such that the targeting sequences are adjacent to and abut the flanking sequences on either side of the URA3 cassette. Yeast can be transformed with the engineering construct and plated on minimal media without uracil. Colonies can be screened by PCR to determine those transformants that have the expression cassette inserted in the proper location in the genome. Checking insertion location prior to selecting for recombination of the ura3 cassette may reduce the number of incorrect clones carried through to later stages of the procedure. Correctly inserted transformants can then be replica-plated on minimal media containing 5-FOA to select for recombination of the URA3 cassette out of the construct, leaving a disrupted gene and an identifiable footprint (e.g., nucleic acid sequence) that can be used to verify the presence of the disrupted gene. The technique described is useful for disrupting or “knocking out” gene function, but also can be used to insert genes or constructs into a host microorganism genomes in a targeted, sequence-specific manner.

In some embodiments, other auxotrophic or dominant selection markers can be used in place of URA3 (e.g., an auxotrophic selectable marker), with the appropriate change in selection media and selection agents. Auxotrophic selectable markers are used in strains deficient for synthesis of a required biological molecule (e.g., amino acid or nucleoside, for example). Non-limiting examples of additional auxotrophic markers include HIS3, TRP-1, LEU2, LEU2-d, and LYS2. Certain auxotrophic markers (e.g., URA3 and LYS2) allow counter selection to select for a second recombination event.

Dominant selectable markers are useful because they also allow industrial and/or prototrophic strains to be used for genetic manipulations. Additionally, dominant selectable markers provide the advantage that rich medium can be used for plating and culture growth, and thus growth rates are markedly increased.

In other embodiments, a nucleic acid includes one or more topoisomerase insertion sites. A topoisomerase insertion site is a defined nucleotide sequence recognized and bound by a site-specific topoisomerase. For example, the nucleotide sequence 5′-(C/T)CCTT-3′ is a topoisomerase recognition site bound specifically by most pox virus topoisomerases, including vaccinia virus DNA topoisomerase I. After binding to the recognition sequence, the topoisomerase cleaves the strand at the 3′-most thymidine of the recognition site to produce a nucleotide sequence comprising 5′-(C/T)CCTT-PO4-TOPO, a complex of the topoisomerase covalently bound to the 3′ phosphate via a tyrosine in the topoisomerase (e.g., Shuman, J. Biol. Chem. 266:11372-11379, 1991; Sekiguchi and Shuman, Nucl. Acids Res. 22:5360-5365, 1994; U.S. Pat. No. 5,766,891; PCT/US95/16099; and PCT/US98/12372). In comparison, the nucleotide sequence 5′-GCAACTT-3′ is a topoisomerase recognition site for type IA E. coli topoisomerase III. An element to be inserted often is combined with topoisomerase-reacted template and thereby incorporated into the nucleic acid reagent (e.g., TOPO TA Cloning® Kit and Zero Blunt® TOPO®, Thermo Fisher Scientific).

A plasmid or expression vector often contains one or more origin of replication (ORI) elements. In some embodiments, a plasmid or expression vector comprises two or more ORIs, where one functions efficiently in one organism (e.g., a bacterium) and another functions efficiently in another organism (e.g., a eukaryote such as yeast, for example). In some embodiments, an ORI may function efficiently in one species (e.g., S. cerevisiae, for example) and another ORF may function efficiently in a different species (e.g., S. pombe, for example). A plasmid or expression vector often also includes one or more transcription regulation sites (e.g., promoters, transcription termination signals, polyadenylation sites, etc.).

A plasmid or expression vector also typically includes one or more selection elements (e.g., elements for selection of the presence of the plasmid or expression vector). Selection elements often are utilized using known processes to determine whether a plasmid or expression vector is included in a recombinant cell. In some embodiments, a plasmid or expression vector includes two or more selection elements, where one functions efficiently in one microorganism (e.g., a bacterium) and another functions efficiently in another microorganism (e.g., a eukaryote such as a yeast). Examples of selection elements include, but are not limited to, (1) nucleic acids that encode products that provide resistance against otherwise toxic compounds (e.g., antibiotics), (2) nucleic acid that encode products that are otherwise lacking in the recipient cell (e.g., essential products, tRNA genes, auxotrophic markers, etc.), (3) nucleic acids that encode products that suppress the activity of a gene product, (4) nucleic acids that encode products that can be readily identified (e.g., phenotypic markers such as antibiotics (e.g., beta-lactamase), beta-galactosidase, green fluorescent protein (GFP), yellow fluorescent protein (YFP), red fluorescent protein (RFP), cyan fluorescent protein (CFP), and cell surface proteins), (5) nucleic acids that bind products that are otherwise detrimental to-cell survival and/or function, (6) nucleic acids that otherwise inhibit the activity of any of the nucleic acids described in nos. 1-5, above (e.g., antisense oligonucleotides), (7) nucleic acids that bind products that modify a substrate (e.g., restriction endonucleases), (8) nucleic acids that can be used to isolate or identify a desired molecule (e.g., specific protein binding sites), (9) nucleic acids that encode a specific nucleotide sequence that can be otherwise non-functional (e.g., for PCR amplification of subpopulations of molecules), (10) nucleic acids that, when absent, directly or indirectly confer resistance or sensitivity to particular compounds, (11) nucleic acids that encode products that either are toxic or convert a relatively non-toxic compound to a toxic compound (e.g., Herpes simplex thymidine kinase, cytosine deaminase, etc.) in recipient cells, (12) nucleic acids that inhibit replication, partition, or heritability of nucleic acid molecules that contain them, and/or (13) nucleic acids that encode conditional replication functions, e.g., replication in certain hosts or host cell strains or under certain environmental conditions (e.g., temperature, nutritional conditions, and the like).

A nucleic acid is of any form useful for in vivo transcription and/or translation. A nucleic acid sometimes is a plasmid, such as a supercoiled plasmid, sometimes is a yeast artificial chromosome (e.g., YAC), sometimes is a linear nucleic acid (e.g., a linear nucleic acid produced by PCR or by restriction digest), sometimes is single-stranded and sometimes is double-stranded. A nucleic acid reagent sometimes is prepared by an amplification process, such as a polymerase chain reaction (PCR) process or transcription-mediated amplification process (TMA). Standard PCR processes are known and generally are performed in cycles that heat denaturation of double-stranded templates; cooling, in which primer oligonucleotides hybridize to targeted primer binding sites; and extension of the oligonucleotides by a polymerase (e.g., Taq polymerase). Multiple cycles frequently are performed using a commercially available thermal cycler.

In some embodiments, a nucleic acid, protein, protein fragment, or other reagent described herein is isolated or purified. Here, “isolated” refers to material removed from its original environment (e.g., the natural environment if it is naturally occurring, or a host cell if expressed exogenously), and thus is altered “by the hand of man” from its original environment, while “purified”, with reference to a particular molecule, does not refer to absolute purity. Sometimes, a protein or nucleic acid is “substantially pure,” indicating that the protein of nucleic acid represents at least 50% of protein or nucleic acid on a mass basis of the composition. Often, a substantially pure protein, nucleic acid, or other specified compound is at least about 75% on a mass basis of the composition, and sometimes at least about 95% on a mass basis of the composition.

### Engineering and Alteration Methods

The methods and compositions (e.g., nucleic acids) described herein can be used to generate engineered microorganisms. As described elsewhere herein, the term “engineered microorganism” refers to a modified microorganism that includes one or more activities distinct from an activity present in a microorganism utilized as a starting point for modification (e.g., host microorganism or unmodified organism). Engineered microorganisms typically arise as a result of a genetic modification, usually introduced or selected for, by one of skill in the art using readily available techniques. Non-limiting examples of methods useful for generating an altered activity include, introducing a heterologous polynucleotide (e.g., nucleic acid or gene integration, also referred to as “knock in”), removing an endogenous polynucleotide, altering the sequence of an existing endogenous nucleic acid sequence (e.g., site-directed mutagenesis), disruption of an existing endogenous nucleic acid sequence (e.g., knock-outs and transposon or insertion element mediated mutagenesis), selection for an altered activity where the selection causes a change in a naturally occurring activity that can be stably inherited (e.g., causes a change in a nucleic acid sequence in the genome of the organism or in an epigenetic nucleic acid that is replicated and passed on to daughter cells), PCR-based mutagenesis, and the like. The term “mutagenesis” refers to any modification to a nucleic acid (e.g., nucleic acid or host chromosome, for example) that is subsequently used to generate a product in a host or modified microorganism. Non-limiting examples of mutagenesis include deletion, insertion, substitution, rearrangement, point mutations, suppressor mutations, and the like. Mutagenesis methods are known in the art. Non-limiting examples can also be found in Maniatis, T., E. F. Fritsch and J. Sambrook (1982) Molecular Cloning: a Laboratory Manual; Cold Spring Harbor Laboratory, Cold Spring Harbor, N.Y.

In some embodiments, a microorganism engineered using the methods and nucleic acids described herein can produce cannabinoids. In certain embodiments, the engineered microorganism that produces cannabinoids may comprise one or more altered activities. In some embodiments, an engineered microorganism as described herein may comprise a genetic modification that adds or increases the activities needed to biosynthetically produce cannabinoids in such microorganism.

An added activity often is an activity not detectable in a host microorganism absent engineering, although added activity is also understood to include increased activity of an endogenous (or previously added) activity. An increased activity generally is an activity detectable after introduction into the parent microorganism of an engineered construct or pathway (or multiple engineered pathways). An activity can be increased to any suitable level for production of the desired product, for example, CBD, including an increase less than 2-fold (e.g., about 1% increase to about 100% increase, inclusive), 2-fold to 1,000-fold or more increase (inclusive of any increase, or range of increases, within the stated range). An activity may be added or increased by any suitable approach, including by increasing the copy number of a polynucleotide that encodes a polypeptide having a desired activity. In some embodiments, copy number is increased by 1 to about 100 additional copies (inclusive of any increase, or range of increases, within the stated range). In certain embodiments an activity can be added or increased by inserting into a host microorganism a polynucleotide that encodes a heterologous polypeptide (or plurality of heterologous polypeptides) having the added activity(ies) or a modified endogenous polypeptide or plurality of endogenous polypeptides). A “modified endogenous polypeptide” often has an activity different than an activity of a native polypeptide counterpart (e.g., different catalytic activity and/or different substrate specificity), and often is active (e.g., an activity (e.g., substrate turnover) is detectable). In certain embodiments, an activity can be added or increased by inserting into a host microorganism a heterologous polynucleotide that is (i) operably linked to another polynucleotide that encodes a polypeptide having the added activity, and (ii) up regulates production of the polynucleotide. Thus, an activity can be added or increased by inserting or modifying a regulatory polynucleotide operably linked to another polynucleotide that encodes a polypeptide having the target activity. In certain embodiments, an activity can be added or increased by subjecting a host microorganism to a selective environment and screening for microorganisms that have a detectable level of the target activity.

Similarly, a reduced or inhibited activity generally is an activity detectable in a host microorganism that has been reduced or inhibited in an engineered microorganism. An activity can be reduced to undetectable levels in some embodiments, or detectable levels in certain embodiments. An activity can be decreased to any suitable level for production, including a reduction of less than 2-fold (e.g., about 1% decrease to about 100% decrease, inclusive), 2-fold to 1,000-fold or more (up to and including a decrease to undetectable levels) decrease (inclusive of any decrease, or range of decreases, within the stated range). An activity may be reduced or removed by decreasing the number of copies of a polynucleotide that encodes a polypeptide having a target activity, in some embodiments. In some embodiments, an activity can be reduced or removed by (i) inserting a polynucleotide within a polynucleotide that encodes a polypeptide having the target activity (disruptive insertion), and/or (ii) removing a portion of or all of a polynucleotide that encodes a polypeptide having the target activity (deletion or knockout, respectively). In certain embodiments, an activity can be reduced or removed by inserting into a host microorganism a heterologous polynucleotide that is (i) operably linked to another polynucleotide that encodes a polypeptide having the target activity, and (ii) down regulates production of the polynucleotide. Thus, in some embodiments an activity can be reduced or removed by inserting or modifying a regulatory polynucleotide operably linked to another polynucleotide that encodes a polypeptide having the target activity. Like, in some embodiments, an untranslated ribonucleic acid, or a cDNA can be used to reduce the expression of a particular activity or enzyme.

In certain embodiments, nucleotide sequences sometimes are added to, modified or removed from one or more of the nucleic acid elements, such as the promoter, 5′UTR, target sequence, or 3′UTR elements, to enhance, potentially enhance, reduce, or potentially reduce transcription and/or translation before or after such elements are incorporated in a nucleic acid. Such elements include AU-rich elements (AREs, e.g., AUUUA repeats) and/or splicing junctions that follow a non-sense codon that may be removed from or modified in a 3′UTR. A polyadenylation signal may also be included in or removed from a 3′UTR.

In some embodiments, an activity can be altered by modifying the nucleotide sequence of an ORF. An ORF sometimes is mutated or modified (for example, by point mutation, deletion mutation, insertion mutation, PCR-based mutagenesis, and the like) to alter, enhance or increase, reduce, substantially reduce, or eliminate the activity of the encoded protein or peptide. The protein or peptide encoded by a modified ORF sometimes is produced in a lower amount or may not be produced at detectable levels, and in other embodiments, the product or protein encoded by the modified ORF is produced at a higher level (e.g., codons sometimes are modified so they are compatible with tRNA's preferentially used in the host organism or engineered organism). To determine the relative activity, the activity from the product of the mutated ORF (or cell containing it) can be compared to the activity of the product or protein encoded by the unmodified ORF (or cell containing it).

In some embodiments, an ORF nucleotide sequence sometimes is mutated or modified to alter the triplet nucleotide sequences used to encode amino acids (e.g., amino acid codon triplets, for example). Modification of the nucleotide sequence of an ORF to alter codon triplets sometimes is used to change the codon found in the original sequence to better match the preferred codon usage of the organism in which the ORF or nucleic acid will be expressed. For example, as is known, the codon usage and therefore the codon triplets encoded by a nucleic acid sequence in bacteria may be different from the preferred codon usage in eukaryotes, like yeast or plants. Preferred codon usage also may be different between bacterial species. In certain embodiments an ORF nucleotide sequences sometimes is modified to eliminate codon pairs and/or eliminate mRNA secondary structures that can cause pauses during mRNA translation. Translational pausing sometimes occurs when nucleic acid secondary structures exist in an mRNA, and sometimes occurs due to the presence of codon pairs that slow the rate of translation by causing ribosomes to pause. In some embodiments, the use of lower abundance codon triplets can reduce translational pausing due to a decrease in the pause time needed to load a charged tRNA into the ribosome translation machinery. Therefore, to increase transcriptional and translational efficiency in bacteria (e.g., where transcription and translation are much more compartmentally and temporally linked, for example) or to increase translational efficiency in eukaryotes (e.g., where transcription and translation are functionally separated), the nucleotide sequence of a nucleotide sequence of interest can be altered to better suit the transcription and/or translational machinery of the host and/or genetically modified microorganism.

Codons can be altered and optimized (i.e., codon optimization) according to the preferred usage by a given organism by determining the codon distribution of the nucleotide sequence donor organism and comparing the distribution of codons to the distribution of codons in the recipient or host microorganism. Techniques known in the art (e.g., site directed mutagenesis and the like) can then be used to alter the codons accordingly. Comparisons of codon usage can be done by hand or using commercially available nucleic acid analytical software.

Modification of the nucleotide sequence of an ORF also can be used to correct codon triplet sequences that have diverged in different organisms. For example, certain yeast (e.g., C. tropicalis and C. maltose) use the amino acid triplet CUG (e.g., CTG in the DNA sequence) to encode serine. CUG typically encodes leucine in most organisms. In order to maintain the correct amino acid in the resultant polypeptide or protein, the CUG codon must be altered to reflect the engineered microorganism in which the nucleic acid will be expressed. Thus, if an ORF from a bacterial donor is to be expressed in either Candida yeast strain mentioned above, the heterologous nucleotide sequence must first be altered or modified to the appropriate leucine codon. Therefore, in some embodiments, the nucleotide sequence of an ORF sometimes is altered or modified to correct for differences that have occurred in the evolution of the amino acid codon triplets between different organisms. In some embodiments, the nucleotide sequence can be left unchanged at a particular amino acid codon, if the amino acid encoded is a conservative or neutral change in amino acid when compared to the originally encoded amino acid.

In some embodiments, an activity can be altered by modifying translational regulation signals such as stop codons, for example. A stop codon at the end of an ORF can sometimes be modified to another stop codon, such as an amber stop codon. In some embodiments, a stop codon is introduced within an ORF, sometimes by insertion or mutation of an existing codon. An ORF comprising a modified terminal stop codon and/or internal stop codon often is translated in a system comprising a suppressor tRNA that recognizes the stop codon, and in some cases the system is engineered so that a suppressor tRNA charged with an unnatural amino acid results in that amino acid being incorporated during protein synthesis. Methods for incorporating unnatural amino acids into a desired protein or peptide are known.

Depending on the portion of a nucleic acid (e.g., promoter, 5′ or 3′UTR, ORF, and the like) chosen for alteration (e.g., by mutagenesis, for example), the resulting modification(s) can alter a given activity by (i) increasing or decreasing feedback inhibition mechanisms, (ii) increasing or decreasing promoter initiation, (iii) increasing or decreasing translation initiation, (iv) increasing or decreasing translational efficiency, (v) modifying localization of peptides or products expressed from nucleic acid reagents described herein, or (vi) increasing or decreasing the copy number of a nucleotide sequence of interest, and (vii) expression of an anti-sense RNA, RNAi, siRNA, ribozymes, and the like.

In certain embodiments, alteration of a nucleotide sequence can alter sequences involved in transcription initiation (e.g., promoters, 5′ UTR, and the like). A modification sometimes can be made that can enhance or increase initiation from an endogenous or heterologous promoter element. Similarly, a modification sometimes can be made that removes or disrupts sequences that increase or enhance transcription initiation, resulting in a decrease or elimination of transcription from an endogenous or heterologous promoter element.

In some embodiments, alteration of a nucleotide sequence can alter sequences involved in translational initiation or translational efficiency (e.g., 5′ UTR, 3′ UTR, codon triplets of higher or lower abundance, translational terminator sequences and the like, for example). A modification sometimes can be made that can increase or decrease translational initiation, modifying a ribosome binding site for example. A modification sometimes can be made that can increase or decrease translation efficiency.

In certain embodiments, alteration of a nucleotide sequence can alter sequences involved in the localization of peptides, proteins, or other desired products (e.g., CBD, for example). A modification sometimes can be made that can alter (e.g., add or remove) sequences responsible for targeting a polypeptide, protein, or product to an intracellular organelle, the periplasm, cellular membrane, or extracellularly. Transport of a heterologous product to a different intracellular space or extracellularly sometimes can reduce or eliminate toxicity, etc.

In some embodiments, alteration of a nucleotide sequence can alter sequences involved in increasing or decreasing the copy number of a nucleotide sequence of interest. A modification sometimes can be made that increases or decreases the number of copies of an ORF stably integrated into the genome of an engineered microorganism. Non-limiting examples of alterations that can increase the number of copies of a sequence of interest include adding copies of the sequence of interest by duplication of regions in the genome (e.g., adding additional copies by recombination or by causing gene amplification of the host genome, for example), cloning additional copies of a sequence onto an extrachromosomal element (e.g., a plasmid, YAC, etc.), or altering an ORI to increase the number of copies of a plasmid, for example. Non-limiting examples of alterations that can decrease the number of copies of a sequence of interest include removing copies of such sequence by deletion or disruption of regions in the genome, removing additional copies of the sequence from plasmids or other stably maintained, segregating extrachromosomal elements, or altering an ORI to decrease the copy number.

In certain embodiments, increasing or decreasing the expression of a nucleotide sequence of interest can also be accomplished by altering, adding, or removing sequences involved in the expression of an anti-sense RNA, RNAi, siRNA, ribozyme and the like.

Nucleic acid sequences encoding a desired activity can be isolated from cells of a suitable organism using nucleic acid purification procedures known in the art (e.g., Maniatis, et al. (1982), supra) or using commercially available and DNA purification reagents and kits.

In some embodiments, nucleic acid sequences prepared by isolation or amplification can be used, without any further modification, to add an activity to a microorganism and thereby create a genetically modified or engineered microorganism. In certain embodiments, nucleic acid sequences prepared by isolation or amplification can be genetically modified to alter (e.g., increase or decrease, for example) a desired activity. In some embodiments, nucleic acids, used to add an activity to an organism, sometimes are genetically modified to optimize the heterologous polynucleotide sequence encoding the desired activity (e.g., polypeptide or protein, for example). The term “optimize” as used herein can refer to alteration to increase or enhance expression by preferred codon usage. The term optimize can also refer to modifications to the amino acid sequence to increase the activity of a polypeptide or protein, such that the activity exhibits a higher catalytic activity as compared to the “natural” (i.e., unmodified) version of the polypeptide or protein.

Nucleic acid sequences of interest can be genetically modified using methods known in the art.

Mutagenesis techniques are particularly useful for making genetic modification(s). Mutagenesis allows one to alter the genetic information of an organism in a stable manner, either naturally (e.g., isolation using selection and screening) or experimentally by the use of chemicals, radiation, or inaccurate DNA replication (e.g., PCR mutagenesis). In some embodiments, genetic modification can be performed by whole-scale synthetic synthesis of nucleic acids using a native nucleotide sequence as the reference sequence and modifying nucleotides that can result in the desired alteration of activity. Mutagenesis methods sometimes are specific or targeted to specific regions or nucleotides (e.g., site-directed mutagenesis, PCR-based site-directed mutagenesis, and in vitro mutagenesis techniques such as transplacement and in vivo oligonucleotide site-directed mutagenesis, for example). Mutagenesis methods sometimes are non-specific or random with respect to the placement of genetic modifications (e.g., chemical mutagenesis, insertion element (e.g., insertion or transposon elements) and inaccurate PCR-based methods, for example).

In contrast to site-directed or specific mutagenesis, random mutagenesis does not require any sequence information and can be accomplished by a number of widely different methods. Random mutagenesis often is used to create mutant libraries that can be used to screen for the desired genotype or phenotype. Non-limiting examples of random mutagenesis include chemical mutagenesis, UV-induced mutagenesis, insertion element or transposon-mediated mutagenesis, DNA shuffling, error-prone PCR mutagenesis, and the like.

A native, heterologous, or mutagenized polynucleotide can be introduced into a target nucleic acid for introduction into a host organism to create an engineered microorganism. Standard recombinant DNA techniques (restriction enzyme digests, ligation, and the like) can be used to combine a mutagenized nucleic acid of interest into a suitable nucleic acid capable of (i) being stably maintained by selection in the host organism, or (ii) being integrated into the genome of the host organism. As noted above, sometimes nucleic acids comprise two replication origins to allow the same nucleic acid to be manipulated in bacteria before final introduction of the final product into a host microorganism (e.g., yeast or fungus, for example). Standard molecular biology and recombinant DNA methods are known (e.g., described in Maniatis, et al. (1982), supra).

Nucleic acids can be introduced into microorganisms using various techniques, including transformation, transaction, transduction, electroporation, ultrasound-mediated transformation, particle bombardment, and the like. In some instances the addition of carrier molecules (e.g., bis-benzimdazolyl compounds can increase the uptake of DNA in cells typically thought to be difficult to transform by conventional methods.

### Enzymatic Steps to Produce Cannabinoids

Production of hexanoyl-CoA

The hexanoyl-CoA at the beginning of the cannabinoid biosynthetic pathway can be produced by multiple possible routes native to C. sativa, including degradation of polyunsaturated fatty acids by the action of lipoxygenase or de novo fatty acid synthesis. In the lipoxygenase route, the hexanoyl-CoA is thought to be provided by degradation of the unsaturated acid, oleic acid (C18:1), by desaturation to linoleic acid (C18:2) followed by hydroperoxidation by lipoxygenase and finally cleavage to a 6-carbon and 12-carbon molecule by hydroperoxide lyase. The 6-carbon molecule hexanal is then converted to hexanoyl-CoA through multiple enzymatic steps (Hatanaka, 1999; Marks, et al., 2009). In the fatty acid synthesis route, hexanoate is produced by fatty acid synthase with a 6-carbon specific thioesterase to prematurely stop fatty acid synthesis (normally fatty acid synthesis would continue to 16- or 18-carbon fatty acids). The free hexanoate produced by fatty acid synthesis can then be converted to hexanoyl-CoA by an acyl-CoA synthase enzyme. The acyl-activating enzyme from C. sativa with activity on hexanoate is named AAE1 (Stout, et al., 2012).

### Production of Olivetolic Acid

Production of olivetolic acid requires two enzymes, a polyketide synthase (PKS) and olivetolic acid cyclase (OAC). A preferred polyketide synthase is a type III PKS that produces olivetol in vitro (Taura, et al., 2009). Olivetolic acid cyclase (OAC) is the companion enzyme to PKS in producing olivetolic acid. It was confirmed that the PKS on its own produces olivetol, whereas in concert with OAC it produces olivetolic acid (Gagne, et al., 2012). The enzymes do not appear to require direct interaction, but rather the substrate diffuses between the enzymes. The PKS and OAC have been co-expressed in yeast and shown to produce olivetolic acid when the culture was provided with sodium hexanoate.

### Production of Cannabigerolic Acid

The production of cannabigerolic acid results from a condensation step catalyzed by geranylpyrophosphate:olivetolate geranyltransferase (GOT or PT1 or CBGAS). The enzyme was characterized using C. sativa extracts showing it was specific for olivetolic acid and did not accept olivetol as a substrate (Fellermeier and Zenk, 1998). Page and Boubakir in U.S. Pat. No. 8,884,100 (2014) describe the isolation of a gene from C. sativa using the EST sequence identified as CAN121 in Marks, et al. (2009). This gene was used to express the protein in insect cells and yeast. In vitro tests showed geranyltransferase activity, although it is understood that the sample preparation used was a microsomal fraction, whereas the fraction reportedly used in the Fellermeier and Zenk work (1998; see above) was done with soluble protein. While this enzyme is reported as an integral membrane protein (Zirpel, et al., 2017), the referenced works do not indicate anything other than the enzyme is associated with the microsomal fraction. To avoid working with the reportedly transmembrane protein PT1, the soluble enzyme NphB from Streptomyces sp. strain CL190 can instead be used, as this enzyme only accepts geranylpyrophosphate as prenyl donor but is promiscuous as to the aromatic acceptor molecule.

### Production of Cannabidiolic Acid

The last step converting cannabigerolic acid to cannabidiolic acid is performed in an oxidocyclization reaction by cannabidiolic acid synthase (CBDAS) (Taura, et al., 1996). The gene for the enzyme has been cloned from C. sativa, allowing further characterization of the enzyme and demonstration of structural and functional similarity with tetrahydrocannabinolic acid synthase (THCAS) (Taura, et al., 2007; Sirikantaramas, et al., 2004). The enzyme contains a covalently bound FAD involved in the oxidation of substrate. Molecular oxygen serves as electron acceptor from FADH₂, forming hydrogen peroxide and FAD. The enzyme has a signal peptide at its N-terminus for targeting via the secretory pathway to the storage cavity of glandular trichomes in C. sativa (Sirikantaramas, et al., 2005). The synthesis of CBDA and THCA outside of the plant cell in the storage cavity may be due to toxicity of cannabigerolic acid as well as the product cannabinoids.

### Production of Other Cannabinoids

The enzyme acting on cannabigerolic acid as its substrate can be alternately substituted in engineered microorganisms to produce cannabinoids with chemical structures distinct from cannabidiolic acid. For instance, cannabichromenic acid synthase (CBCAS, Morimoto, et al., 1998) can be substituted for CBDAS to result in an engineered organism producing cannabichromenic acid (CBCA). Similarly, tetrahydrocannabinolic acid synthase (THCAS) can be substituted for CBDAS to result in an engineered organism producing THCA. Production of a range of other cannabinoids can be achieved by alternately substituting enzyme activities downstream of the intermediate substrate CBGA.

### Strategy for Pathway Engineering in Yeast

An overview of a representative engineered biosynthetic CBD production pathway according to the invention is shown in FIG. 1. Enzymatic steps or pathways that are targets for engineering or heterologous introduction are highlighted in gray.

Fatty acids transported into the cell can be activated to CoA thioesters by cytosolic acyl-CoA synthetases. Long chain fatty acids are preferably in the activated form to facilitate their transport into the peroxisome. The native pathway for de novo fatty acid biosynthesis also produces long chain fatty acyl-CoA, typically of 16 or 18 carbons in length. In certain preferred yeast embodiments, free fatty acids can also be converted to dicarboxylic acids by the omega-oxidation pathway. Dicarboxylic acids do not need to be activated to CoA thioesters for transport into the peroxisome, and instead they can become substrates for beta-oxidation after activation by peroxisomal acyl-CoA synthetases. Once long chain fatty acyl-CoA substrates are inside the peroxisome they can be shortened two carbons at a time by, for example, the cyclic beta-oxidation pathway. Even-numbered long chain fatty acyl-CoA substrates are typically completely converted to acetyl-CoA using this pathway. Peroxisomal acetyl-CoA is converted by the enzyme carnitine acetyl transferase to acetyl carnitine for transport into mitochondria, where energy is produced for the cell through the action of the TCA cycle and oxidative phosphorylation.

The production of hexanoyl-CoA to initiate the cannabinoid pathway has long been recognized as a challenge (Carvalho, et al., 2017), with the best microbial production of hexanoic acid from K. marxianus at 154 mg/L (Cheon, et al., 2014). This required the introduction of 5 to 7 heterologous genes for the conversion of glycolysis-derived acetyl-CoA to hexanoate. In the instant invention, fatty acids are instead used as feedstock and an engineered beta-oxidation pathway to generate hexanoate is used to initiate the cannabinoid pathway.

A preferred production host, C. viswanathii, has a peroxisomal beta-oxidation pathway that is shown in FIG. 2. The first enzyme of peroxisomal beta-oxidation is any acyl-CoA oxidase, which in certain preferred embodiments is encoded by the genes PDX4 and PDX5 (a nonfunctional PDX2 may also be present). The enzymes encoded by the two genes have different substrate specificities, with Pox4p exhibiting broad chain length specificity and Pox5p exhibiting a lower activity on substrates less than 10 carbons in length (Picataggio, et al., 1991). Since the acyl-CoA oxidase enzyme controls the entry of a substrate into a round of chain shortening, enzymes with differing substrate specificities can be used to produce diacids or fatty acids that would not normally be produced (Picataggio and Beardslee, 2016). For instance, a mutant or heterologous enzyme with no activity on C6 substrates introduced into a Pox4⁻, Pox5⁻ background can be used to produce hexanoyl-CoA (or the 6-carbon diacid adipoyl-CoA). Engineered strains of this type have been called “partially blocked in beta-oxidation” because they do not fully oxidize substrates as in wild-type strains. As the hexanoyl-CoA is not a substrate for beta-oxidation, the CoA is removed by peroxisomal thioesterases to recycle the CoA. The free hexanoate can then diffuse out of the peroxisome into the cytoplasm.

In the instant invention, a number of partially beta-blocked strains that produce hexanoate have been engineered. In particular, engineered strains expressing the heterologous acyl-CoA oxidase from Arthrobacter ureafaciens (AuACO) in a Pox4⁻, Pox5⁻ background produce hexanoate. The AuACO enzyme does not accept diacids as a substrate and evidently has low activity on hexanoyl-CoA. These engineered strains have produced hexanoate at greater than 1 g/L.

In some embodiments, it may be possible to use mutants of the Pox4p or Pox5p enzymes from C. viswanathii to produce hexanoate.

Another option for production of hexanoyl-CoA may be to use the hexanoate synthase (HexS) enzyme from Aspergillus parasiticus. The HexS multisubunit enzyme normally works in concert with a polyketide synthase to produce aflatoxin in Aspergillus species.

As is known, hexanoyl-CoA produced in the peroxisome cannot diffuse into the cytoplasm due to the large coenzyme A group. Thioesterase enzymes that are native to the production strain can carry out the release of the coenzyme A in the peroxisome. For example, the C. viswanathii genome encodes eight thioesterase isozymes that are all targeted to the peroxisome. These thioesterases have different substrate specificities. In some embodiments, it may be desirable to amplify the expression of a peroxisomal thioesterase with activity on 6-carbon substrates to accelerate hexanoate production.

For subsequent use in an engineered cannabinoid biosynthetic production pathway, cytoplasmic hexanoate must be activated back to the CoA thioester form. In certain preferred embodiments, the heterologous acyl-activating enzyme from C. sativa (CsAAE1) may be employed for this reaction. There are, however, peroxisomal enzymes native to C. viswanathii that could also be used. For example, C. viswanathii encodes four peroxisomal acyl-CoA synthetase isozymes (FAA2a-FAA2d). The substrate specificity for these enzymes has been investigated, and the enzyme encoded by FAA2d has been determined to have the best activity on short chain fatty acids. Accordingly, this enzyme can be retargeted from the peroxisome to the cytoplasm by removing the peroxisomal targeting signal from its C-terminus.

To move hexanoyl-CoA along the engineered biosynthetic pathway of the invention, the genes TKS and OAC from C. sativa are preferably amplified in the yeast by integration into the genome of one or more copies driven by a fatty acid inducible promoter and/or a strong constitutive promoter. The pathway up to this point produces olivetolic acid from the fatty acid oleic acid (C18:1) as feedstock. Another target for engineering not shown on FIG. 1 is the enzyme acetyl-CoA carboxylase (ACC1), which produces malonyl-CoA for the TKS reaction. As the production of the Acc1p enzyme is tightly regulated in wild type strains, if this alternative is employed it is preferred to produce Acc1p not subject to tight wild-type regulation in order to produce sufficient amounts of malonyl-CoA for high production levels of olivetolic acid.

The gene encoding the enzyme activity geranylpyrophosphate:olivetolate geranyltransferase (e.g., CsPT1, NphB) is preferably similarly amplified by introduction of one or more additional copies into the production strain's genome. Because this enzyme may be an integral membrane protein, in some preferred embodiments of the invention the enzyme is targeted to the endoplasmic reticulum or other membranous organelle by engineering the desired signal peptide into the corresponding genetic construct. Yet another alternative is to use a truncated Pts1p that is soluble and resides in the cytoplasm. Still another option is to employ a different soluble heterologous enzyme with the appropriate prenyl transferase activity, for example, the Streptomyces NphB gene mentioned above.

Another target for engineering not shown in FIG. 1 is the mevalonate pathway, which can be engineered to produce the C10 molecule geranylpyrophosphate for the PTS1 reaction. This biochemical pathway is native to C. viswanathii and can be amplified by introducing extra copies of the pathway genes and/or by promoter replacement. Additionally, the enzyme encoded by the gene ERG20 normally produces the C15 molecule farnesyl pyrophosphate. Mutants of ERG20 or enzymes from heterologous sources can be used to increase the enzymatic activity producing geranyl pyrophosphate from the C5 substrates dimethylallyl pyrophosphate and isopentenyl pyrophosphate.

The final enzyme of the pathway, CBDA synthase (CBDAS), is also preferably amplified by engineering the production strain's genome to contain multiple copies of the corresponding gene. In C. sativa, the enzyme is directed to the secretory pathway where it appears to be glycosylated prior to secretion outside of the plant cell. Isolation of the enzyme from C. sativa resulted in a monomeric enzyme with apparent molecular mass of 75 kDa (Taura, et al., 1996). Similarly, the THCA synthase enzyme isolated from C. sativa demonstrated an apparent molecular mass of 75 kDa (Taura, et al., 1995). Subsequent cloning and expression of the CBDAS gene in insect cells resulted in an active enzyme with an apparent molecular mass of −62 kDa (the theoretical molecular weight of the mature enzyme is 59 kDa) (Taura et al., 2007). Cloning and expression of THCAS in insect cells and yeast also resulted in an active enzyme with apparent molecular weight lower than that isolated from C. sativa. In both of theses enzymes (CBDAS and THCAS), glycosylation does not appear to be required for activity.

The substrate and product for the CBDAS reaction may have toxicity in yeast, as is the case for plant cells. The toxicity of cannabigerolic acid can be avoided as long as there is sufficient activity of CBDAS in the cell. To avoid CBDA toxicity, a unique storage compartment in yeast is created, namely, the formation of lipid droplets inside yeast cells. Using fatty acids as feedstock allows yeast to produce lipid droplets without the cells having to expend the energy to make the fatty acids de novo. These lipid droplets can serve as storage compartments for hydrophobic molecules such as CBDA, thereby preventing toxicity by disrupting other membranous organelles.

### Feedstocks, Media, Supplements, and Additives

Engineered microorganisms are cultured under conditions that preferably optimize cannabinoid yield, often by optimizing activity of one or more of following enzymatic activities of a pathway engineered to produce hexanoyl-CoA or a desired or target cannabinoid. In general, non-limiting examples of conditions that may be optimized include the type and amount of carbon source, the type and amount of nitrogen source, the carbon-to-nitrogen ratio, the oxygen level, growth temperature, pH, length of the biomass production phase, length of target product accumulation phase, and time of cell and/or product harvest.

Culture media generally contain a suitable carbon source. Carbon sources useful for culturing microorganisms and/or fermentation processes sometimes are referred to as feedstocks. The term “feedstock” as used herein refers to a composition containing a carbon source that is provided to an organism, which is used by the organism to produce energy and metabolic products useful for growth. A feedstock may be a natural substance, a “man-made substance”, a purified or isolated substance, a mixture of purified substances, a mixture of unpurified substances, or combinations thereof. A carbon source can include one or more of the following substances: alkanes; alkenes, mono-carboxylic acids, di-carboxylic acids, monosaccharides (e.g., also referred to as “saccharides,” which include 6-carbon sugars (e.g., glucose, fructose), 5-carbon sugars (e.g., xylose and other pentoses) and the like), disaccharides (e.g., lactose, sucrose), oligosaccharides (e.g., glycans, homopolymers of a monosaccharide); polysaccharides (e.g., starch, cellulose, heteropolymers of monosaccharides or mixtures thereof), sugar alcohols (e.g., glycerol), and renewable feedstocks (e.g., cheese whey permeate, cornsteep liquor, sugar beet molasses, barley malt).

Carbon sources also can be selected from one or more of the following non-limiting examples: paraffin (e.g., saturated-paraffin, unsaturated paraffin, substituted paraffin, linear paraffin, branched paraffin, or combinations thereof); alkanes (e.g., dodecane), alkenes or alkynes, each of which may be linear, branched, saturated, unsaturated, substituted or combinations thereof (described in greater detail below); linear or branched alcohols (e.g., dodecanol); fatty acids (e.g., about 1 carbon to about 60 carbons, including free fatty acids such as, without limitation, caproic acid, capryllic acid, capric acid, lauric acid, myristic acid, palmitic acid, palmitoleic acid, stearic acid, oleic acid, linoleic acid, linolenic acid), or soap stock, for example; esters (such as methyl esters, ethyl esters, butyl esters, and the like) of fatty acids including, without limitation, esters such as methyl caprate, ethyl caprate, methyl laurate, ethyl laurate, methyl myristate, ethyl myristate, methyl caprolate, ethyl caprolate, ethyl caprillic, methyl caprillic, methyl palmitate, or ethyl palmitate; monoglycerides; diglycerides; triglycerides, phospholipids. Non-limiting commercial sources of products for preparing feedstocks include plants, plant oils or plant products (e.g., vegetable oils (e.g., almond oil, canola oil, cocoa butter, coconut oil, corn oil, cottonseed oil, flaxseed oil, grape seed oil, illipe, olive oil, palm oil, palm olein, palm kernel oil, safflower oil, peanut oil, soybean oil, sesame oil, shea nut oil, sunflower oil walnut oil, the like and combinations thereof) and animal fats (e.g., beef tallow, butterfat, lard, cod liver oil). A carbon source may included petroleum product and/or a petroleum distillate (e.g., diesel, fuel oils, gasoline, kerosene, paraffin wax, paraffin oil, petrochemicals). In some embodiments, a feedstock comprises petroleum distillate. A carbon source can be a fatty acid distillate (e.g., a palm oil distillate or corn oil distillate). Fatty acid distillates can be by-products from the refining of crude plant oils. In some embodiments, a feedstock comprises a fatty acid distillate.

In some embodiments, a feedstock comprises a soapstock (i.e., soap stock). A widely practiced method for purifying crude vegetable oils for edible use is the alkali or caustic refining method. This process employs a dilute aqueous solution of caustic soda to react with the free fatty acids present, which results in the formation of soaps. The soaps, together with hydrated phosphatides, gums, and prooxidant metals, are typically separated from the refined oil as the heavy phase discharge from the refining centrifuge and are typically known as soapstock.

A carbon source also may include a metabolic product that can be used directly as a metabolic substrate in an engineered pathway described herein, or indirectly via conversion to a different molecule using engineered or native biosynthetic pathways in an engineered microorganism. In certain embodiments, metabolic pathways can be preferentially biased towards production of a desired product by increasing the levels of one or more activities in one or more metabolic pathways having and/or generating at least one common metabolic and/or synthetic substrate.

In some embodiments a feedstock is selected according to the genotype and/or phenotype of the engineered microorganism to be cultured. For example, a feedstock rich in 12-carbon fatty acids can be useful for culturing certain yeast strains. Non-limiting examples of carbon sources having 10 to 14 carbons include fats (e.g., coconut oil, palm kernel oil), paraffins (e.g., alkanes, alkenes, or alkynes) having 10 to 14 carbons, (e.g., dodecane (also referred to as adakanel2, bihexyl, dihexyl and duodecane); tetradecane), alkene and alkyne derivatives), fatty acids (dodecanoic acid, tetradecanoic acid), fatty alcohols (dodecanol, tetradecanol), and non-toxic substituted derivatives or combinations thereof.

In some embodiments, a feedstock includes a mixture of carbon sources, where each carbon source in the feedstock is selected based on the genotype of the engineered microorganism. In certain embodiments, a mixed carbon source feedstock includes one or more carbon sources selected from sugars, cellulose, alkanes, fatty acids, triacylglycerides, paraffins, the like and combinations thereof.

Nitrogen may be supplied from an inorganic (e.g.; (NH₄)₂SO₄) or organic source (e.g., urea or glutamate). In addition to appropriate carbon and nitrogen sources; culture media also can contain suitable minerals, salts, cofactors, buffers, vitamins, metal ions (e.g., Mn⁺², Co⁺², Zn⁺², Mg⁺²) and other components suitable for culture of microorganisms.

Engineered microorganisms sometimes are cultured in complex media (e.g., yeast extract-peptone-dextrose broth (YPD)). In some embodiments, engineered microorganisms are cultured in a defined minimal media that lacks a component necessary for growth and thereby forces selection of a desired expression cassette (e.g., Yeast Nitrogen Base (DIFCO Laboratories, Detroit, Mich.)).

Culture media in some embodiments are common commercially prepared media, such as Yeast Nitrogen Base (DIFCO Laboratories, Detroit, Mich.). Other defined or synthetic growth media may also be used and the appropriate medium for growth of the particular microorganism are known.

Growth Conditions, Fermentation

A suitable pH range for the fermentation often is between about pH 4.0 to about pH 8.0, where a pH in the range of about pH 5.5 to about pH 7.0 sometimes is utilized for initial culture conditions. Depending on the engineered microorganism, culturing may be conducted under aerobic or anaerobic conditions, where microaerobic conditions sometimes are maintained. A two-stage process may be utilized, where one stage promotes microorganism proliferation and another stage promotes production of the desired product (or its precursor). In a two-stage process, the first stage may be conducted under aerobic conditions (e.g., introduction of air and/or oxygen) and the second stage may be conducted under anaerobic conditions (e.g., air or oxygen are not introduced to the culture conditions). In some embodiments, the first stage may be conducted under anaerobic conditions and the second stage may be conducted under aerobic conditions. In certain embodiments, a two-stage process may include two or more engineered microorganism types (or species), where one engineered microorganism type generates an intermediate product in one stage and another engineered microorganism type processes the intermediate product into a desired product, e.g., cannabigerolic acid or CBD in another stage, for example.

A variety of fermentation processes may be applied for commercial biosynthetic cannabinoid production in accordance with the invention. In some embodiments, commercial production of cannabinoid from a recombinant, engineered microbial host is conducted using a batch, fed-batch, or continuous fermentation process, for example.

A batch fermentation processes often use a closed system where the media composition is fixed at the beginning of the process and not subject to further additions beyond those required for maintenance of pH and oxygen level during the process. At the beginning of the culturing process the media is inoculated with the desired cannabinoid-producing, engineered microorganism type(s) and growth or metabolic activity is permitted to occur without adding additional sources (i.e., carbon and nitrogen sources) to the medium. In batch processes the metabolite and biomass compositions of the system change constantly up to the time the culture is terminated. In a typical batch process, cells proceed through a static lag phase to a high-growth log phase and finally to a stationary phase, wherein the growth rate is diminished or halted. Left untreated, cells in the stationary phase will eventually die.

A variation of the standard batch process is the fed-batch process, where the carbon source(s) is(are) continually added to the fermenter over the course of the fermentation run. Fed-batch processes are useful when catabolite repression is apt to inhibit the metabolism of the cells or where it is desirable to have limited amounts of carbon source in the media at any one time. Measurement of the carbon source concentration in fed-batch systems may be estimated on the basis of the changes of measurable factors such as pH, dissolved oxygen and the partial pressure of waste gases (e.g., 002).

Batch and fed-batch culturing methods are known in the art. Examples of such methods may be found in Thomas D. Brock in Biotechnology: A Textbook of Industrial Microbiology, 2.sup.nd ed., (1989) Sinauer Associates Sunderland, Mass. and Deshpande, Mukund V., Appl. Biochem. Biotechnol., 36:227 (1992).

In a continuous fermentation process, a defined media often is continuously added to a bioreactor while an equal amount of culture volume is removed simultaneously for product recovery. Continuous cultures generally maintain cells in the log phase of growth at a constant cell density. Continuous or semi-continuous culture methods permit the modulation of one factor or any number of factors that affect cell growth or end product concentration. For example, an approach may limit the carbon source(s) and allow all other parameters to moderate metabolism. In some systems, a number of factors affecting growth may be altered continuously while the cell concentration, measured by media turbidity, is kept constant. Continuous systems often maintain steady state growth and thus the cell growth rate often is balanced against cell loss due to media being drawn off the culture. Methods of modulating nutrients and growth factors for continuous culture processes, as well as techniques for maximizing the rate of product formation, are known and a variety of methods, are detailed by Brock, supra.

In some embodiments involving fermentation, the fermentation can be carried out using two or more types of engineered microorganisms (e.g., host microorganism, engineered microorganism, isolated naturally occurring microorganism, the like and combinations thereof), where a feedstock is partially or completely utilized by one or more organisms in the fermentation (e.g., mixed fermentation), and the products of cellular respiration or metabolism of one or more microorganism types can be further metabolized by engineered microorganisms according to the invention to produce a desired product, for example, CBD. In certain embodiments, each microorganism type can be fermented independently and the products of cellular respiration or metabolism purified and contacted with an engineered microorganism of the invention to produce cannabidiol. Any suitable combination of microorganisms can be utilized to carry out mixed fermentation or sequential fermentation.

### Enhanced Fermentation Processes

It has been known that certain feedstock components are toxic to, or produce a by-product (e.g., metabolite) that can be toxic to, for example, yeast utilized in a fermentation process for the purpose of producing a desired product, although a toxic component or metabolite from a feedstock can sometimes be utilized by an engineered microorganism to produce the desired product.

For example, in some instances a fatty acid component having 12 or fewer carbons can be toxic to yeast. Components that are not free fatty acids, but are processed by yeast to a fatty acid having twelve or fewer carbons, also may have a toxic effect. Non-limiting examples of such components are esters of fatty acids (e.g., methyl esters, monoglycerides, diglycerides, triglycerides) that are processed by yeast into a fatty acid having twelve or fewer carbons. Feedstocks containing molecules that are directly toxic, or indirectly toxic by conversion of a nontoxic component to a toxic metabolite, are collectively referred to as “toxic feedstocks” and “toxic components.” Providing an engineered microorganism with a feedstock that comprises or delivers one or more toxic components can reduce the viability of the engineered microorganism and/or reduce the amount of desired product produced thereby.

In some embodiments, a process for overcoming the toxic effect of certain components in a feedstock includes first inducing the engineered microorganism with a feedstock not containing a substantially toxic component and then providing the engineered microorganism with a feedstock that comprises a toxic component. In some embodiments, the second feedstock is provided for a certain amount of time (e.g., about 1 to about 48 hours, inclusive).

### Cannabinoid Production, Isolation, and Yield

In various embodiments the cannabinoid product is isolated or purified from the culture media or extracted from the engineered microorganisms. In some embodiments, fermentation of feedstocks by methods described herein can produce the cannabinoid product at a level of about 1% to about 100% of theoretical yield (inclusive of all percentages and ranges of percentages bounded thereby). The term “theoretical yield” refers to the amount of product that could be made from a starting material if the reaction is 100% efficient. Theoretical yield is based on the stoichiometry of a reaction and ideal conditions in which starting material is completely consumed, undesired side reactions do not occur, the reverse reaction does not occur, and there are no losses in the workup procedure. Culture media may be tested for the cannabinoid product concentration and drawn off when the concentration reaches a predetermined level. Detection methods are known in the art, including but not limited to chromatographic methods (e.g., gas chromatography) or combined chromatographic/mass spectrometry (e.g., GC-MS) methods.

The cannabinoid product sometimes is retained within an engineered microorganism after a culture process is completed, and in certain embodiments, the cannabinoid product may be secreted out of or otherwise released from the microorganism into the culture medium. For the latter embodiments, (i) culture media may be drawn from the culture system and fresh medium may be supplemented, and/or (ii) the cannabinoid product may be extracted from the culture media during or after the culture process is completed. Engineered microorganisms may be cultured on or in solid, semi-solid, or liquid media. In some embodiments, media is drained from cells adhering to a plate. In certain embodiments, a liquid-cell mixture is centrifuged at a speed sufficient to pellet the cells but not disrupt the cells and allow extraction of the media, as known in the art. The cells may then be resuspended in fresh media. The cannabinoid product may be purified from culture media by any suitable method.

In some embodiments, the cannabinoid product is present in a product containing other byproducts. The cannabinoid product can be purified from the other byproducts using a suitable purification procedure. Partially purified or substantially purified cannabinoid may be produced using a purification process.

In some embodiments, the cannabinoid product is extracted from cultured engineered microorganisms. The microorganism cells may be concentrated through centrifugation at a speed sufficient to shear the cell membranes. In some embodiments, the cells may be physically disrupted (e.g., shear force, sonication) or chemically disrupted (e.g., contacted with detergent or other lysing agent). The phases may be separated by centrifugation or other method known in the art and target product may be isolated according to known methods.

Commercial grade cannabinoids such as CBD sometimes are provided in substantially pure form (e.g., 90% pure or greater, 95% pure or greater, 99% pure or greater or 99.5% pure or greater). In some embodiments, CBD may be modified into any one of a number of downstream products.

In preferred embodiments, the cannabinoid yield is about 0.001 g/L to about 100 g/L (inclusive of all yields, and subsets of ranges, bounded thereby).

### EXAMPLES

The invention will be further described by reference to the following detailed Examples, which are in no way to be considered to limit the scope of the invention.

### Example 1: Generation of a Ura⁻ Mutant of Candida Strain ATCC20962 (ura3/ura3 pox4a::ura3/pox4b::ura3 pox5::ura3/pox5::ura3)

Candida strain ATCC20962 (see U.S. Pat. No. 5,254,466) is a beta-oxidation blocked (Pox4⁻, Pox5⁻) and Um′ derivative of Candida strain ATCC20336. See, e.g., U.S. Pat. No. 8,241,879. To reutilize the URA3 marker for subsequent engineering, a single colony having the Ura⁺ phenotype was inoculated into 3 mL YPD and grown overnight at 30° C. with shaking. The overnight culture was then harvested by centrifugation and resuspended in 1 mL YNB+YE (6.7 g/L Yeast Nitrogen Broth, 3 g/L Yeast Extract). The resuspension was then serially diluted in YNB+YE and 100 uL aliquots plated on YPD plates (incubation overnight at 30° C.) to determine the titer of the original suspension. Additionally, triplicate 100 uL aliquots of the undiluted suspension were plated on SC Dextrose (Bacto Agar 20 g/L, Uracil 0.3 g/L, Dextrose 20 g/L, Yeast Nitrogen Broth 6.7 g/L, Amino Acid Dropout Mix 2.14 g/L) and 5-FOA at 3 different concentrations (0.5, 0.75, and 1 mg/mL). Plates were incubated for at least 5 days at 30° C. Colonies arising on the SC Dextrose+5-FOA plates were picked into 50 uL sterile, distilled water and 5 uL struck out to YPD and SC-URA (SC Dextrose medium without Uracil). Colonies growing only on YPD and not on SC-URA plates were then inoculated into 3 mL YPD and grown overnight at 30° C. with shaking. The overnight culture was then harvested by centrifugation and resuspended in 1.5 mL YNB (6.7 g/L Yeast Nitrogen Broth). The resuspension was then serially diluted in YNB and 100 uL aliquots plated on YPD plates (incubation overnight at 30° C.) to determine the initial titer. Also, for each undiluted suspension, 1 mL was plated on SC-URA and incubated for up to 7 days at 30° C. Colonies on the SC-URA plates were revertants, and the isolate with the lowest reversion frequency (<10⁻⁷) was named sAA0103 and used for subsequent strain engineering.

### Example 2: Generation of a Ura⁻Leu⁻ Mutant of Candida Strain ATCC20962 (ura3/ura3 pox4a::ura3/pox4b::ura3 pox5::ura3/pox5::ura3 leu2-Δ1::Tᵤᵣₐ₃/leu2-Δ2::Tᵤᵣₐ₃)

The LEU2 gene and flanking DNA sequence was amplified by PCR from ATCC20336 genomic DNA to produce an amplicon that was then gel-purified and cloned into plasmid pCR-BluntII-TOPO (Invitrogen). A sequence-confirmed plasmid with the correct construction was saved as plasmid pAA2333.

In order to knock out the first copy of LEU2 from the genome, plasmid pAA3060 with a 259 bp and a 256 bp homology region to both the 5′ and 3′ region, respectively, of the LEU2 gene was constructed with a URA3 selection cassette. The 5′ region was amplified with primers oAA7682 and oAA7683, the 3′ region was amplified with oAA7686 and oAA7687, the URA3 cassette was amplified from pAA244 using oAA7684 and oAA7685, and all pieces were assembled by overlap PCR and cloned into pCR-BluntII-TOPO (Invitrogen), generating plasmid pAA3060.

Plasmid pAA3060 was then digested with BamHI and PstI and the resulting fragment was integrated into the Ura⁻ version of Candida strain ATCC 20962 genome with URA selection. One transformant with the correct genome modification was saved as strain sAA6404 (leu2-Δ1::URA3/LEU2). Strain sAA6404 was plated onto 5′FOA for the loop-out of URA3, leaving a URA3 terminator scar. One colony identified with the correct genome modification was saved as strain sAA6462 (leu2-Δ1::T_{URA3}/LEU2).

In order to knock out the second copy of LEU2 from the genome, plasmid pAA2417 with a 204 bp and a 283 bp homology region to both the 5′ and 3′ region respectively of the LEU2 gene was constructed with a URA3 selection cassette. The 5′ region was amplified with oAA7941 and oAA7942, the 3′ region was amplified with oAA7945 and oAA7946, the URA3 cassette was amplified from pAA244 using oAA7943 and oAA7944, and all pieces were assembled by overlap PCR and cloned into pCR-BluntII-TOPO (Invitrogen), generating plasmid pAA2417. Plasmid pAA2417 was then digested with BamHI and PstI and the resulting fragment was integrated into the sAA6462 genome with URA selection. One correct transformant was identified and saved as strain sAA6860 (leu2-Δ1::T_{URA3}/leu2-Δ2::URA3). Strain sAA6860 was plated onto 5′FOA for the loop-out of URA3, leaving a URA3 terminator scar. One colony with the correct genome modification was identified and saved as strain sAA7790 (leu2-A1::T_{URA3}/leu2,42::T_{URA3}).

### Example 3: Acyl-CoA Oxidase (ACO1) from Arthrobacter ureafaciens Cloning

A DNA sequence codon-optimized for Candida viswanathii encoding the acyl-CoA oxidase enzyme (EC #1.3.3.6) from A. ureafaciens was synthesized as gBlocks (IDT) and assembled by overlap PCR with oligos oAA3491/oAA3492. The resulting 2,019 bp amplicon was cloned into pCR-BluntII-TOPO (Invitrogen). A sequence-verified plasmid with the correct construction was saved as plasmid pAA873. The DNA sequence encoding AuAco1p was amplified by PCR using pAA873 as template and oligos oAA3458/oAA3750 that introduce BspQI restriction sites and a C-terminal tripeptide (AKL) for peroxisomal targeting. The amplicon was gel-purified and cloned into pCR-BluntII-TOPO (Invitrogen). A sequence-verified plasmid with the correct construction was saved as plasmid pAA956. Plasmid pAA956 was cut with BspQI and the 2,124 bp DNA fragment encoding AuAco1p with C-terminal AKL tripeptide was gel-purified and ligated into BspQI-cut plasmid pAA335. A sequence-verified plasmid with the correct construction was saved as plasmid pAA964.

### Example 4: Polyketide Synthase (PKS) Cloning

A DNA sequence codon-optimized for Candida viswanathii encoding the polyketide synthase enzyme (EC #2.3.1.206) from Cannabis sativa was synthesized as a gBlock (IDT) including a C-terminal 6×His tag. The 1,176 bp gBlock was cloned into pCR-BluntII-TOPO (Invitrogen). A sequence-verified plasmid with the correct construction was saved as plasmid pVZ3970. In order to place the PKS gene, without the C-terminal 6×His tag, under the control of the HDE promoter and PDX4 terminator, the PKS gene was amplified by PCR with oligos oVZ153/oVZ154 (1,208 bp amplicon) and plasmid pAA1164 was amplified by PCR with oligos oVZ151/oVZ152 (5,964 bp amplicon) and the two amplicons were assembled by directional ligation. A sequence-verified plasmid with the correct construction was saved as plasmid pVZ4009.

### Example 5: Olivetolic Acid Cyclase (OAC) Cloning

A DNA sequence codon-optimized for Candida viswanathii encoding the olivetolic acid cyclase enzyme (EC #4.4.1.26) from Cannabis sativa was synthesized as a gBlock (IDT) including a C-terminal 6×His tag. The 324 bp gBlock was cloned into pCR-BluntII-TOPO (Invitrogen). A sequence-verified plasmid with the correct construction was saved as plasmid pVZ3968. In order to place the OAC gene, without the C-terminal 6×His tag, under the control of the HDE promoter and PDX4 terminator, the OAC gene was amplified by PCR using pVZ3968 as template with oligos oVZ155/oVZ156 (356 bp amplicon) and plasmid pAA1164 was amplified by PCR with oligos oVZ151/oVZ152 (5,964 bp amplicon) and the two amplicons were assembled by directional ligation. A sequence-verified plasmid with the correct construction was saved as plasmid pVZ4008.

### Example 6: Acyl-CoA Synthetase (ACS2d) Cloning

The DNA sequence encoding the acyl-CoA synthetase enzyme ACS2d (EC #6.2.1.2) from Candida strain ATCC20336 was amplified from gDNA with 5′ and 3′ flanking sequence using oligos oAA4966/oAA4967 producing a 2,896 bp amplicon that was cloned into pCR-BluntII-TOPO (Invitrogen). A sequence confirmed plasmid with the correct construction was named pAA1417.

A plasmid with the Candida viswanathii LEU2 selectable marker was generated by first constructing a modified pUC19 vector. Oligos oAA4394/oAA4395 were annealed together and ligated to pUC19 cut with NdeI and HindIII. A sequence-verified plasmid of the correct construction was saved as plasmid pAA1222.

Plasmid pAA1222 was amplified by PCR with oligos oVZ337/oVZ338 producing an amplicon of 2,442 bp. Candida strain ATCC20336 gDNA was used as a template for PCR with two pairs of oligos, oVZ339/oVZ340 and oVZ341/oVZ342, to produce amplicons of 767 bp and 1,237 bp, respectively. These three linear DNA fragments were gel-purified and assembled by directional ligation. A sequence-verified plasmid of the correct construction was saved as plasmid pVZ4045. This plasmid provides a split LEU2 selectable marker ready for the insertion of promoter-gene-terminator combinations.

The ACS2d gene sequence was cloned into plasmid pVZ4045 under the control of the HDE (751 bp) promoter and PDX4 terminator (174 bp) by directional ligation. A sequence-verified plasmid of the correct construction was saved as plasmid pVZ4285.

The DNA sequence encoding the three C-terminal residues of the ACS2d gene was removed from plasmid pVZ4285 to result in a plasmid expressing an enzyme without a peroxisomal targeting sequence (ACS2d^{Δpts}) for targeting to the cytoplasm. Plasmid pVZ4285 was used as template in PCR with the 5′ phosphorylated oligos oVZ1117/oVZ1118 producing an amplicon of 7,509 bp that was gel-purified and ligated with T4 DNA ligase. A sequence-verified plasmid of the correct construction was saved as plasmid pVZ4348.

### Example 7: Acyl Activating Enzyme (AAE1) Cloning

A DNA sequence codon-optimized for Candida viswanathii encoding the acyl-activating enzyme (EC #6.2.1.2) from Cannabis sativa was synthesized as a gBlock (IDT). The 2,163 bp gBlock was cloned into pCR-BluntII-TOPO (Invitrogen). A sequence-verified plasmid with the correct sequence was saved as plasmid pVZ4277. The AAE1 gene was cloned into plasmid pVZ4045 under the control of the HDE promoter and PDX4 terminator by directional ligation. A sequence-verified plasmid with the correct sequence was saved as plasmid pVZ4282.

### Example 8: Farnesyl Diphosphate Synthase (ERG20) Cloning

The DNA sequence encoding the farnesyl diphosphate synthase enzyme ERG20 (EC #2.5.1.1, 2.5.1.10) from Candida strain ATCC20336 was amplified from genomic DNA and cloned into plasmid pVZ4045 under the control of the PDX18 promoter (360 bp) and PDX18 terminator (374 bp). A sequence-verified plasmid of the correct construction was saved as plasmid pVZ4105. An ERG20(F95W,N126W) mutant was constructed by directional cloning of gBlock (IDT) bRB001 (125 bp) and pVZ4105 amplified with primers oRB008/oRB009 (6,082 bp). A sequence-verified plasmid with the correct construction was saved as plasmid pRB0076.

### Example 9: Geranylpyrophosphate-Olivetolic Acid Transferase (GOT) Cloning

A DNA sequence codon-optimized for Candida viswanathii encoding the CsPT1 geranylpyrophosphate-olivetolic acid transferase enzyme (EC #2.5.1.102) from Cannabis sativa was synthesized as a gBlock (IDT). The 1,188 bp gBlock was cloned into pCR-BluntII-TOPO (Invitrogen). A sequence-verified plasmid with the correct construction was saved as plasmid pAA3169. In order to place the CsPT1 gene under the control of the GPD promoter and PDX4 terminator, the CsPT1 gene was amplified by PCR using pAA3169 as template with oligos oAA9865/oAA9868 (1,229 bp amplicon) and plasmid pAA1922 was amplified by PCR with oligos oAA9863/oAA9864 (5,827 bp amplicon). The two amplicons were gel-purified and assembled by directional ligation. A sequence-verified plasmid with the correct construction was saved as plasmid pAA3636.

The DNA sequence encoding CsPT1 was cloned into plasmid pVZ4045 under the control of the HDE promoter and PDX4 terminator by directional ligation. A sequence-verified plasmid with the correct construction was saved as plasmid pRB0073.

The DNA sequence encoding CsPT1 without its N-terminal signal sequence was cloned into plasmid pVZ4045 under the control of the HDE promoter and PDX4 terminator by directional ligation. A sequence-verified plasmid with the correct construction was saved as plasmid pRB0074.

A DNA sequence codon-optimized for Candida viswanathii encoding the CsPT4 geranylpyrophosphate-olivetolic acid transferase enzyme (EC #2.5.1.102) from Cannabis sativa was synthesized as a gBlock (IDT). The DNA sequence encoding CsPT4 without its N-terminal signal sequence was cloned into plasmid pVZ4045 under the control of the HDE promoter and PDX4 terminator by directional ligation. A sequence-verified plasmid with the correct construction was saved as plasmid pRB0077.

A DNA sequence codon-optimized for Candida viswanathii encoding the NphB(G286S,Y288A) aromatic prenyltransferase enzyme (EC #2.5.1.102) from Streptomyces sp. was synthesized as a gBlock (IDT). The DNA sequence encoding NphB(G286S,Y288A) was cloned into plasmid pVZ4045 under the control of the HDE promoter and PDX4 terminator by directional ligation. A sequence-verified plasmid with the correct construction was saved as plasmid pRB0085.

### Example 10: Cannabidiolic Acid Synthase (CBDAS) Cloning

A DNA sequence codon-optimized for Candida viswanathii encoding the cannabidiolic acid synthase enzyme without the putative signal sequence (EC #1.21.3.8) from Cannabis sativa was synthesized as a gBlock (IDT). The 1,554 bp gBlock was cloned into pCR-BluntII-TOPO (Invitrogen). A sequence-verified plasmid with the correct construction was saved as plasmid pAA3171. In order to place the CBDAS (no signal sequence) gene under the control of the GPD promoter and PDX4 terminator, the CBDAS (no signal sequence) gene was amplified by PCR using plasmid pAA3171 as template with oligos oAA9869/oAA9872 (1,595 bp amplicon) and plasmid pAA1922 was amplified by PCR with oligos oAA9863/oAA9864 (5,827 bp amplicon). The two amplicons were gel-purified and assembled by directional ligation. A sequence-verified plasmid with the correct construction was saved as plasmid pAA3632. The DNA sequence encoding the CBDAS signal sequence was incorporated by PCR amplification with the 5′-phosphorylated oligos oVZ331/oVZ332 using plasmid pAA3632 as template producing an amplicon of 7,462 bp that was gel-purified and ligated with T4 DNA ligase. A sequence-verified plasmid of the correct construction was saved as plasmid pVZ4124.

The DNA sequence encoding CBDAS without its N-terminal signal sequence was cloned into plasmid pVZ4045 under the control of the HDE promoter and PDX4 terminator by directional ligation. A sequence-verified plasmid with the correct construction was saved as plasmid pRB0075.

### Example 11: Cannabichromenic Acid Synthase (CBCAS) Cloning

A DNA sequence codon-optimized for Candida viswanathii encoding the cannabichromenic acid synthase enzyme without the putative signal sequence (EC #1.21.3.-) from Cannabis sativa was synthesized as a gBlock (IDT). The DNA sequence encoding CBCAS without its N-terminal signal sequence was cloned into plasmid pVZ4045 under the control of the HDE promoter and PDX4 terminator by directional ligation. A sequence-verified plasmid with the correct construction was saved as plasmid pRB0084.

### Example 12: Construction of Yeast with Engineered Pathway to Olivetolic Acid from Hexanoic Acid

Strains sAA103 was transformed with two linear DNA constructs encoding the PKS and OAC enzymes from C. sativa. The PKS construct was generated by PCR amplification using plasmid pVZ4009 as template with oligos oAA2206/oAA2209 producing a 3,603 bp amplicon containing the PKS gene under the control of the HDE promoter and PDX4 terminator as well as a URA3 marker. The OAC construct was generated by PCR amplification using plasmid pVZ4008 as template with oligos oAA2206/oAA2209 producing a 2,751 bp amplicon containing the OAC gene under the control of the HDE promoter and PDX4 terminator as well as a URA3 marker. All linear DNA constructs were gel-purified prior to transformation of strain sAA103 and plating on SC-URA media. Transformants were screened by PCR for the presence of both DNA constructs and one transformant with the correct construction was saved as strain sAA9712.

### Example 13: Construction of Yeast with an Engineered Pathway to Olivetolic Acid from Fatty Acids

Strain sAA7790 was transformed with three linear DNA constructs encoding the PKS and OAC enzymes from C. sativa and the AC01 enzyme from A. ureafaciens. The PKS construct was generated by PCR amplification using plasmid pVZ4009 as template with oligos oAA2206/oAA2209 producing a 3,603 bp amplicon containing the PKS gene under the control of the HDE promoter and PDX4 terminator as well as a URA3 marker. The OAC construct was generated by PCR amplification using plasmid pVZ4008 as template with oligos oAA2206/oAA2209 producing a 2,751 bp amplicon containing the OAC gene under the control of the HDE promoter and PDX4 terminator as well as a URA3 marker. The AuACO1 construct was generated by linearization of plasmid pAA964 with Drain producing a 6,391 bp fragment containing the AuACO1 gene under the control of the PEX11 promoter and terminator as well as a URA3 marker. All linear DNA constructs were gel-purified prior to transformation of strain sAA7790 and plating on SC-URA media. Transformants were screened by PCR for the presence of all three DNA constructs and three transformants with the correct construction were saved as strains sAA9920, sAA9921, and sAA9922.

Strain sAA9920 was further transformed with DNA constructs encoding acyl-CoA synthetase enzymes. An ACS2d construct was generated by PCR amplification using plasmid pVZ4285 as template with oligos oVZ0373/oVZ0374 producing an 5,074 bp amplicon containing the ACS2d gene under the control of the HDE promoter and PDX4 terminator as well as a LEU2 marker. The amplicon was gel-purified and transformed into strain sAA9920 and plated on SC-LEU media. Transformants were screened by PCR for the presence of the ACS2d construct and four transformants with the correct construction were saved as strains sVZ0070, sVZ0071, sVZ0072, and sVZ0073.

An AAE1 construct was generated by PCR amplification using plasmid pVZ4282 as template with oligos oVZ0373/oVZ0374 producing an 5,014 bp amplicon containing the AAE1 gene under the control of the HDE promoter and PDX4 terminator as well as a LEU2 marker. The amplicon was gel-purified and transformed into strain sAA9920 and plated on SC-LEU media. Transformants were screened by PCR for the presence of the AAE1 construct and four transformants with the correct construction were saved as strains sVZ0074, sVZ0075, sVZ0076, and sVZ0077.

An ACS2d^{Δpts} construct was generated by PCR amplification using plasmid pVZ4348 as template with oligos oVZ0373/oVZ0374 producing an 5,065 bp amplicon containing the ACS2d^{Δpts} gene under the control of the HDE promoter and PDX4 terminator as well as a LEU2 marker. The amplicon was gel-purified and transformed into strain sAA9920 and plated on SC-LEU media. Transformants were screened by PCR for the presence of the ACS2d^{Δpts} construct and four transformants with the correct construction were saved as strains sVZ0206, sVZ0207, sVZ0208, and sVZ0209.

### Example 14: Construction of Yeast with Engineered Pathway to Cannabigerolic Acid from Fatty Acids

Strain sAA9920 was transformed with DNA constructs encoding acyl-CoA synthetase (CsAAE1), geranylpyrophosphate-olivetolic acid transferase [CsPT1-noSS, CsPT4-noSS, or NphB(G286S,Y288A)], and farnesyl diphosphate synthase [ERG20(F95W,N126W)] according to the table below. DNA transformation cassettes targeting the LEU2 locus were amplified by PCR using oligos oRB0010/oRB0011 and plasmid DNA template. All amplicons were gel-purified and transformed into strain sAA9920 and plated on SC-LEU media. Transformants were screened by PCR for the presence of each transformed cassette and those with the correct construction (strains sRB008, sRB010, and sRB018) were saved as strains shown in the table below.

TABLE 1
Construction of Engineered Yeast with CBGA Pathway

| | | Cassette | | | |
|---|---|---|---|---|---|
| | | size | | | |
| Gene | Plasmid | (bp) | sRB008 | sRB010 | sRB018 |
| CsPT1-noSS | pRB0073 | 3,826 | + | | |
| CsPT4-noSS | pRB0077 | 3,830 | | + | |
| NphB(G286S, | pRB0085 | 3,785 | | | + |
| Y288A) | | | | | |
| ERG20(F95W, | pRB0076 | 3,716 | + | + | + |
| N126W | | | | | |
| CsAAE1 | pVZ4282 | 5,014 | + | + | + |

### Example 15: Construction of Yeast with Engineered Pathway to Cannabidiolic Acid from Fatty Acids

Strain sAA9920 was transformed with DNA constructs encoding acyl-CoA synthetase (CsAAE1), geranylpyrophosphate-olivetolic acid transferase [CsPT1-noSS, CsPT4-noSS, or NphB(G286S,Y288A)], farnesyl diphosphate synthase [ERG20(F95W,N126W)], and cannabidiolic acid synthase (CsCBDAS-noSS) according to the table below. DNA transformation cassettes targeting the LEU2 locus were amplified by PCR using oligos oRB0010/oRB0011 and plasmid DNA template. All amplicons were gel-purified and transformed into strain sAA9920 and plated on SC-LEU media. Transformants were screened by PCR for the presence of each transformed cassette and those with the correct construction (sRB009, sRB024, sRB025, sRB026, sRB027, and sRB028) were saved as strains shown in the table below.

TABLE 2
Construction of Engineered Yeast with CBDA Pathway

| | | Cassette | | | |
|---|---|---|---|---|---|
| | | size | | sRB024- | sRB027, |
| Gene | Plasmid | (bp) | sRB009 | sRB026 | sRB028 |
| CsPT1-noSS | pRB0073 | 3,826 | + | | |
| CsPT4-noSS | pRB0077 | 3,830 | | + | |
| NphB(G286S, | pRB0085 | 3,785 | | | + |
| Y288A) | | | | | |
| ERG20(F95W, | pRB0076 | 3,716 | + | + | + |
| N126W | | | | | |
| CsAAE1 | pVZ4282 | 5,014 | + | + | + |
| CsCBDAS-noSS | pRB0075 | 4,415 | + | + | + |

### Example 16: Basic Shake Flask Protocol

This example describes a basic shake flask protocol useful for the biosynthetic production of various compounds in the CBD biosynthetic pathway using engineered yeast strains and recombinant constructs according to the invention. Here, 250 mL glass flasks containing 50 mL of rich media (yeast nitrogen base, 6.7 g/L; yeast extract, 3.0 g/L; ammonium sulfate, 3.0 g/L; potassium phosphate monobasic, 1.0 g/L; potassium phosphate dibasic, 1.0 g/L; glycerol, 75 g/L) were inoculated with a 5 mL YPD overnight culture to an initial OD_{600 nm} of 0.4. After 24 h incubation at 30° C. with shaking at 250 rpm, the cells were centrifuged and the cell pellet resuspended in 15 mL of HiP-TAB media (yeast nitrogen base without amino acids and without ammonium sulfate, 1.7 g/L; yeast extract, 3.0 g/L; potassium phosphate monobasic, 10.0 g/L; potassium phosphate dibasic, 10.0 g/L). The cultures were transferred to fresh 250 mL glass bottom-baffled flasks and 2% (v/v) oleic acid was added. Cultures were incubated at 30° C. with shaking at 300 rpm. Samples were taken every 24 hours for gas chromatographic (GC) analysis and/or HPLC analysis.

### Example 17: Production of Hexanoic Acid in Strains sAA2380-sAA2383

Plasmid pAA964 was linearized by restriction digestion with DraIII and the resulting 6,391 bp linear DNA was gel-purified. The linearized plasmid was transformed into strain sAA0103 and plated onto YNB-hexadecane media to select for transformants with restored beta-oxidation. Colonies growing on YNB-hexadecane were then streaked onto SC-URA media to confirm the presence of the URA3 marker. The integration of the linearized DNA into the genome was confirmed by PCR and four colonies with the correct construction were saved as strains sAA2380, sAA2381, sAA2382, and sAA2383. The four strains were carried through shake flask characterization in duplicate using oleic acid as feedstock. Samples were analyzed by GC-FID for fatty acid production. Results for the 48-hour time point are shown in the table below.

TABLE 3
Hexanoic Acid in Engineered Strains

| Strain | Hexanoic acid (g/L) |
|---|---|
| SAA2380 flask 1 | 1.40 |
| SAA2380 flask 2 | 1.37 |
| SAA2381 flask 1 | 1.37 |
| SAA2381 flask 2 | 1.28 |
| SAA2382 flask 1 | 1.44 |
| SAA2382 flask 2 | 1.43 |
| SAA2383 flask 1 | 1.46 |
| SAA2383 flask 2 | 1.40 |

### Example 18: Production of Olivetolic Acid from Hexanoic Acid in Strain sAA9712

The engineered strain sAA9712 (Example 10, above) is beta-oxidation blocked with the introduction of genes for olivetolic acid synthase and olivetolic acid cyclase. It was investigated for its ability to produce olivetolic acid in shake flask testing (see, e.g., Example 14, above) with changes to the basic protocol, as follows. 250 mL glass flasks containing 50 mL of rich media (yeast nitrogen base, 6.7 g/L; yeast extract, 3.0 g/L; ammonium sulfate, 3.0 g/L; potassium phosphate monobasic, 1.0 g/L; potassium phosphate dibasic, 1.0 g/L; glycerol, 75 g/L) were inoculated with a 5 mL YPD overnight culture to an initial OD_{600 nm} of 0.4. After 24 h incubation at 25° C. with shaking at 250 rpm, the cells were centrifuged and the cell pellet resuspended in 15 mL of SC-Glycerol media (yeast nitrogen base, 6.7 g/L; synthetic complete mix, 2.1 g/L; glycerol, 20 g/L). The cultures were transferred to fresh 250 mL glass bottom-baffled flasks with and without supplementation of 1 mM hexanoic acid. Cultures were incubated at 25° C. with shaking at 300 rpm. Samples were taken at 24 and 120 hours for GC-MS analysis. At both time points, MS analysis confirmed olivetolic acid production in strain sAA9712 only in flasks supplemented with hexanoic acid. A control strain (beta-oxidation blocked without addition of the OAS and OAC genes) did not produce olivetolic acid.

### Example 19: Production of Olivetolic Acid from Oleic Acid in Strains sVZ0070-sVZ0077

Strains containing an engineered beta-oxidation pathway and an engineered pathway for the production of olivetolic acid (strains sVZ0070-sVZ0077; Example 11, above) were investigated for their ability to produce olivetolic acid in shake flask testing with slight modifications to the basic protocol (Example 14, above). 250 mL glass flasks containing 50 mL of rich media (yeast nitrogen base, 6.7 g/L; yeast extract, 3.0 g/L; ammonium sulfate, 3.0 g/L; potassium phosphate monobasic, 1.0 g/L; potassium phosphate dibasic, 1.0 g/L; glycerol, 75 g/L) were inoculated with a 5 mL YPD overnight culture to an initial OD_{600 nm} of 0.4. After 24 h incubation at 25° C. with shaking at 250 rpm, the cells were centrifuged and the cell pellet resuspended in 15 mL of HiP-TAB media (yeast nitrogen base without amino acids and without ammonium sulfate, 1.7 g/L; yeast extract, 3.0 g/L; potassium phosphate monobasic, 10.0 g/L; potassium phosphate dibasic, 10.0 g/L). The cultures were transferred to fresh 250 mL glass bottom-baffled flasks and 2% (v/v) oleic acid was added. Cultures were incubated at 30° C. with shaking at 300 rpm. Samples were taken at 120 hours for GC-MS analysis. In all samples, MS analysis confirmed the production of olivetolic acid.

### Example 20: Production of Olivetolic Acid from Oleic Acid with Glycerol Supplementation

Strains containing an engineered beta-oxidation pathway and an engineered pathway for the production of olivetolic acid (strains sVZ0070-sVZ0077; Example 11, above) were investigated for their ability to produce olivetolic acid in shake flask testing with slight modifications to the basic protocol (Example 14, above). The control strain sAA2382 (Example 14, above) with only an engineered beta-oxidation pathway was included. 250 mL glass flasks containing 50 mL of rich media (yeast nitrogen base, 6.7 g/L; yeast extract, 3.0 g/L; ammonium sulfate, 3.0 g/L; potassium phosphate monobasic, 1.0 g/L; potassium phosphate dibasic, 1.0 g/L; glycerol, 75 g/L) were inoculated with a 5 mL YPD overnight culture to an initial OD_{600 nm} of 0.4. After 24 h incubation at 25° C. with shaking at 250 rpm, the cells were centrifuged and the cell pellet resuspended in 15 mL of HiP-TAB media (yeast nitrogen base without amino acids and without ammonium sulfate, 1.7 g/L; yeast extract, 3.0 g/L; potassium phosphate monobasic, 10.0 g/L; potassium phosphate dibasic, 10.0 g/L). The cultures were transferred to fresh 250 mL glass bottom-baffled flasks and 2% (v/v) oleic acid and 2% (v/v) glycerol were added. Cultures were incubated at 30° C. with shaking at 300 rpm. Samples were taken at 120 hours for GC-FID and GC-MS analysis. Control strain sAA2382 produced 1.55 g/L hexanoic acid but did not produce any olivetolic acid. All other strains produced less than 0.03 g/L hexanoic acid and produced olivetolic acid with improved production with addition of glycerol.

A second shake flask experiment was conducted with strains sVZ0074 and sVZ0206 with modifications. Growth stage flasks inoculated to an initial OD_{600 nm} of 0.4 were incubated at 30° C. with shaking at 250 rpm. After centrifugation and resuspension in HiP-TAB media the cultures were transferred to 250 mL glass bottom-baffled flasks and 4% (v/v) oleic acid and 2% (v/v) glycerol were added. Cultures were incubated at 30° C. with shaking at 250 rpm. Glycerol was supplemented every 24 hours and cultures were harvested after 72 hours. Yeast cells from 2 mL of whole broth were collected by centrifugation, washed twice in 1× phosphate buffered saline (PBS), and lysed by bead-beating in 2 mL of 1×PBS. Lysate samples were analyzed for olivetolic acid by Infinite Chemical Analysis (San Diego). Results are shown in table below.

TABLE 4
Olivetolic Acid in Cell Lysates of Engineered Strains

| Strain | Olivetolic acid (mg/L) |
|---|---|
| SAA0074 | 13.1 |
| SAA0206 | 3.0 |

### Example 21: Production of Cannabigerolic Acid from Oleic Acid with Glycerol Supplementation

Strains containing an engineered beta-oxidation pathway and an engineered pathway for the production of cannabigerolic acid were investigated for their ability to produce cannabigerolic acid in shake flask testing with slight modifications to the basic protocol (Example 14, above). 250 mL glass flasks containing 50 mL of rich media (yeast nitrogen base, 6.7 g/L; yeast extract, 3.0 g/L; ammonium sulfate, 3.0 g/L; potassium phosphate monobasic, 1.0 g/L; potassium phosphate dibasic, 1.0 g/L; glycerol, 75 g/L) were inoculated with a 5 mL YPD overnight culture to an initial OD_{600 nm} of 0.4. After 24 h incubation at 30° C. with shaking at 250 rpm, the cells were centrifuged and the cell pellet resuspended in 15 mL of HiP-TAB media (yeast nitrogen base without amino acids and without ammonium sulfate, 1.7 g/L; yeast extract, 3.0 g/L; potassium phosphate monobasic, 10.0 g/L; potassium phosphate dibasic, 10.0 g/L). The cultures were transferred to fresh 250 mL glass bottom-baffled flasks and 4% (v/v) oleic acid and 2% (v/v) glycerol were added. Cultures were incubated at 30° C. with shaking at 300 rpm. Oleic acid was added again to 4% (v/v) after 72 hours. Glycerol was added again to 2% (v/v) after 24, 72, and 120 hours. Cultures were harvested after 168 hours. Yeast cells from 2 mL of whole broth were collected by centrifugation, washed twice in 1× phosphate buffered saline (PBS), and lysed by bead-beating in 1×PBS. Cell-free supernatant and lysate samples were analyzed for olivetolic acid and cannabigerolic acid by Infinite Chemical Analysis (San Diego). Results are shown in table below.

TABLE 5
Cannabinoids in Supernatants and Cell
Lysates of Engineered Strains

| | Olivetolic acid (mg/L) | | Cannabigerolic acid (mg/L) | |
|---|---|---|---|---|
| Strain | Supernatant | Lysate | Supernatant | Lysate |
| sRB008 | 4.14 | 12.23 | ND | ND |
| sRB010 | 5.86 | 11.04 | 0.67 | 1.51 |
| sRB018 | 1.33 | 8.96 | ND | ND |

### Example 22: Production of Cannabidiolic Acid from Fatty Acids

Strains containing an engineered beta-oxidation pathway and an engineered pathway for the production of cannabidiolic acid such as those in Example 15, may be investigated for their ability to produce cannabidiolic acid in shake flask testing by using a carbon source feeding strategy similar to that performed in Examples 19 through 21. Samples from both cell lysates and cell-free supernatant may be analyzed for the presence of cannabidiolic acid inside or secreted from the cells, respectively.

### BIBLIOGRAPHY

Fellermeier, M and Zenk, M. H. (1998) FEBS Letters 427: 283-285.
Gagne, S. J., Stout, J. M., Liu, E., Boubakir, Z., Clark, S. M., and Page, J. E. (2012) PNAS 109: 12811-12816.
Hatanaka, A (1999), In: Sankawa U, ed. “Comprehensive natural products chemistry, vol. 1”, Oxford: Elsevier, 83-115.
Marks, M. D., Tian, L., Wenger, J. P., Omburo, S. N., Soto-Fuentes, W., He, J., Gang, D. R., Weiblen, G. D., and Dixon, R. A. (2009) J Exp Botany 60: 3715-3726.
Morimoto, S., Komatsu, K., Taura, F., and Shoyama, Y. (1998) Phytochemistry 49: 1525-1529.
Page, J. E. and Boubakir, Z (2014) U.S. Pat. No. 8,884,100.
Sirikantaramas, S., Morimoto, S., Shoyama, Yo., Ishikawa, Y., Wada, Y., Shoyama, Yu., and Taura, F. (2004) JBC 279: 39767-39774.
Sirikantaramas, S., Taura, F., Tanaka, Y., Ishikawa, Y., Morimoto, S., and Shoyama, Y. (2005) Plant Cell Physiol 46: 1578-1582.
Stout, J. M., Boubakir, Z., Ambrose, S. J., Purves, R. W., and Page, J. E. (2012) Plant J 71: 353-365.
Taura, F., Morimoto, S., and Shoyama, Y. (1995) JACS 117: 9766-9767.
Taura, F., Morimoto, S., and Shoyama, Y. (1996) JBC 271: 17411-17416.
Taura, F., Sirikantaramas, S., Shoyama, Yo., Yoshikai, K., Shoyama, Yu., and Morimoto, S. (2007) FEBS Letters 581: 2929-2934.
Taura, F., Tanaka, S., Taguchi, C., Fukamizu, T., Tanaka, H., Shoyama, Y., and Morimoto, S. (2009) FEBS Letters 583: 2061-2066.
Zirpel, B., Degenhardt, F., Martin, C., Oliver, K., and Stehle, F. (2017) J Biotechnology 259: 204-212.

All of the compositions and methods described and claimed herein can be made and executed without undue experimentation in light of the present disclosure. While the compositions and methods of this invention have been described in terms of preferred embodiments, it will be apparent to those of skill in the art that variations may be applied to the compositions and methods. All such similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Sequences: Oligos

| Oligo | SEQ ID | Oligo Sequence |
|---|---|---|
| Name | NO: | |
| oAA2206 | 36 | TTCCGCTTAATGGAGTCCAAA |
| oAA2209 | 37 | TAAACGTTGGGCAACCTTGG |
| oAA3458 | 38 | CACACAGCTCTTCAGCCATGACAGAAGTTGTTGATAG |
| oAA3491 | 39 | ATGACAGAAGTTGTTGATAGAGCCTCATC |
| oAA3492 | 40 | TCTTGACTTACCGGCCTTGATG |
| oAA3750 | 41 | CACACAGCTCTTCGAGCCTACAACTTGGCTCTTGACTTACCGGCCTTGA |
| oAA4394 | 42 | TAGGTTAATTAAA |
| oAA4395 | 43 | AGCTTTTAATTAACC |
| oAA4966 | 44 | CTCTGGTTCTGGTGTCTTTC |
| oAA4967 | 45 | GCCGGCATAACATATAAGTC |
| oAA5908 | 46 | GATTGATTGTTATAGTTTCTTTCTTTC |
| oAA6054 | 47 | GAGTGACTCTTTTGATAAGAGTC |
| oAA7433 | 48 | TGACCCCCTATCGCTACGGT |
| oAA7434 | 49 | ATGGGGAGGAGGACGAGGAA |
| oAA7682 | 50 | CCACGTCGGTACCGAGATCGTTGCCGAGGCAATCAAGTCCTT |
| oAA7683 | 51 | AACGGCTTCGTCTAAACAACCACGGATCTTCAACAATCCCTGTTCTGGAC |
| oAA7684 | 52 | GTCCAGAACAGGGATTGTTGAAGATCCGTGGTTGTTTAGACGAAGCCGTT |
| oAA7685 | 53 | AGTGTTTGTGTCCGGTAACGACCGAAATATTACAATTGGAGCTCC |
| oAA7686 | 54 | GGAGCTCCAATTGTAATATTTCGGTCGTTACCGGACACAAACACT |
| oAA7687 | 55 | TTTCAGCAACGGCATCACC |
| oAA7941 | 56 | ACCTTTATGCCAACATCAGACC |
| oAA7942 | 57 | AACGGCTTCGTCTAAACAACCCATCAACGGTGTACTTTTCAGTATCC |
| oAA7943 | 58 | GGATACTGAAAAGTACACCGTTGATGGGTTGTTTAGACGAAGCCGTT |
| oAA7944 | 59 | TTGCAATGCCATGAACGCCCGAAATATTACAATTGGAGCTCC |
| oAA7945 | 60 | GGAGCTCCAATTGTAATATTTCGGGCGTTCATGGCATTGCAA |
| oAA7946 | 61 | CAGATGGCAACAATCCCAAG |
| oAA9863 | 62 | ATCGATTAAATTCTTTAATTGAGGGATGTG |
| oAA9864 | 63 | GAGTGACTCTTTTGATAAGAGTCGCAAATTTGATTTCA |
| oAA9865 | 64 | CAATTAAAGAATTTAATCGATATGGGTTTGTCCTCTGTG |
| oAA9866 | 65 | CCCTTCATCTTTATCGTGATAATCAACCCAAAG |
| oAA9867 | 66 | ATCACGATAAAGATGAAGGGCGGTCCAT |
| oAA9868 | 67 | TCTTATCAAAAGAGTCACTCTCAGATAAACACGTAAACCAAATATTC |
| oAA9869 | 68 | CAATTAAAGAATTTAATCGATATGAACCCTAGAGAAAACTTC |
| oAA9870 | 69 | TAATAGTATCAATCCATGACAACTGTCG |
| oAA9871 | 70 | GTCATGGATTGATACTATTATATTTTACAGTGGAGTG |
| oAA9872 | 71 | TCTTATCAAAAGAGTCACTCTCAGTGTCTATGCCTAGG |
| oVZ0151 | 72 | GATTGATTGTTATAGTTTCTTTCTTTCTTTTGAGGATGACCAGATG |
| oVZ0152 | 73 | GAGTGACTCTTTTGATAAGAGTCG |
| oVZ0153 | 74 | AAGAAAGAAACTATAACAATCAATCATGAACCATTTGAGGGCTGAAG |
| oVZ0154 | 75 | GCGACTCTTATCAAAAGAGTCACTCTTAATACTTGATAGGAACGCTTCTG |
| oVZ0155 | 76 | AAGAAAGAAACTATAACAATCAATCATGGCCGTTAAACACTTGATAG |
| oVZ0156 | 77 | GCGACTCTTATCAAAAGAGTCACTCTTATTTCCGCGGCGTATAATC |
| oVZ0337 | 78 | AATTAACCTATGGTGCAC |
| oVZ0338 | 79 | TTAATTAAAAGCTTGGCGTAATC |
| oVZ0339 | 80 | CTCGTGCTAGTCAGTCTTGCACGCTTTGGGTG |
| oVZ0340 | 81 | ATGATTACGCCAAGCTTTTAATTAACAACACGGCGTCTGAGGAC |
| oVZ0341 | 82 | ACTGAGAGTGCACCATAGGTTAATTAACTCGGGGCCGTCGGTGGA |
| oVZ0342 | 83 | AAGCGTGCAAGACTGACTAGCACGAGCGAAGATGGGG |
| oVZ0369 | 84 | GTCTTGCACGCTTTGGGTG |
| oVZ0370 | 85 | TGACTAGCACGAGCGAAG |
| oVZ0371 | 86 | TCCCCATCTTCGCTCGTGCTAGTCAAAGGGAAGAAGAGTCGTTG |
| oVZ0372 | 87 | CGTCGGCACCCAAAGCGTGCAAGACGTCGACCTAAATTCGCAAC |
| oVZ0373 | 88 | CTCGGGGCCGTCGGTGGA |
| oVZ0374 | 89 | CAACACGGCGTCTGAGGACTTGG |
| oVZ0941 | 90 | AGAAACTATAACAATCAATCATGACCACTTTGCCTTCGATC |
| oVZ0942 | 91 | TCTTATCAAAAGAGTCACTCTCACAACTTGTAATCTTTGACAATG |
| oVZ0968 | 92 | GAAACTATAACAATCAATCATGGGCAAGAACTACAAGAG |
| oVZ0969 | 93 | TTATCAAAAGAGTCACTCTCATTCGAAATGACTAAATTG |
| oVZ1117 | 94 | TGAGAGTGACTCTTTTGATAA |
| oVZ1118 | 95 | ATCTTTGACAATGGAGCCT |
| oRB0001 | 96 | GAATAGAAGAGAGTGACTCTTTTGATAAGAGTCG |
| oRB0002 | 97 | GATTGATTGTTATAGTTTCTTTCTTTCT |
| oRB0008 | 98 | GCAATTTGGGACTCCTTCATG |
| oRB0009 | 99 | AACCAACCAGTAAGCTTGCA |
| oRB0010 | 100 | CTCGGGGCCGTCGGTGGA |
| oRB0011 | 101 | CAACACGGCGTCTGAGGACTTGG |

| gBlock | Sequence |
|---|---|
| bRB001 | tgttgcaagcttactggttggttgccgatgatatgatgga |
| (SEQ ID | ccaatccaagaccagaagaggacagaaatgttggtacttg |
| NO: 14) | gtcgaaggtgttggaaacattgcaatttgggactccttca |
| | tgttg |

Sequences: Plasmids

| Gene symbol | Gene name | Synonyms | Pathway | EC # | Organism | Oligos |
|---|---|---|---|---|---|---|
| AuACO1^{+pts} | acyl-CoA | N/A | beta- | 1.3.3.6 | Arthrobacter | N/A |
| | oxidase | | oxidation | | ureafaciens | |

| | | |
|---|---|---|
| Gene symbol | plasmid | Note |
| AuACO1^{+pts} | pAA964/pAA965 | Codon-Optimized for C. viswanathii |

| Gene symbol | Gene Sequence (start to stop) |
|---|---|
| AuACO1^{+pts} | atgacagaagttgttgatagagcctcatcaccagcctcaccaggttcaacaacagccgccgccgatggtgccaaggttgccgttg |
| (SEQ ID | aaccaagagttgatgttgccgccttgggtgaacaattgttgggtagatgggccgacatcagattgcacgccagagatttggccgg |
| NO: 1) | tagagaagttgttcaaaaggttgaaggtttgacacacacagaacacagatcaagagattcggtcaattgaagtacttggttgata |
| | acaacgccgttcacagagccttcccatcaagattgggtggttcagatgatcacggtggtaacatcgccggtttcgaagaattggt |
| | tacagccgatccatcattgcaaatcaaggccggtgttcaatggggtttgttcggttcagccgttatgcacttgggtacaagagaa |
| | caccacgataagtggttgccaggtatcatgtcattggaaatcccaggttgatcgccatgacagaaacaggtcacggttcagatga |
| | gcctcaatcgccacaacagccacatacgatgaagaaacacaagagttcgttatcgatacaccattcagagccgcctggaaggatt |
| | acatcggtaacgccgccaacgatggtttggccgccgttgttttcgcccaattgatcacaagaaaggttaaccacggtgttcacgc |
| | cttctacgttgatttgcgcgatccagccacaggtgatttcttgccaggtatcggtggtgaagatgatggtatcaagggtggtttg |
| | aacggtatcgataacggtagattgcacttcacaaacgttagaatcccaagaacaaacttgttgaacagatacggtgatgagccgt |
| | tgatggtacatactcatcaacaatcgaatcaccaggtagaagattcttcacaatgagggtacattggttcaaggtagagtttcat |
| | tggatggtgccgccgttgccgcctcaaaggttgccttgcaatcagccatccactacgccgccgaaagaagacaattcaacgccac |
| | atcaccaacagaagaagaagattgaggattaccaaagacaccaaagaagattgttcacaagattggccacaacatacgccgcctc |
| | attcgcccacgaacaattgagcaaaagttcgatgatgattctcaggtgcccacgatacagatgccgatagacaagatttggaaac |
| | attggccgccgccttgaagccattgtcaacatggcacgccttggatacattgcaagaatgtagagaagcctgtggtggtgccggt |
| | ttcttgatcgaaaacagattcgcctcattgagagccgatttggatgtttacgttacattcgaaggtgataacacagttttgttgc |
| | aattggttgccaagagattgttggccgattacgccaaggagttcagaggtgccaacttcggtgttttggccagatacgttgttga |
| | tcaagccgccggtgttgccttgcacagaacaggtttgagacaagttgcccaattcgttgccgattcaggttcagttcaaaagtca |
| | gccttggccttgagagatgaagaaggtcaaagaacattgttgacagatagagttcaatcaatggttgccgaagttggtgccgcct |
| | tgaagggtgccggtaagttgccacaacaccaagctgccgccttgttcaaccaacaccaaaacgaattgatcgaagccgcccaagc |
| | ccacgccgaattgagcaatgggaagccttcacagaagccaggccaaggttgatgatgccggtacaaaggaagattgacaagattg |
| | agagatttgttcggtttgtcattgatcgaaaagcacttgtcatggtacttgatgaacggtagattgtcaatgcaaagaggtagaa |
| | cagttggtacatacatcaacagattgttggttaagatcagaccacacgccttggatttggttgatgccttcggttacggtgccga |
| | acacttgagagccgccatcgccacaggtgccgaagccacaagacaagatgaagccagaacatacttcagacaacaaagagcctca |
| | ggttcagccccagccgatgaaaagacattgttggccatcaaggccggtaagtcaagagccaagttgtag |

| Gene symbol | Protein Sequence |
|---|---|
| AuACO1^{+pts} | MTEVVDRASSPASPGSTTAAADGAKVAVEPRVDVAALGEQLLGRWADIRLHARDLA |
| (SEQ ID NO: 19) | GREVVQKVEGLTHTEHRSRVFGQLKYLVDNNAVHRAFPSRLGGSDDHGGNIAGFEEL |
| | VTADPSLQIKAGVQWGLFGSAVMHLGTREHHDKWLPGIMSLEIPGCFAMTETGHGSD |
| | VASIATTATYDEETQEFVIDTPFRAAWKDYIGNAANDGLAAVVFAQLITRKVNHGVH |
| | AFYVDLRDPATGDFLPGIGGEDDGIKGGLNGIDNGRLHFTNVRIPRTNLLNRYGDVAV |
| | DGTYSSTIESPGRRFFTMLGTLVQGRVSLDGAAVAASKVALQSAIHYAAERRQFNATS |
| | PTEEEVLLDYQRHQRRLFTRLATTYAASFAHEQLLQKFDDVFSGAHDTDADRQDLET |
| | LAAALKPLSTWHALDTLQECREACGGAGFLIENRFASLRADLDVYVTFEGDNTVLLQ |
| | LVAKRLLADYAKEFRGANFGVLARYVVDQAAGVALHRTGLRQVAQFVADSGSVQKS |
| | ALALRDEEGQRTLLTDRVQSMVAEVGAALKGAGKLPQHQAAALFNQHQNELIEAAQ |
| | AHAELLQWEAFTEALAKVDDAGTKEVLTRLRDLFGLSLIEKHLSWYLMNGRLSMQR |
| | GRTVGTYINRLLVKIRPHALDLVDAFGYGAEHLRAAIATGAEATRQDEARTYFRQQR |
| | ASGSAPADEKTLLAIKAGKSRAKL |

| Gene symbol | Gene name | Synonyms | Pathway | EC # | Organism | Oligos |
|---|---|---|---|---|---|---|
| PKS-6xHis | olivetolic | tetraketide | cannabinoid | 2.3.1.206 | Cannabis | N/A |
| | acid | synthase | synthesis | | sativa | |
| | synthase | | | | | |
| | with 6xHis | | | | | |
| | tag | | | | | |

| | | |
|---|---|---|
| Gene symbol | plasmid | Note |
| PKS-6xHis | pVZ3970 | Codon-Optimized for C. viswanathii |

| Gene symbol | Gene Sequence (start to stop) |
|---|---|
| PKS-6xHis | atgaaccatttgagggctgaagggccagcttctgtgttggccataggtacggctaacccagaaaacattcttcttcaggat |
| (SEQ ID NO: | gaatttccagattactattttagagtgaccaaatctgagcacatgactcaactcaaggaaaagtttagaaagatctgtgac |
| 2) | aaatcaatgataaggaagcgtaattgtttccttaacgaagaacaccttaagcaaaaccctcgattggttgagcacgaaatg |
| | cagacattagatgcacgacaagatatgttggttgttgaagtgccaaagcttggtaaggacgcttgtgccaaggccataaag |
| | gaatggggtcaacctaaaagtaaaattacccacttgatattcacgtcagccagcacgacagacatgccaggggcagactac |
| | cattgtgcaaaattgttaggtagtcaccgtcggtcaaacgcgttatgatgtaccaattaggctgctatgggggaggaaccg |
| | tcttgaggattgctaaggacattgccgaaaacaacaagggtgcacgagtgcttgctgtctgagcgacatcatggcttgcct |
| | ctttagaggtccaagcgagagtgatttggaattactcgtcggccaagcgatcttcggtgacggcgccgctgcggtgattgt |
| | aggtgctgaaccagacgaatcggtgggtgaacgtccaattacgagaggtgagcacgggtcaaacaatattgcctaattctg |
| | aagggaccatcggcggacatatcagagaggcagggttaatattgatttgcacaaggacgttcccatgagatcagcaacaat |
| | atcgagaaatgtttgatcgaggcctttacaccgattggtatctcggattggaactccatcttctggattactcatccagga |
| | ggaaaggccatcttggataaggttgaggagaagttgcacttgaaatcagataagttcgtcgatagtcgtcatgtccttagt |
| | gaacacggcaacatgtcgtcgtccacggttcattcgtcatggacgaactcaggaaaaggtccaggaggaagggaagtcaac |
| | gacaggagacgggtttgagtggggtgtattgtttggttttggacccggtttgaccgtcgaacgtgttgtggtcagaagcgt |
| | tcctatcaagtatcatcatcaccatcaccactaa |

| Gene symbol | Protein Sequence |
|---|---|
| PKS-6xHis | MNHLRAEGPASVLAIGTANPENILLQDEFPDYYFRVTKSEHMTQLKEKFRKIC |
| (SEQ ID NO: | DKSMIRKRNCFLNEEHLKQNPRLVEHEMQTLDARQDMLVVEVPKLGKDAC |
| 20) | AKAIKEWGQPKSKITHLIFTSASTTDMPGADYHCAKLLGLSPSVKRVMMYQL |
| | GCYGGGTVLRIAKDIAENNKGARVLAVCCDIMACLFRGPSESDLELLVGQAIF |
| | GDGAAAVIVGAEPDESVGERPIFELVSTGQTILPNSEGTIGGHIREAGLIFDLHK |
| | DVPMLISNNIEKCLIEAFTPIGISDWNSIFVVITHPGGKAILDKVEEKLHLKSDKF |
| | VDSRHVLSEHGNMSSSTVLFVMDELRKRSLEEGKSTTGDGFEWGVLFGFGPG |
| | LTVERVVVRSVPIKYHHHHHH |

| Gene symbol | Gene name | Synonyms | Pathway | EC # | Organism | Oligos |
|---|---|---|---|---|---|---|
| OAC-6xHis | olivetolic | N/A | cannabinoid | 4.4.1.26 | Cannabis | N/A |
| | acid cyclase | | synthesis | | sativa | |
| | with 6xHis | | | | | |
| | tag | | | | | |

| | | |
|---|---|---|
| Gene symbol | plasmid | Note |
| OAC-6xHis | pVZ3968 | Codon-Optimized for C. viswanathii |

| Gene symbol | Gene Sequence (start to stop) |
|---|---|
| OAC-6xHis | atggccgttaaacacttgatagtgttgaagtttaaggatgaaataacggaagctcaaaaagaagagtttttcaagactt |
| (SEQ ID NO: | atgtcaatttagttaacattatcccagcgatgaaagatgtgtactggggtaaagacgtgacccagaagaacaaagaaga |
| 3) | gggttatacacatattgtggaggttacgttcgagtcagtggaaacgatccaggattacattatccatccagcccacgtc |
| | ggattcggggatgatacagatcattctgggaaaaacattgatcacgattatacgccgcggaaacatcatcaccatcacc |
| | actaa |

| Gene symbol | Protein Sequence |
|---|---|
| OAC-6xHis | MAVKHLIVLKFKDEITEAQKEEFFKTYVNLVNIIPAMKDVYWGKDVTQKNK |
| (SEQ ID NO: | EEGYTHIVEVTFESVETIQDYIIHPAHVGFGDVYRSFVVEKLLIFDYTPRKHHHH |
| 21) | HH |

| Gene symbol | Gene name | Synonyms | Pathway | EC# | Organism | Oligos |
|---|---|---|---|---|---|---|
| PKS | olivetolic | tetraketide | cannabinoid | 2.3.1.206 | Cannabis | N/A |
| | acid synthase | synthase | synthesis | | sativa | |

| Gene symbol | plasmid | Note |
|---|---|---|
| PKS | pVZ4009 | Codon-Optimized for C. viswanathii |

| Gene symbol | Gene Sequence (start to stop) |
|---|---|
| PKS | atgaaccatttgagggctgaagggccagcttctgtgttggccataggtacggctaacccagaaaacattcttcttcaggatg |
| (SEQ ID NO: | aatttccagattactattttagagtgaccaaatctgagcacatgactcaactcaaggaaaagtttagaaagatctgtgacaa |
| 4) | atcaatgataaggaagcgtaattgtttccttaacgaagaacaccttaagcaaaaccctcgattggttgagcacgaaatgcag |
| | acattagatgcacgacaagatatgttggttgttgaagtgccaaagcttggtaaggacgcttgtgccaaggccataaaggaat |
| | ggggtcaacctaaaagtaaaattacccacttgatattcacgtcagccagcacgacagacatgccaggggcagactaccattg |
| | tgcaaaattgttaggtttgtcaccgtcggtcaaacgcgttatgatgtaccaattaggctgctatgggggaggaaccgtcttg |
| | aggattgctaaggacattgccgaaaacaacaagggtgcacgagtgcttgctgtctgagcgacatcatggcttgcctctttag |
| | aggtccaagcgagagtgatttggaattactcgtcggccaagcgatcttcggtgacggcgccgctgcggtgattgtaggtgct |
| | gaaccagacgaatcggtgggtgaacgtccaattacgagaggtgagcacgggtcaaacaatattgcctaattctgaagggacc |
| | atcggcggacatatcagagaggcagggttaatattgatttgcacaaggacgttcccatgagatcagcaacaatatcgagaa |
| | atgtttgatcgaggcctttacaccgattggtatctcggattggaactccatcttctggattactcatccaggaggaaaggcc |
| | atcttggataaggttgaggagaagttgcacttgaaatcagataagttcgtcgatagtcgtcatgtccttagtgaacacggca |
| | acatgtcgtcgtccacggttcattcgtcatggacgaactcaggaaaaggtccaggaggaagggaagtcaacgacaggagacg |
| | ggtttgagtggggtgtattgtttggttttggacccggtttgaccgtcgaacgtgttgtggtcagaagcgttcctatcaagta |
| | ttaa |

| Gene symbol | Protein Sequence |
|---|---|
| PKS | MNHLRAEGPASVLAIGTANPENILLQDEFPDYYFRVTKSEHMTQLKEKFRKIC |
| (SEQ ID NO: | DKSMIRKRNCFLNEEHLKQNPRLVEHEMQTLDARQDMLVVEVPKLGKDAC |
| 22) | AKAIKEWGQPKSKITHLIFTSASTTDMPGADYHCAKLLGLSPSVKRVMMYQL |
| | GCYGGGTVLRIAKDIAENNKGARVLAVCCDIMACLFRGPSESDLELLVGQAIF |
| | GDGAAAVIVGAEPDESVGERPIFELVSTGQTILPNSEGTIGGHIREAGLIFDLHK |
| | DVPMLISNNIEKCLIEAFTPIGISDWNSIFVVITHPGGKAILDKVEEKLHLKSDKF |
| | VDSRHVLSEHGNMSSSTVLFVMDELRKRSLEEGKSTTGDGFEWGVLFGFGPG |
| | LTVERVVVRSVPIKY |

| Gene symbol | Gene name | Synonyms | Pathway | EC# | Organism | Oligos |
|---|---|---|---|---|---|---|
| OAC | olivetolic | N/A | cannabinoid | 4.4.1.26 | Cannabis | N/A |
| | acid cyclase | | synthesis | | sativa | |

| Gene symbol | plasmid | Note |
|---|---|---|
| OAC | pVZ4008 | Codon-Optimized for C. viswanathii |

| Gene symbol | Gene Sequence (start to stop) |
|---|---|
| OAC | atggccgttaaacacttgatagtgagaagtttaaggatgaaataacggaagctcaaaaagaagagtattcaagacttatgt |
| (SEQ ID NO: | caatttagttaacattatcccagcgatgaaagatgtgtactggggtaaagacgtgacccagaagaacaaagaagagggtta |
| 5) | tacacatattgtggaggttacgttcgagtcagtggaaacgatccaggattacattatccatccagcccacgtcggattcgg |
| | ggatgtttacagatcattctgggaaaaacattgatcacgattatacgccgcggaaataa |

| Gene symbol | Protein Sequence |
|---|---|
| OAC | MAVKHLIVLKFKDEITEAQKEEFFKTYVNLVNIIPAMKDVYWGKDVTQKNKE |
| (SEQ ID NO: | EGYTHIVEVTFESVETIQDYIIHPAHVGFGDVYRSFVVEKLLIFDYTPRK |
| 23) | |

| Gene symbol | Gene name | Synonyms | Pathway | EC# | Organism | Oligos |
|---|---|---|---|---|---|---|
| CBDAS (no | cannabidiolic acid | N/A | cannabinoid | 1.21.3.8 | Cannabis | N/A |
| signal peptide) | synthase | | synthesis | | sativa | |

| Gene symbol | plasmid | Note |
|---|---|---|
| CBDAS (no | pAA3632, pRB0075 | Codon-Optimized for C. viswanathii |
| signal peptide) | | |

| Gene symbol | Gene Sequence (start to stop) |
|---|---|
| CBDAS (no | atgaaccctagagaaaacttcctcaagtgtttttcccaatatatcccaaacaatgctacaaatcttaaattggtctatacccagaa |
| signal peptide) | caatcccttatacatgagtgtccttaactcaacaatccataatctccgtttcacatcggacacgactccaaaaccgttggtaatag |
| (SEQ ID NO: 8) | tgactccgtcccacgtcagtcacattcagggcactatcctctgaccaagaaggtcggtttgcaaattagaacaagatcgggcgggc |
| | acgatagcgaggggatgtcgtacatctctcaagttccattcgttatcgttgacttgcgaaatatgcgctccattaagattgatgtt |
| | catagccaaaccgcgtgggttgaagcaggcgctactcttggggaggtctactattgggtgaacgaaaaaaatgaaaacttgtcact |
| | cgctgcaggctactgcccaacggtgtgtgctggcggacattttggtggtggggggtatggtcccctcatgcgaaattacgggttag |
| | ccgcagataatataatagacgctcacctcgtgaacgtccacggtaaagttctagatcggaagtccatgggtgaagatttgactggg |
| | ctcttcggggaggtggtgcagagtcattggaatcatcgttgcgtggaaaattaggttggtggctgtccccaagtctacgatgtttt |
| | ctgttaaaaagatcatggagatccatgagctagttaagttagttaataagtggcaaaacattgcctataagtatgacaaggatttg |
| | cttctaatgacgcacttcatcactagaaacatcaccgacaaccaaggcaaaaacaagaccgcaatacacacttacttcagctcagt |
| | atttttaggaggggtggattcattggttgacttgatgaataaatcgttcccagaattgggtatcaagaaaaccgattgtcgacagt |
| | tgtcatggattgatactattatattttacagtggagtggttaactacgacacagacaactttaacaaggagattttgttggacaga |
| | tctgccgggcagaacggggcctttaaaataaagcttgattatgttaaaaagcccatcccagagagcgtgttcgtccaaatcttgga |
| | aaagttgtacgaagaggatatcggcgcaggcatgtacgctttgtacccctacggtggtattatggatgaaatctcggagtccgcaa |
| | ttccttttccccatagagccggtattttgtacgagttgtggtacatttgttcgtgggagaaacaagaggacaacgaaaagcatttg |
| | aactggatacggaacatttataacttcatgacaccatacgttagtaagaacccgaggaggcttacttaaactatcgagacctcgac |
| | attgggatcaacgatccgaaaaacccaaacaactacacacaagcccgcatctggggagaaaagtactttggaaagaacttcgatag |
| | attggtcaaggtgaagacacttgtggacccaaacaacttcttcagaaacgagcagtctattccacctttgcctaggcatagacact |
| | ga |

| Gene symbol | Protein Sequence |
|---|---|
| CBDAS (no | MNPRENFLKCFSQYIPNNATNLKLVYTQNNPLYMSVLNSTIHNLRFTSDTTPKPLVIVTPS |
| signal peptide) | HVSHIQGTILCSKKVGLQIRTRSGGHDSEGMSYISQVPFVIVDLRNMRSIKIDVHSQTAWV |
| (SEQ ID NO: 26) | EAGATLGEVYYWVNEKNENLSLAAGYCPTVCAGGHFGGGGYGPLMRNYGLAADNIID |
| | AHLVNVHGKVLDRKSMGEDLFVVALRGGGAESFGIIVAWKIRLVAVPKSTMFSVKKIMEI |
| | HELVKLVNKWQNIAYKYDKDLLLMTHFITRNITDNQGKNKTAIHTYFSSVFLGGVDSLV |
| | DLMNKSFPELGIKKTDCRQLSWIDTIIFYSGVVNYDTDNFNKEILLDRSAGQNGAFKIKL |
| | DYVKKPIPESVFVQILEKLYEEDIGAGMYALYPYGGIMDEISESAIPFPHRAGILYELWYIC |
| | SWEKQEDNEKHLNWIRNIYNFMTPYVSKNPRLAYLNYRDLDIGINDPKNPNNYTQARIVV |
| | GEKYFGKNFDRLVKVKTLVDPNNFFRNEQSIPPLPRHRH |

| Gene symbol | Gene name | Synonyms | Pathway | EC# | Organism | Oligos |
|---|---|---|---|---|---|---|
| CBDAS | cannabidiolic acid | N/A | cannabinoid | 1.21.3.8 | Cannabis | N/A |
| | synthase with signal | | synthesis | | sativa | |
| | sequence | | | | | |

| Gene symbol | plasmid | Note |
|---|---|---|
| CBDAS | pVZ4124 | Codon-Optimized for C. viswanathii |

| Gene symbol | Gene Sequence (start to stop) |
|---|---|
| CBDAS | atgaagtgctcgacattctctttctggttcgtttgtaagattatcttctttttttttagtttcaacatccagacctcaattgctaa |
| (SEQ ID | ccctagagaaaacttcctcaagtgtttttcccaatatatcccaaacaatgctacaaatcttaaattggtctatacccagaacaatc |
| NO: 9) | ccttatacatgagtgtccttaactcaacaatccataatctccgtttcacatcggacacgactccaaaaccgttggtaatagtgact |
| | ccgtcccacgtcagtcacattcagggcactatcctctgttccaagaaggtcggtttgcaaattagaacaagatcgggcgggcacga |
| | tagcgaggggatgtcgtacatctctcaagttccattcgttatcgttgacttgcgaaatatgcgctccattaagattgatgttcata |
| | gccaaaccgcgtgggttgaagcaggcgctactcttggggaggtctactattgggtgaacgaaaaaaatgaaaacttgtcactcgct |
| | gcaggctactgcccaacggtgtgtgctggcggacattttggtggtggggggtatggtcccctcatgcgaaattacgggttagccgc |
| | agataatataatagacgctcacctcgtgaacgtccacggtaaagttctagatcggaagtccatgggtgaagatttgttctgggctc |
| | ttcggggaggtggtgcagagtcctttggaatcatcgttgcgtggaaaattaggttggtggctgtccccaagtctacgatgttttct |
| | gttaaaaagatcatggagatccatgagctagttaagttagttaataagtggcaaaacattgcctataagtatgacaaggatttgct |
| | tctaatgacgcacttcatcactagaaacatcaccgacaaccaaggcaaaaacaagaccgcaatacacacttacttcagctcagtat |
| | ttttaggaggggtggattcattggttgacttgatgaataaatcgttcccagaattgggtatcaagaaaaccgattgtcgacagagt |
| | catggattgatactattatattttacagtggagtggttaactacgacacagacaactttaacaaggagattttgttggacagatct |
| | gccgggcagaacggggcctttaaaataaagcttgattatgttaaaaagcccatcccagagagcgtgttcgtccaaatcttggaaaa |
| | gttgtacgaagaggatatcggcgcaggcatgtacgctttgtacccctacggtggtattatggatgaaatctcggagtccgcaattc |
| | cttttccccatagagccggtattagtacgagttgtggtacatttgacgtgggagaaacaagaggacaacgaaaagcatttgaactg |
| | gatacggaacatttataacttcatgacaccatacgttagtaagaacccgaggttggcttacttaaactatcgagacctcgacattg |
| | ggatcaacgatccgaaaaacccaaacaactacacacaagcccgcatctggggagaaaagtactttggaaagaacttcgatagattg |
| | gtcaaggtgaagacacttgtggacccaaacaacacttcagaaacgagcagtctattccacctttgcctaggcatagacactga |

| Gene symbol | Protein Sequence |
|---|---|
| CBDAS | MKCSTFSFVVFVCKIIFFFFSFNIQTSIANPRENFLKCFSQYIPNNATNLKLVYTQNNPLYMS |
| (SEQ ID NO: 27) | VLNSTIHNLRFTSDTTPKPLVIVTPSHVSHIQGTILCSKKVGLQIRTRSGGHDSEGMSYISQ |
| | VPFVIVDLRNMRSIKIDVHSQTAWVEAGATLGEVYYWVNEKNENLSLAAGYCPTVCAG |
| | GHFGGGGYGPLMRNYGLAADNIIDAHLVNVHGKVLDRKSMGEDLFVVALRGGGAESFG |
| | IIVAWKIRLVAVPKSTMFSVKKIMEIHELVKLVNKWQNIAYKYDKDLLLMTHFITRNITD |
| | NQGKNKTAIHTYFSSVFLGGVDSLVDLMNKSFPELGIKKTDCRQLSWIDTIIFYSGVVNY |
| | DTDNFNKEILLDRSAGQNGAFKIKLDYVKKPIPESVFVQILEKLYEEDIGAGMYALYPYG |
| | GIMDEISESAIPFPHRAGILYELWYICSWEKQEDNEKHLNVVIRNIYNFMTPYVSKNPRLA |
| | YLNYRDLDIGINDPKNPNNYTQARIVVGEKYFGKNFDRLVKVKTLVDPNNFFRNEQSIPPL |
| | PRHRH |

| Gene symbol | Gene name | Synonyms | Pathway | EC# | Organism | Oligos |
|---|---|---|---|---|---|---|
| AAE1 | fatty acyl-CoA | N/A | cannabinoid | 6.2.1.2 | Cannabis | N/A |
| | synthetase | | synthesis | | sativa | |

| Gene symbol | plasmid | Note |
|---|---|---|
| AAE1 | pVZ4282 | Codon-Optimized for C. viswanathii |

| Gene symbol | Gene Sequence (start to stop) |
|---|---|
| AAE1 | atgggcaagaactacaagagtttagactcggttgtggctagtgactttatcgctctcggcatcacgtcggaagtggctgagacct |
| (SEQ ID | tgcacggtaggttggccgaaattgtgtgcaattatggagctgctacaccacaaacctggattaacatcgcaaatcatattctatc |
| NO: 10) | ccctgacttgccttttagtttgcaccagatgttgttctatggttgctataaggatttcggcccagcaccaccagcgtggattccc |
| | gacccagaaaaagtcaaatcaacgaacttaggtgcgttgttggagaaaagagggaaggaattcttgggagtgaagtataaagatc |
| | ctattagctcattctctcactacaagaattaccgttagaaacccagaggtatattggcgaacagtgttgatggatgagatgaaga |
| | tttctttctcaaaggacccagaatgcattctaagacgagacgatataaacaacccgggcggatctgaatggttgcccggcggata |
| | tctaaactcggcaaagaactgcttaaatgtgaactcaaacaagaagttgaacgatactatgatagtttggcgggacgaaggaaac |
| | gacgacttgcccctaaataaattaacgttggaccaattgagaaagcgagtttggcttgttgggtatgcgctagaagaaatgggat |
| | tggaaaaggggtgtgctattgcaattgatatgccaatgcatgttgacgcggtggtcatttacttggcaatcgtgctagctggata |
| | cgtggtcgttagtatagctgactctttttcagccccagaaatcagcacgaggttgcgcttgtcaaaggctaaggccattttcacg |
| | caagaccacatcatcagaggcaagaagcgtataccgttgtactctcgtgttgtcgaggccaaatcccctatggccatcgtcattc |
| | catgctcaggtagtaatattggtgctgagctccgagatggagacatatcttgggattatttcttggaaagagcaaaagagttcaa |
| | aaactgcgagttcactgcgagggagcaaccggtcgatgcgtacactaacatattatttagttcgggcaccacgggcgagccaaaa |
| | gcaattccgtggacgcaggctactccactaaaggctgcagctgatggctggtcgcacttggacattagaaagggtgatgtgattg |
| | tgtggccaacgaacttgggttggatgatggggccatggttagtctacgcgtctagttgaatggtgcctcgatcgcgctatacaat |
| | ggtagtccattggtctctgggttcgccaaattcgtccaagacgccaaggttacgatgttgggggtcgttccgtccattgtgagat |
| | cgtggaagagtacaaactgtgtttcaggatatgattggtccaccatcagatgtttctcaagctcaggcgaagcgtcgaatgtcga |
| | tgagtacttgtggctcatggggcgtgctaactataaaccagttattgagatgtgtggtggcaccgaaatcggtggtgccacagtg |
| | caggttctttcttacaggcgcagagtttgagttctttctcatcccaatgcatggggtgcacattgtatatcttggacaagaatgg |
| | gtatccgatgccaaagaacaaaccgggcataggagagttggccctcggtcccgtgatgttcggggctagcaagactttgttgaac |
| | gggaaccaccatgatgtgtacttcaagggaatgccaacattgaatggggaggtcttgagacgtcacggggatatcttcgaattaa |
| | cctccaacggttattaccacgctcatggtagagcagatgacacaatgaacattggtggcatcaagatatcatcaatagagataga |
| | aagggtatgtaacgaggttgacgatagagatttgaaacaactgcaattggtgttccgcccttgggtggcggtccagaacagcttg |
| | ttattttcttcgttttgaaagactcaaacgatactacaatcgatcttaatcaattgagattatcgttcaacctaggcttgcagaa |
| | gaagttaaatcccttgttcaaggtaacaagggttgttccgttgtcgtcattgccacgcactgctactaacaagataatgagaaga |
| | gttttgcgacaacaatttagtcatttcgaatga |

| Gene symbol | Protein Sequence |
|---|---|
| AAE1 | MGKNYKSLDSVVASDFIALGITSEVAETLHGRLAEIVCNYGAATPQTWINIANHI |
| (SEQ ID | LSPDLPFSLHQMLFYGCYKDFGPAPPAWIPDPEKVKSTNLGALLEKRGKEFLGV |
| NO: 28) | KYKDPISSFSHFQEFSVRNPEVYWRTVLMDEMKISFSKDPECILRRDDINNPGGSE |
| | WLPGGYLNSAKNCLNVNSNKKLNDTMIVWRDEGNDDLPLNKLTLDQLRKRVW |
| | LVGYALEEMGLEKGCAIAIDMPMHVDAVVIYLAIVLAGYVVVSIADSFSAPEIST |
| | RLRLSKAKAIFTQDHIIRGKKRIPLYSRVVEAKSPMAIVIPCSGSNIGAELRDGDIS |
| | WDYFLERAKEFKNCEFTAREQPVDAYTNILFSSGTTGEPKAIPWTQATPLKAAA |
| | DGWSHLDIRKGDVIVWPTNLGWMMGPWLVYASLLNGASIALYNGSPLVSGFA |
| | KFVQDAKVTMLGVVPSIVRSWKSTNCVSGYDWSTIRCFSSSGEASNVDEYLWL |
| | MGRANYKPVIEMCGGTEIGGAFSAGSFLQAQSLSSFSSQCMGCTLYILDKNGYP |
| | MPKNKPGIGELALGPVMFGASKTLLNGNHHDVYFKGMPTLNGEVLRRHGDIFE |
| | LTSNGYYHAHGRADDTMNIGGIKISSIEIERVCNEVDDRVFETTAIGVPPLGGGPE |
| | QLVIFFVLKDSNDTTIDLNQLRLSFNLGLQKKLNPLFKVTRVVPLSSLPRTATNKI |
| | MRRVLRQQFSHFE |

| Gene symbol | Gene name | Synonyms | Pathway | EC# | Organism | Oligos |
|---|---|---|---|---|---|---|
| ACS2d | fatty acyl-CoA | N/A | beta-oxidation | 6.2.1.2 | Candida | N/A |
| | synthetase | | | | viswanathii | |

| Gene symbol | plasmid | Note |
|---|---|---|
| ACS2d | pVZ4285 | N/A |

| Gene symbol | Gene Sequence (start to stop) |
|---|---|
| ACS2d | atgaccactttgccttcgatcgcagacaccgacaacatctatgcctccgacgataaggagtacattttcgacaaccccaacgacttgtccattgaga |
| (SEQ ID NO: 11) | ctctcgtcaaccacatccttccatttcctcaggaagttgctggtgaatctgtcaaggttcctggcactgctgtccagggtttctctgaattctacagaaat |
| | gctgctactcccaacgggatcaagttgagtttgatcaaggggttggacacttatcatcatatatttgagagctctgctgagcgttacgctgacgaccc |
| | gtgccttgcgttccacgagtacgactacgagaactcgcagcatttagagcgatacgcaaccatctcctacaaggaagtgcgccaaagaaaggatg |
| | attttgccgctggtttgttctttctcttgaaggctaacccttacaagaacgattccttggaggcacatcaaaaaatcgtcaaccatgaagccaactacaa |
| | gctgtacgacagcgacaacatgtccttcattgtcacgttctacgccgcaaatagagtcgagtgggtcttgtctgacttggcgtgctcctccaattccat |
| | tacatcgacggcattgtacgacacgttgggcccagatacgtccaagtatattttggagactaccgaatctcctgtcattattagctccaaggaccatat |
| | tcgcgacttgattgacttgaagaaggcaaaccccaaggaacttgctgctcttatcttgattatttcgatggacccgttgaagaaatctgaccagaactt |
| | ggttcatttggcagaagcaaacaatatcaagttgtatgatttctcccaagtcgaaagaactggagctattttcccccatcaaaccaatgccccaaaca |
| | gcgaaactgtcttcacaatcaccttcacttcaggaactactggtgccaatccaaaaggtgttgtccttcctcaacgatgtgctgcctcgggtatgagg |
| | cgtatagtgttatgatgcctcaccacaggggcacgagggagtttgcgttcttgccattagcacacatttttgagagacagatggttgcttcgatgatat |
| | gtttggtggctcgtccgcaatgccacgattgggcggtacgccattgaccttggtagaagacttgaagttgtggaaacctacgttcatggccaatgttc |
| | cacgtgattcaccaagattgaagcgggcatcaaagcttcgacgatcgactccacttccagcctcacgagatcattgtatgaacgtgctatcgaagc |
| | aaagcgtgtcaagcagaacaagaacgacgacagtggagaccactttatctatgacaagttgttgattcaaagattgagaagcgctatcgggtatga |
| | ctgtttggaattctgtgtcactggtagtgctccaattgctcctgaaactatcaaattcttgaaagctagtttgggaattggatttggtcaaggatatggta |
| | gcagtgaatcgtttgctggaatgttgtttgctttgcctttcaagaactccagtgtcggaacctgtggcgtcatctcgcccactatggaggccagattaa |
| | gagagttaccagacatgggttacatgttgaacgataagaatggaccacgaggagagttgcaactccgtggttctcaattattcaccaggtactacaa |
| | gaatccagaagaaactgcaaagtccatcgatgaagacggttggttcagtaccggtgacgttgctgagattggcacagatggctatttcagaattatt |
| | gacagggtgaagaacttctacaaattatcccagggtgagtatgtttcgccagagaagattgagagtagtacttgtcgttgaactcgagtattctgcag |
| | ctttttatccatggggactctaccaagtcatttctcgtgggtgtggtgggtttacagcctgatgttgccagcaagtatgttgatctttcttccggtcccaat |
| | gtggtccaagtgttaaaccaacctgagtttagaaagcaattattgttagacctaaacctgaaggtcaatggcaaattgcaagggtttgaaaagttgca |
| | caacattttcatcgacattgaaccattgacactcgagagaaatgttgttaccccaacaatgaagctcaagagacattttgctgccaagtttttcaagcc |
| | ccagatcgaagctatgtatgcagaaggctccattgtcaaagattacaagttgtga |

| Gene symbol | Protein Sequence |
|---|---|
| ACS2d | MTTLPSIADTDNIYASDDKEYIFDNPNDLSIETLVNHILPFPQEVAGESVKVPGTAVQGFSEFY |
| (SEQ ID NO: | RNAATPNGIKLSLIKGLDTYHHIFESSAERYADDPCLAFHEYDYENSQHLERYATISYKEVRQ |
| 29) | RKDDFAAGLFFLLKANPYKNDSLEAHQKIVNHEANYKSYDSDNMSFIVTFYAANRVEWVLS |
| | DLACSSNSITSTALYDTLGPDTSKYILETTESPVIISSKDHIRDLIDLKKANPKELAALILIISMD |
| | PLKKSDQNLVHLAEANNIKLYDFSQVERTGAIFPHQTNAPNSETVFTITFTSGTTGANPKGVV |
| | LPQRCAASGMLAYSVMMPHHRGTREFAFLPLAHIFERQMVASMFMFGGSSAMPRLGGTPL |
| | TLVEDLKLWKPTFMANVPRVFTKIEAGIKASTIDSTSSLTRSLYERAIEAKRVKQNKNDDSG |
| | DHFIYDKLLIQRLRSAIGYDCLEFCVTGSAPIAPETIKFLKASLGIGFGQGYGSSESFAGMLFA |
| | LPFKNSSVGTCGVISPTMEARLRELPDMGYMLNDKNGPRGELQLRGSQLFTRYYKNPEETA |
| | KSIDEDGWFSTGDVAEIGTDGYFRIIDRVKNFYKLSQGEYVSPEKIESLYLSLNSSISQLFIHGD |
| | STKSFLVGVVGLQPDVASKYVDLSSGPNVVQVLNQPEFRKQLLLDLNSKVNGKLQGFEKLH |
| | NIFIDIEPLTLERNVVTPTMKLKRHFAAKFFKPQIEAMYAEGSIVKDYKL* |

| Gene symbol | Gene name | Synonyms | Pathway | EC# | Organism | Oligos |
|---|---|---|---|---|---|---|
| ACS2d^{Δpts} | fatty acyl-CoA | N/A | beta-oxidation | 6.2.1.2 | Candida | N/A |
| | synthetase (deleted | | | | viswanathii | |
| | PTS1) | | | | | |

| Gene symbol | plasmid | Note |
|---|---|---|
| ACS2d^{Δpts} | pVZ4348 | N/A |

| Gene symbol | Gene Sequence (start to stop) |
|---|---|
| ACS2d^{Δpts} | atgaccactttgccttcgatcgcagacaccgacaacatctatgcctccgacgataaggagtacattttcgacaaccccaacgacttgtccattgagactct |
| (SEQ ID NO: 12) | cgtcaaccacatccaccatttcctcaggaagagctggtgaatctgtcaaggacctggcactgctgtccagggatctctgaattctacagaaatgctgct |
| | actcccaacgggatcaagttgagtttgatcaaggggttggacacttatcatcatatatttgagagctctgctgagcgttacgctgacgacccgtgccttgc |
| | gttccacgagtacgactacgagaactcgcagcatttagagcgatacgcaaccatctcctacaaggaagtgcgccaaagaaaggatgattttgccgctg |
| | gtttgactttctcttgaaggctaacccttacaagaacgattccttggaggcacatcaaaaaatcgtcaaccatgaagccaactacaagctgtacgacagc |
| | gacaacatgtccttcattgtcacgttctacgccgcaaatagagtcgagtgggtcttgtctgacttggcgtgctcctccaattccattacatcgacggcattgt |
| | acgacacgttgggcccagatacgtccaagtatattttggagactaccgaatctcctgtcattattagctccaaggaccatattcgcgacttgattgacttga |
| | agaaggcaaaccccaaggaacttgctgctcttatcttgattatttcgatggacccgttgaagaaatctgaccagaacttggttcatttggcagaagcaaac |
| | aatatcaagttgtatgatttctcccaagtcgaaagaactggagctattttcccccatcaaaccaatgccccaaacagcgaaactgtcttcacaatcaccttc |
| | acttcaggaactactggtgccaatccaaaaggtgttgtccttcctcaacgatgtgctgcctcgggtatgttggcgtatagtgttatgatgcctcaccacagg |
| | ggcacgagggagtagcgttcttgccattagcacacatattgagagacagatggttgcttcgatgatatgatggtggctcgtccgcaatgccacgattg |
| | ggcggtacgccattgaccttggtagaagacttgaagagtggaaacctacgttcatggccaatgaccacgtgattcaccaagattgaagcgggcatca |
| | aagcttcgacgatcgactccacttccagcctcacgagatcattgtatgaacgtgctatcgaagcaaagcgtgtcaagcagaacaagaacgacgacagt |
| | ggagaccactttatctatgacaagttgttgattcaaagattgagaagcgctatcgggtatgactgtttggaattctgtgtcactggtagtgctccaattgctc |
| | ctgaaactatcaaattcttgaaagctagtttgggaattggatttggtcaaggatatggtagcagtgaatcgtttgctggaatgttgtttgctttgcctttcaaga |
| | actccagtgtcggaacctgtggcgtcatctcgcccactatggaggccagattaagagagttaccagacatgggttacatgttgaacgataagaatggac |
| | cacgaggagagttgcaactccgtggactcaattattcaccaggtactacaagaatccagaagaaactgcaaagtccatcgatgaagacggttggttca |
| | gtaccggtgacgttgctgagattggcacagatggctatttcagaattattgacagggtgaagaacttctacaaattatcccagggtgagtatgtttcgcca |
| | gagaagattgagagtttgtacttgtcgttgaactcgagtattctgcagctattatccatggggactctaccaagtcatttctcgtgggtgtggtgggataca |
| | gcctgatgttgccagcaagtatgttgatctttcttccggtcccaatgtggtccaagtgttaaaccaacctgagtttagaaagcaattattgttagacctaaac |
| | ctgaaggtcaatggcaaattgcaagggatgaaaagttgcacaacatatcatcgacattgaaccattgacactcgagagaaatgttgttaccccaacaat |
| | gaagctcaagagacattttgctgccaagtattcaagccccagatcgaagctatgtatgcagaaggctccattgtcaaagattga |

| Gene symbol | Gene Sequence (start to stop) |
|---|---|
| ACS2d^{Δpts} | MTTLPSIADTDNIYASDDKEYIFDNPNDLSIETLVNHILPFPQEVAGESVKVPGTAVQGFSEFYRN |
| (SEQ ID NO: | AATPNGIKLSLIKGLDTYHHIFESSAERYADDPCLAFHEYDYENSQHLERYATISYKEVRQRKD |
| 30) | DFAAGLFFLLKANPYKNDSLEAHQKIVNHEANYKSYDSDNMSFIVTFYAANRVEWVLSDLACS |
| | SNSITSTALYDTLGPDTSKYILETTESPVIISSKDHIRDLIDLKKANPKELAALILIISMDPLKKSDQ |
| | NLVHLAEANNIKLYDFSQVERTGAIFPHQTNAPNSETVFTITFTSGTTGANPKGVVLPQRCAASG |
| | MLAYSVMMPHHRGTREFAFLPLAHIFERQMVASMFMFGGSSAMPRLGGTPLTLVEDLKLWKP |
| | TFMANVPRVFTKIEAGIKASTIDSTSSLTRSLYERAIEAKRVKQNKNDDSGDHFIYDKLLIQRLRS |
| | AIGYDCLEFCVTGSAPIAPETIKFLKASLGIGFGQGYGSSESFAGMLFALPFKNSSVGTCGVISPT |
| | MEARLRELPDMGYMLNDKNGPRGELQLRGSQLFTRYYKNPEETAKSIDEDGWFSTGDVAEIGT |
| | DGYFRIIDRVKNFYKLSQGEYVSPEKIESLYLSLNSSISQLFIHGDSTKSFLVGVVGLQPDVASKY |
| | VDLSSGPNVVQVLNQPEFRKQLLLDLNSKVNGKLQGFEKLHNIFIDIEPLTLERNVVTPTMKLK |
| | RHFAAKFFKPQIEAMYAEGSIVKD* |

| Gene symbol | Gene name | Synonyms | Pathway | EC# | Organism | Oligos |
|---|---|---|---|---|---|---|
| CvERG20 | Farnesyl diphosphate | N/A | Isoprenoid | 2.5.1.1, | Candida | N/A |
| | synthase | | | 2.5.1.10 | viswanathii | |

| Gene symbol | plasmid | Note |
|---|---|---|
| CvERG20 | pVZ4105 | N/A |

| Gene symbol | Gene Sequence (start to stop) |
|---|---|
| (CvERG20 | atgtctgataaagcagccgctagagagagattcctctctgtttttgagtgtgccgtcgaggaattgaaagaagtcttggtt |
| SEQ ID NO: | tctcacaagatgccgcaagaagcaattgactggtttgtcaagaacttgaactacaacacccccggcggtaagttgaacaga |
| 13) | ggtagtctgagtcgacacctacgctatcttgaacaacaccaccgctgacaagttgaacgatgaacaatacaagaaggtcgc |
| | cttgagggctggtcaattgaattgagcaagcttactattggttgccgatgatatgatggaccaatccaagaccagaagagg |
| | acagaaatgttggtacttggtcgaaggtgttggaaacattgcaattaatgactccttcatgttggaaggtgccatttacgt |
| | cttgttgaagaagcatcttccgtcaagatccatactatgtcgacttgaggacttgaccacgaagtcaccaccagaccgaat |
| | tgggcaattattggacttggtgactgctgatgaagaagtcgtcgacttggacaagactccttggacaagcactcgttcatt |
| | gtcattttcaaaaccgcatactactccactacttgcctgagctttggccatgtacatgagcggtatcagcagcgaagaaga |
| | cttgaagcaagtcagagatatcttgatcccattgggtgagtacttccaaatccaggacgatttcttggactgtacggaacc |
| | ccagaacaaattggcaagatcggtactgatatcaaagacaacaagtgttcctgggtggtcaaccaagctagttgcatgcta |
| | ctccagaacaacgtaagttgttggacgacaactacggtaagaaagacgacgagtctgaacagagatgcaaggacttgttca |
| | agtccatgggcattgaaaagatctaccacgactacgaagagtcaattgttgctaaattaagagaacaaatcgataaagttg |
| | atgaatcaagaggtttgaaaaaagatgtcttgaccgctttcttgggcaaggtttacaagagatccaaatag |

| Gene symbol | Gene Sequence (start to stop) |
|---|---|
| (CvERG20 | MSDKAAARERFLSVFECAVEELKEVLVSHKMPQEAIDWFVKNLNYNTPGGKLNRGLSVVDT |
| SEQ ID NO: | YAILNNTTADKLNDEQYKKVALLGWSIELLQAYFLVADDMMDQSKTRRGQKCWYLVEGVG |
| 31) | NIAINDSFMLEGAIYVLLKKHFRQDPYYVDLLDLFHEVTFQTELGQLLDLVTADEEVVDLDK |
| | FSLDKHSFIVIFKTAYYSFYLPVALAMYMSGISSEEDLKQVRDILIPLGEYFQIQDDFLDCFGTP |
| | EQIGKIGTDIKDNKCSWVVNQALLHATPEQRKLLDDNYGKKDDESEQRCKDLFKSMGIEKIY |
| | HDYEESIVAKLREQIDKVDESRGLKKDVLTAFLGKVYKRSK* |

| Gene symbol | Gene name | Synonyms | Pathway | EC# | Organism | Oligos |
|---|---|---|---|---|---|---|
| CvERG20 | Farnesyl | N/A | Isoprenoid | 2.5.1.1, | Candida | N/A |
| (F95W, N126W) | diphosphate | | | 2.5.1.10 | viswanathii | |
| | synthase | | | | | |

| Gene symbol | plasmid | Note |
|---|---|---|
| CvERG20 | pRB0076 | N/A |
| (F95W, N126W) | | |

| Gene symbol | Gene Sequence (start to stop) |
|---|---|
| CvERG20 | atgtctgataaagcagccgctagagagagattcctctctgtttttgagtgtgccgtcgaggaattgaaagaagtcttggtttc |
| (F95W, N126W) | tcacaagatgccgcaagaagcaattgactggtttgtcaagaacttgaactacaacacccccggcggtaagttgaacagaggta |
| (SEQ ID NO: | gtctgagtcgacacctacgctatcttgaacaacaccactgctgacaagttgaacgatgaacaatacaagaaggtcgccttgag |
| 15) | ggctggtcaattgaattgagcaagcttactggaggttgccgatgatatgatggaccaatccaagaccagaagaggacagaaat |
| | gttggtacttggtcgaaggtgttggaaacattgcaatttgggactccttcatgttggaaggtgccatttacgtcttgttgaag |
| | aagcacttccgtcaagatccatactatgtcgacttgttggacttgttccacgaagtcaccttccagaccgaattgggtcaatt |
| | attggacttggtgactgctgatgaagaagtcgtcgacttggacaagactccttggacaagcactcgttcattgtcattttcaa |
| | aaccgcatactactccttctacttgcctgagctttggccatgtacatgagcggtatcagcagcgaagaagacttgaagcaagt |
| | cagagatatcttgatcccattgggtgagtacttccaaatccaggacgatttcttggactgtttcggaaccccagaacaaattg |
| | gcaagatcggtactgatatcaaagacaacaagtgttcctgggtggtcaaccaagctttgttgcatgctactccagaacaacgt |
| | aagttgttggacgacaactacggtaagaaagacgacgagtctgaacagagatgcaaggacttgttcaagtccatgggcattga |
| | aaagatctaccacgactacgaagagtcaattgttgctaaattaagagaacaaatcgataaagttgatgaatcaagaggtttga |
| | aaaaagatgtcttgaccgctttcttgggcaaggtttacaagagatccaaatag |

| Gene symbol | Gene Sequence (start to stop) |
|---|---|
| CvERG20 | MSDKAAARERFLSVFECAVEELKEVLVSHKMPQEAIDWFVKNLNYNTPGGKLNRGLSVVDT |
| (F95W, N126W) | YAILNNTTADKLNDEQYKKVALLGWSIELLQAYWLVADDMMDQSKTRRGQKCWYLVEGV |
| (SEQ ID NO: | GNIAIWDSFMLEGAIYVLLKKHFRQDPYYVDLLDLFHEVTFQTELGQLLDLVTADEEVVDLD |
| 32) | KFSLDKHSFIVIFKTAYYSFYLPVALAMYMSGISSEEDLKQVRDILIPLGEYFQIQDDFLDCFGT |
| | PEQIGKIGTDIKDNKCSWVVNQALLHATPEQRKLLDDNYGKKDDESEQRCKDLFKSMGIEKI |
| | YHDYEESIVAKLREQIDKVDESRGLKKDVLTAFLGKVYKRSK* |

| Gene symbol | Gene name | Synonyms | Pathway | EC# | Organism | Oligos |
|---|---|---|---|---|---|---|
| CsPT1 | geranylpyrophosphate- | GOT | cannabinoid | 2.5.1.102 | Cannabis | N/A |
| | olivetolic acid | | synthesis | | sativa | |
| | transferase | | | | | |

| Gene symbol | plasmid | Note |
|---|---|---|
| CsPT1 | pAA3636, pRB0074 | Codon optimized for Candida viswanathii |

| Gene symbol | Gene Sequence (start to stop) |
|---|---|
| CsPT1 | atgggtagtcctctgtgtgtacattcagcttccaaacgaactatcatacgttgttaaacccacacaacaataaccccaagacttc |
| (SEQ ID NO: | cttgctatgctaccgacacccaaaaacccccataaagtattcttacaacaatttcccctcaaagcactgttcgacaaagagcttc |
| 6) | cacttgcagaataagtgctccgaaagatgtctatcgccaagaactcaatcagggctgcaacaaccaaccaaaccgaacctccaga |
| | atcggataaccactccgttgctacgaaaatcttgaactaggtaaggcatgaggaagttgcaacgaccgtacacaattatcgcctt |
| | ccacctcatgcgcctgtggtctatcggtaaggaattactacataacacaaactaatcagctggtcattaatgataaagcgttatc |
| | ttcctcgtagccatcttgtgtattgctagattaccactactattaatcaaatctacgacttgcacattgatagaatcaacaaacc |
| | agatctaccatggcatccggagaaattagtgttaatacagcatggatcatgagcattatcgtggcactctttgggttgattatca |
| | cgataaagatgaagggcggtccattgtacatattcgggtactgatcggaatattggagggattgatacagcgttccaccatttcg |
| | ttggaaacaaaatccgagtacagcattcctattgaacttcaggcacacatcataacaaatttcacattctactacgcttcccggg |
| | ccgccagggtctcccgtttgagttgagacctagtttcacatttcttttggctttcatgaagtctatgggttcggcattggcccta |
| | atcaaggatgcctctgatgtcgaaggtgatacaaagttcggaatttcaacgctcgcatctaagtacgggagccgaaacttgaccc |
| | ttttctgctctgggattgtgttgttgtcgtatgtagccgcaatcttggcaggaattatatggccccaagcgtttaattccaatgt |
| | tatgcttattctcatgctatattggccactggttgattctccaaactagggattttgccctcacaaattacgatccagaagccgg |
| | aagacgatttatgaatttatgtggaagttgtactacgctgaatatttggatacgtgatatctga |

| Gene symbol | Gene Sequence (start to stop) |
|---|---|
| CsPT1 | MGLSSVCTFSFQTNYHTLLNPHNNNPKTSLLCYRHPKTPIKYSYNNFPSKHCSTKSFHLQNKC |
| (SEQ ID NO: | SESLSIAKNSIRAATTNQTEPPESDNHSVATKILNFGKACWKLQRPYTIIAFTSCACGLFGKELL |
| 24) | HNTNLISWSLMFKAFFFLVAILCIASFTTTINQIYDLHIDRINKPDLPLASGEISVNTAWIMSIIV |
| | ALFGLIITIKMKGGPLYIFGYCFGIFGGIVYSVPPFRWKQNPSTAFLLNFLAHIITNFTFYYASRA |
| | ALGLPFELRPSFTFLLAFMKSMGSALALIKDASDVEGDTKFGISTLASKYGSRNLTLFCSGIVL |
| | LSYVAAILAGIIVVPQAFNSNVMLLSHAILAFVVLILQTRDFALTNYDPEAGRRFYEFMWKLYY |
| | AEYLVYVFI |

| Gene symbol | Gene name | Synonyms | Pathway | EC# | Organism | Oligos |
|---|---|---|---|---|---|---|
| CsPT1 (no | geranylpyrophosphate- | GOT | cannabinoid | 2.5.1.102 | Cannabis | N/A |
| signal peptide) | olivetolic acid | | synthesis | | sativa | |
| | transferase | | | | | |

| Gene symbol | plasmid | Note |
|---|---|---|
| CsPT1 (no | pRB0073 | Codon optimized for Candida viswanathii |
| signal peptide) | | |

| Gene symbol | Gene Sequence (start to stop) |
|---|---|
| CsPT1 (no | atggctgcaacaaccaaccaaaccgaacctccagaatcggataaccactccgttgctacgaaaatcttgaactttggtaag |
| signal peptide) | gcatgttggaagttgcaacgaccgtacacaattatcgccttcacctcatgcgcctgtggtcttttcggtaaggaattacta |
| (SEQ ID NO: | cataacacaaacctaatcagctggtcattaatgataaagcgttcttcttcctcgtagccatcttgtgtattgctagtttta |
| 7) | ccactactattaatcaaatctacgacttgcacattgatagaatcaacaaaccagatctacctttggcatccggagaaatta |
| | gtgttaatacagcatggatcatgagcattatcgtggcactctttgggttgattatcacgataaagatgaagggcggtccat |
| | tgtacatattcgggtactgtttcggaatctttggagggattgtttacagcgttccaccatttcgttggaaacaaaatccga |
| | gtacagcattcctattgaacttcttggcacacatcataacaaatttcacattctactacgcttcccgggccgccttgggtc |
| | tcccgtttgagttgagacctagtttcacatttcttttggctacatgaagtctatgggacggcattggccctaatcaaggat |
| | gcctctgatgtcgaaggtgatacaaagttcggaatttcaacgctcgcatctaagtacgggagccgaaacttgacccttttc |
| | tgctctgggattgtgttgttgtcgtatgtagccgcaatcttggcaggaattatatggccccaagcgtttaattccaatgtt |
| | atgcttctactcatgctatattggccactggttgattctccaaactagggattttgccctcacaaattacgatccagaagc |
| | cggaagacgtattatgaatttatgtggaagttgtactacgctgaatatttggtttacgtgtttatctga |

| Gene symbol | Gene Sequence (start to stop) |
|---|---|
| CsPT1 (no | MAATTNQTEPPESDNHSVATKILNFGKACWKLQRPYTIIAFTSCACGLFGKELLHNTNLISWS |
| signal peptide) | LMFKAFFFLVAILCIASFTTTINQIYDLHIDRINKPDLPLASGEISVNTAWIMSIIVALFGLIITIKM |
| (SEQ ID NO: | KGGPLYIFGYCFGIFGGIVYSVPPFRWKQNPSTAFLLNFLAHIITNFTFYYASRAALGLPFELRP |
| 25) | SFTFLLAFMKSMGSALALIKDASDVEGDTKFGISTLASKYGSRNLTLFCSGIVLLSYVAAILAG |
| | IIVVPQAFNSNVMLLSHAILAFVVLILQTRDFALTNYDPEAGRRFYEFMWKLYYAEYLVYVFI* |

| Gene symbol | Gene name | Synonyms | Pathway | EC# | Organism | Oligos |
|---|---|---|---|---|---|---|
| CsPT4 (no | geranylpyrophosphate- | GOT | cannabinoid | 2.5.1.102 | Cannabis | N/A |
| signal peptide) | olivetolic acid | | synthesis | | sativa | |
| | transferase | | | | | |

| Gene symbol | plasmid | Note |
|---|---|---|
| CsPT4 (no | pRB0077 | Codon optimized for Candida viswanathii |
| signal peptide) | | |

| Gene symbol | Gene Sequence (start to stop) |
|---|---|
| CsPT4 (no | atggctggcagcgaccaaatcgaaggctcgccacaccacgaatcggacaactccattgctaccaagatccttaacttcggt |
| signal peptide) | cacacctgctggaagttacaaagaccttacgttgttaaggggatgatctccatagcctgcggcttgttcgggagagagttg |
| (SEQ ID NO: | ttcaacaatagacaccttttctcttggggcttgatgtggaaagccttcttcgccttggtaccaatcttgagtttcaacttc |
| 16) | ttcgctgctatcatgaatcaaatctatgacgtagacatagatcgtatcaacaaaccagacctacctctagtctccggggaa |
| | atgtccatcgaaactgcctggatcttgagcatcatcgtagctctaaccgggttgattgdaccatcaagttgaagtcggctc |
| | ctttgttcgtcttcatctatatcttcggcatcttcgccgggttcgcgtactcggtccctcctatcagatggaaacaatacc |
| | cattcacaaacttcctaatcacgattagactcacgtaggacttgcctttacttcctactcggctacaacttccgccttagg |
| | ccttcctttcgtatggcgtccagccttctcgttcataattgccttcatgactgtcatgggcatgaccatcgccttcgcgaa |
| | ggacatctccgacatcgaaggagatgcgaagtacggggtttccaccgtcgctacaaagcttggcgcccggaacatgacttt |
| | cgtcgtctcgggtgtccttcttctcaactacttggtctcgatttcgatcgggatcatctggccacaggtcttcaaatcgaa |
| | catcatgattctttcgcacgctatacttgctttctgtctcatcdtcagacccgggagttagccctagctaactatgcgtca |
| | gcacctagcagacagttctttgagttcatatggttgctctactacgccgaatacttcgtttacgttttcatttga |

| Gene symbol | Gene Sequence (start to stop) |
|---|---|
| CsPT4 (no | MAGSDQIEGSPHHESDNSIATKILNFGHTCWKLQRPYVVKGMISIACGLFGRELFNNRHLFSW |
| signal peptide) | GLMWKAFFALVPILSFNFFAAIMNQIYDVDIDRINKPDLPLVSGEMSIETAWILSIIVALTGLIV |
| (SEQ ID NO: | TIKLKSAPLFVFIYIFGIFAGFAYSVPPIRWKQYPFTNFLITISSHVGLAFTSYSATTSALGLPFV |
| 33) | WRPAFSFIIAFMTVMGMTIAFAKDISDIEGDAKYGVSTVATKLGARNMTFVVSGVLLLNYLV |
| | SISIGIIVVPQVFKSNIMILSHAILAFCLIFQTRELALANYASAPSRQFFEFIVVLLYYAEYFVYVFI* |

| Gene symbol | Gene name | Synonyms | Pathway | EC# | Organism | Oligos |
|---|---|---|---|---|---|---|
| NphB(G286S, Y288A) | aromatic prenyl | GOT | cannabinoid | 2.5.1.102 | Streptomyces sp. | N/A |
| | transferase | | synthesis | | | |

| Gene symbol | plasmid | Note |
|---|---|---|
| NphB(G286S, Y288A) | pRB0085 | Codon optimized for Candida viswanathii |

| Gene symbol | Gene Sequence (start to stop) |
|---|---|
| NphB (G286S, | atgagtgaagctgccgatgttgagcgagtttacgccgctatggaggaagctgcaggacttttaggggtagcctgtgctcgtgat |
| Y288A) | aagatttaccctttattatcaaccttccaagacacattagtggagggtgggtctgtagttgttttctcaatggctagtggacga |
| (SEQ ID NO: | cactctactgagttggatttcagtatatccgtgcctacatcacacggggacccatacgccactgtagtagagaaaggacttttt |
| 17) | cctgcaacaggacacccagttgatgatcttcttgccgatacccagaaacaccttcctgtatccatgtttgctatcgacggggag |
| | gtgacaggtgggtttaagaagacttatgccttttttccaaccgacaatatgcctggagagctgagcttagtgctatcccatcca |
| | tgccacctgctgtggctgagaacgccgagcttttcgctcgttacggtcttgataaggttcaaatgacctcaatggactacaaga |
| | agcgtcaggtgaacctttacttctcagagctttcagcccaaacattggaggcagagagtgtattggcccttgttagagagttag |
| | gacttcacgtaccaaatgagttaggtttgaaattttgcaaaagatccttctccgtttaccctacattaaactgggagaccggta |
| | agattgatcgtttgtgctttgctgttatcagtaacgacccaactttggtgccttcaagtgatgagggtgatatcgagaagtttc |
| | acaattatgcaacaaaagctccttatgcctacgttggggagaagcgaactaggtttatggtcttactctttccccaaaggaaga |
| | atattataagctttcagccgcttatcatataactgatgtgcaacgagggcacttaaggcttttgatagtttggaagactaa |

| Gene symbol | Gene Sequence (start to stop) |
|---|---|
| NphB (G286S, | MSEAADVERVYAAMEEAAGLLGVACARDKIYPLLSTFQDTLVEGGSVVVFSMASGRHSTEL |
| Y288A) | DFSISVPTSHGDPYATVVEKGLFPATGHPVDDLLADTQKHLPVSMFAIDGEVTGGFKKTYAFF |
| (SEQ ID NO: | PTDNMPGVAELSAIPSMPPAVAENAELFARYGLDKVQMTSMDYKKRQVNLYFSELSAQTLE |
| 34) | AESVLALVRELGLHVPNELGLKFCKRSFSVYPTLNVVETGKIDRLCFAVISNDPTLVPSSDEGDI |
| | EKFHNYATKAPYAYVGEKRTLVYGLTLSPKEEYYKLSAAYHITDVQRGLLKAFDSLED* |

| Gene symbol | Gene name | Synonyms | Pathway | EC# | Organism | Oligos |
|---|---|---|---|---|---|---|
| CBCAS (no | Cannabichromenic | N/A | cannabinoid | 1.21.3.- | Cannabis | N/A |
| signal peptide) | acid synthase | | synthesis | | sativa | |

| Gene symbol | plasmid | Note |
|---|---|---|
| CBCAS (no | pRB0084 | Codon optimized for Candida viswanathii |
| signal peptide) | | |

| Gene symbol | Gene Sequence (start to stop) |
|---|---|
| CBCAS (no | atgaaccctcaggaaaattttttaaagtgcttttccgagtatatcccaaataaccctgctaatcctaagttcatttacactcaacac |
| signal | gatcagttgtatatgtcagattgaactccacaattcaaaacttaagattcacttccgatactaccccaaagcctttggttatagtaa |
| peptide) | caccttccaatgtctctcacatccaggcttcaatactttgctccaaaaaggtagggcttcagatacgtactagatctggaggacacg |
| (SEQ ID NO: | acgcagaaggattgtcttatatatcacaggttccatttgctatcgtagacttgagaaacatgcacacagttaaggttgatattcact |
| 18) | cacaaactgcatgggtggaggctggtgctactttgggtgaggtatactattggattaacgaaatgaatgaaaacttctccttccctg |
| | gaggatattgtcctactgtaggggtaggtgggcatttctctggtggggggtatggggcattgatgcgaaactatggtttggctgccg |
| | acaatataatagatgcacaccttgtgaatgtagatgggaaagttttagatcgaaagtccatgggggaggatttattctgggcaatac |
| | gagggggagggggtgaaaacttcgggatcatcgcagcctgcaagatcaagttggttgtcgtcccatctaaggctacaatattctcag |
| | tgaagaagaatatggagatacatggtcttgtaaaactttttaataaatggcagaatatcgcatacaagtacgacaaggatttaatgt |
| | tgacaacccattttcgtactcgtaatattactgataatcatgggaaaaataagactacagtccacgggtatttcagtagtatattct |
| | taggaggtgttgacagtttagttgacttgatgaacaagtcatttccagaattaggaatcaagaagactgattgcaaagagattcttg |
| | gatcgacacaaccatcttctactctggggtcgtaaattataatactgcaaatttcaagaaagaaatattattggatcgatcagccgg |
| | taaaaagacagcatttagtattaaacttgattacgttaaaaaacttattccagaaaccgctatggttaaaattttagaaaaattgta |
| | cgaggaagaggtgggtgtcgggatgtacgttctttatccatatggtggtattatggatgaaatatccgagtctgcaatcccatttcc |
| | acatagagcaggaataatgtatgaactttggtacaccgcaacctgggaaaaacaagaggataatgaaaagcacattaattgggtcag |
| | atccgtttataactttactaccccatacgtatcacaaaacccacgtcttgcctatttaaactatagagacttagatcttggtaagac |
| | aaatccagagtctcctaataactatactcaagcacgtatctggggagagaagtattttggtaagaatataatagattagtcaaggtt |
| | aaaacaaaggctgaccctaacaatttctttagaaacgagcagtccatcccaccacttccaccaagacaccattga |

| Gene symbol | Gene Sequence (start to stop) |
|---|---|
| CBCAS (no | MNPQENFLKCFSEYIPNNPANPKFIYTQHDQLYMSVLNSTIQNLRFTSDTTPKPLVIVTPSNVS |
| signal peptide) | HIQASILCSKKVGLQIRTRSGGHDAEGLSYISQVPFAIVDLRNMHTVKVDIHSQTAWVEAGAT |
| (SEQ ID NO: | LGEVYYWINEMNENFSFPGGYCPTVGVGGHFSGGGYGALMRNYGLAADNIIDAHLVNVDG |
| 35) | KVLDRKSMGEDLFVVAIRGGGGENFGIIAACKIKLVVVPSKATIFSVKKNMEIHGLVKLFNKW |
| | QNIAYKYDKDLMLTTHFRTRNITDNHGKNKTTVHGYFSSIFLGGVDSLVDLMNKSFPELGIK |
| | KTDCKELSWIDTTIFYSGVVNYNTANFKKEILLDRSAGKKTAFSIKLDYVKKLIPETAMVKILE |
| | KLYEEEVGVGMYVLYPYGGIMDEISESAIPFPHRAGIMYELWYTATWEKQEDNEKHINWVR |
| | SVYNFTTPYVSQNPRLAYLNYRDLDLGKTNPESPNNYTQARIVVGEKYFGKNFNRLVKVKTK |
| | ADPNNFFRNEQSIPPLPPRHH* |

Plasmid Charts

| Plasmid | | Bacterial | Yeast | | | |
|---|---|---|---|---|---|---|
| name | Gene | Marker | Marker | Base Vector | Promoter | Terminator |
| pAA2333 | CvLEU2 | Kan | LEU2 | pCR-BluntII-TOPO | LEU2 | LEU2 |
| pAA3060 | LEU2 KOa | Kan | URA3 | pCR-BluntII-TOPO | NA | NA |
| pAA0244 | CvURA3 | Kan | URA3 | pCR-BluntII-TOPO | URA3 | URA3 |
| pAA2417 | LEU2 KOb | Kan | URA3 | pCR-BluntII-TOPO | NA | NA |
| pAA0873 | AuACO1(CvCO) | Kan | NA | pCR-BluntII-TOPO | NA | NA |
| pAA0956 | AuACO1-LSK(CvCO) | Kan | NA | pCR-BluntII-TOPO | NA | NA |
| pAA0964 | AuACO1-LSK(CvCO) | Amp | URA3 | pAA0335 | PEX11 | PEX11 |
| pVZ3970 | CsOAS(CvCO) | Kan | NA | pCR-BluntII-TOPO | NA | NA |
| pVZ4009 | CsOAS(CvCO) | Kan | URA3 | pAA1164 | HDE | POX4 |
| pVZ3968 | CsOAC(CvCO) | Kan | NA | pCR-BluntII-TOPO | NA | NA |
| pVZ4008 | CsOAC(CvCO) | Kan | URA3 | pAA1164 | HDE | POX4 |
| pAA1417 | ACS2d | Kan | NA | pCR-BluntII-TOPO | NA | NA |
| PAA1222 | NA | Amp | NA | pUC19 | NA | NA |
| pVZ4045 | split LEU2 | Amp | NA | pAA1222 | NA | NA |
| pVZ4285 | ACS2d | Amp | LEU2 | pVZ4045 | HDE | POX4 |
| pVZ4105 | CvERG20 | Amp | LEU2 | pVZ4045 | POX18 | POX18 |
| pRB0073 | CsPT1-noSS(CvCO) | Amp | LEU2 | pVZ4045 | HDE | POX4 |
| pRB0074 | CsPT1(CvCO) | Amp | LEU2 | pVZ4045 | HDE | POX4 |
| pRB0075 | CsCBDAS-noSS(CvCO) | Amp | LEU2 | pVZ4045 | HDE | POX4 |
| pRB0076 | CvERG20(F95W, N126W) | Amp | LEU2 | pVZ4105 | POX18 | POX18 |
| PRB0077 | CsPT4-noSS(CvCO) | Amp | LEU2 | pVZ4045 | HDE | POX4 |
| PRB0085 | NphB(G286S, Y288A)(CvCO) | Amp | LEU2 | pVZ4045 | HDE | POX4 |
| pVZ4348 | ACS2d^{Δpts} | Amp | LEU2 | pVZ4285 | HDE | POX4 |
| pVZ4277 | CsAAE1(CvCO) | Kan | NA | pCR-BluntII-TOPO | NA | NA |
| pVZ4282 | CsAAE1(CvCO) | Amp | LEU2 | pVZ4045 | HDE | POX4 |
| pAA3169 | CsPT1(CvCO) | Kan | NA | pCR-BluntII-TOPO | NA | NA |
| PAA3636 | CsPT1(CvCO) | Kan | URA3 | pAA1922 | GPD | POX4 |
| pAA3171 | CsCBDAS(CvCO) | Kan | NA | pCR-BluntII-TOPO | NA | NA |
| pAA3632 | CsCBDAS(CvCO) | Kan | URA3 | pAA1922 | GPD | POX4 |

1. A recombinant microorganism engineered to biosynthetically produce a cannabinoid acid from fatty acids, the recombinant microorganism comprising:
a. a first engineered biosynthetic pathway to produce hexanoyl-CoA via degradation of fatty acids, preferably monounsaturated fatty acids, which fatty acids are supplied exogenously and/or via de novo fatty acid biosynthesis; and
b. a second engineered biosynthetic pathway to produce a cannabinoid acid from hexanoyl-CoA.

2. A recombinant microorganism according to claim 1 that is a recombinant yeast, optionally a recombinant Candida species, optionally Candida viswanathii.

3. A recombinant microorganism according to claim 1 comprising:
a. a heterologous fatty acyl-CoA oxidase gene;
b. a heterologous fatty acyl-CoA synthetase gene;
c. a heterologous polyketide synthase (PKS) or tetraketide synthase (TKS) gene;
d. a heterologous olivetolic acid cyclase (OAC) gene; and
e. a heterologous aromatic prenyltransferase (GOT) gene.

4. A recombinant microorganism according to claim 1 comprising:
a. a heterologous fatty acyl-CoA oxidase gene;
b. a heterologous fatty acyl-CoA synthetase gene;
c. a heterologous polyketide synthase (PKS) or tetraketide synthase (TKS) gene;
d. a heterologous olivetolic acid cyclase (OAC) gene;
e. a heterologous aromatic prenyltransferase (GOT) gene; and
f. at least one additional heterologous gene selected from the group consisting of a heterologous cannabidiolic acid synthase (CBDAS) gene, a heterologous cannabichromenic acid synthase (CBCAS) gene, a heterologous cannabinoid acid synthase gene, and a heterologous tetrahydrocannabinolic acid synthase (THCAS) gene.

5. A recombinant microorganism comprising an expression cassette that directs expression of a fatty acyl-CoA oxidase enzyme encoded by a heterologous fatty acyl-CoA oxidase gene, which fatty acyl-CoA oxidase enzyme catalyzes production of hexanoate.

6. A recombinant microorganism according to claim 5 wherein the fatty acyl-CoA oxidase enzyme has an amino acid sequence having an identity of at least about 50% to 100% of the amino acid sequence of SEQ ID NO: 2.

7. A biosynthetic production method for a cannabinoid acid, comprising cultivating a recombinant microorganism according to claim 1 in a feedstock comprising a carbon source, optionally glycerol or a fatty acid (or mixture of fatty acid species), under growth conditions that promote production of the cannabinoid acid.

8. A biosynthetic production method for a cannabinoid, comprising cultivating a recombinant microorganism according to claim 1 in a feedstock comprising a carbon source, optionally glycerol or a fatty acid (or mixture of fatty acid species), under growth conditions that promote production of a cannabinoid acid that can be converted to the cannabinoid, followed by converting the cannabinoid acid to the cannabinoid by decarboxylation, optionally non-enzymatic decarboxylation, optionally heat, and optionally then recovering the cannabinoid so produced.

9. A biosynthetic production method for cannabigerolic acid, comprising cultivating a recombinant microorganism according to claim 1 in a feedstock comprising a carbon source, optionally glycerol or a fatty acid (or mixture of fatty acid species), under growth conditions that promote production of cannabigerolic acid.

10. A biosynthetic production method for cannabigerol, comprising cultivating a recombinant microorganism according to claim 1 in a feedstock comprising a carbon source, optionally glycerol or a fatty acid (or mixture of fatty acid species), under growth conditions that promote production of cannabigerolic acid, followed by converting the cannabigerolic acid to cannabigerol by decarboxylation, optionally non-enzymatic decarboxylation, optionally heat, and optionally then recovering the cannabigerol so produced.

11. A biosynthetic production method for cannabidiolic acid, comprising cultivating a recombinant microorganism according to claim 1 in a feedstock comprising a carbon source, optionally glycerol or a fatty acid (or mixture of fatty acid species), under growth conditions that promote production of cannabidiolic acid.

12. A biosynthetic production method for cannabidiol, comprising cultivating a recombinant microorganism according to claim 1 in a feedstock comprising a carbon source, optionally glycerol or a fatty acid (or mixture of fatty acid species), under growth conditions that promote production of cannabidiolic acid, followed by converting the cannabidiolic acid to cannabidiol by decarboxylation, optionally non-enzymatic decarboxylation, optionally heat, and optionally then recovering the cannabidiol so produced.

13. A biosynthetic production method for cannabichromenic acid, comprising cultivating a recombinant microorganism according to claim 1 in a feedstock comprising a carbon source, optionally glycerol or a fatty acid (or mixture of fatty acid species), under growth conditions that promote production of cannabichromenic acid.

14. A biosynthetic production method for cannabichromene, comprising cultivating a recombinant microorganism according to claim 1 in a feedstock comprising a carbon source, optionally glycerol or a fatty acid (or mixture of fatty acid species), under growth conditions that promote production of cannabichromenic acid, followed by converting the cannabichromenic acid to cannabichromene by decarboxylation, optionally non-enzymatic decarboxylation, optionally heat, and optionally then recovering the cannabichromene so produced.

15. A biosynthetic production method for tetrahydrocannabinolic acid, comprising cultivating a recombinant microorganism according to claim 1 in a feedstock comprising a carbon source, optionally glycerol or a fatty acid (or mixture of fatty acid species), under growth conditions that promote production of tetrahydrocannabinolic acid.

16. A biosynthetic production method for tetrahydrocannabinol, comprising cultivating a recombinant microorganism according to claim 1 in a feedstock comprising a carbon source, optionally glycerol or a fatty acid (or mixture of fatty acid species), under growth conditions that promote production of tetrahydrocannabinolic acid, followed by converting the tetrahydrocannabinolic acid to tetrahydrocannabinol by decarboxylation, optionally non-enzymatic decarboxylation, optionally heat, and optionally then recovering the tetrahydrocannabinol so produced.

17. A method according to claim 7 wherein the yield of the cannabinoid acid or cannabinoid is about 0.001 g/L to about 100 g/L.

18. A method according to claim 7 wherein the feedstock comprises one or more fatty acid species.

19. A method according to claim 7 wherein the cannabinoid acid or cannabinoid is recovered from growth medium.
